(19) United States
(12) Patent Application Publication
(10) Pub. No.: US 2023/0389422 A1
(43) Pub. Date: Nov. 30, 2023

(54) ORGANIC ELECTROLUMINESCENT COMPOUND AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Chi-Sik KIM, Gyeonggi-do (KR); Hyo-Soon PARK, Gyeonggi-do (KR); Su-Hyun LEE, Gyeonggi-do (KR); Kyoung-Jin PARK, Gyeonggi-do (KR)

(21) Appl. No.: 18/364,653

(22) Filed: Aug. 3, 2023

Related U.S. Application Data
(63) Continuation of application No. 17/000,977, filed on Aug. 24, 2020.

(30) Foreign Application Priority Data
Sep. 5, 2019 (KR) 10-2019-0109845

(51) Int. Cl. H10K 85/60; C07D 405/14; C07D 409/14; C09K 11/06; H10K 50/12

(52) U.S. Cl. CPC H10K 85/6576; C07D 405/14; C07D 409/14; C09K 11/06; H10K 50/12; H10K 85/654; H10K 85/6572; H10K 85/6574; C09K 2211/1029; C09K 2211/1059; C09K 2211/1088; H10K 2101/90

(57) ABSTRACT

The present disclosure relates to an organic electroluminescent compound represented by formula 1 and an organic electroluminescent device comprising the same. By comprising the organic electroluminescent compound according to the present disclosure, it is possible to provide an organic electroluminescent device with a longer lifetime property compared to conventional organic electroluminescent devices.

### CLAIM OF BENEFIT OF PRIOR APPLICATION

This application claims priority under 35 U.S.C. § 120 from U.S. patent application Ser. No. 17/000,977 filed Aug. 24, 2020, which is the Convention Filing of KR10-2019-0109845 filed Sep. 05, 2019, all of which are incorporated by reference herein in their entirety.

### TECHNICAL FIELD

The present disclosure relates to an organic electroluminescent compound and an organic electroluminescent device comprising the same.

### BACKGROUND ART

An electroluminescent (EL) device is a self-light-emitting display device which has advantages in that it provides a wider viewing angle, a greater contrast ratio, and a faster response time. The first organic electroluminescent device was developed by Eastman Kodak in 1987, by using small aromatic diamine molecules and aluminum complexes as materials for forming a light-emitting layer (see Appl. Phys. Lett. 51, 913, 1987).

An organic electroluminescent device (OLED) changes electric energy into light by applying electricity to an organic electroluminescent material, and commonly comprises an anode, a cathode, and an organic layer formed between the two electrodes. The organic layer of the OLED may comprise a hole injection layer, a hole transport layer, a hole auxiliary layer, a light-emitting auxiliary layer, an electron blocking layer, a light-emitting layer, an electron buffer layer, a hole blocking layer, an electron transport layer, an electron injection layer, etc. The materials used in the organic layer can be classified into a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc., depending on their functions. In the OLED, holes from the anode and electrons from the cathode are injected into a light-emitting layer by the application of electric voltage, and excitons having high energy are produced by the recombination of the holes and electrons. The organic light-emitting compound moves into an excited state by the energy and emits light from energy when the organic light-emitting compound returns to the ground state from the excited state.

At present, OLEDs primarily use phosphorescent materials having excellent luminous efficiency in panel implementation. In many applications such as TVs and lightings, the lifetime of OLED is insufficient. Therefore, a new light-emitting material having properties of a longer lifetime has been required for using a display for a longer period of time. For example, Korean Patent No. 10-1396171 discloses a group of organic electroluminescent compounds. However, said reference does not specifically disclose an organic electroluminescent compound claimed in the present disclosure. In addition, development of a light-emitting material having improved performance compared to the organic electroluminescent compounds disclosed in said reference is required, for example, an improved lifetime property, alone or in combination with another host material.

### DISCLOSURE OF INVENTION

### Technical Problem

The objective of the present disclosure is to provide an organic electroluminescent compound effective for producing an organic electroluminescent device with a longer lifetime property compared to conventional organic electroluminescent devices. Solution to Problem

The present inventors found that the above objective can be achieved by an organic electroluminescent compound represented by the following formula 1:

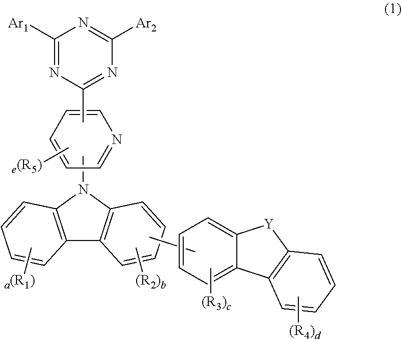

wherein

Ar₁ and Ar₂, each independently, represent a substituted or unsubstituted (C6-C30)aryl, or a substituted or unsubstituted (3- to 30-membered)heteroaryl;

Y represents O or S;

R₁ to R₅, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted (C1-C30)alkyl(C6-C30)arylamino, or a substituted or unsubstituted mono- or di- (C6-C30)arylamino; or adjacent ones of R₁ to R₅ may be linked to each other to form a ring(s); and

a and d, each independently, represent an integer of 1 to 4; b, c, and e, each independently, represent an integer of 1 to 3; where each of a to e is an integer of 2 or more, each of R₁, each of R₂, each of R₃, each of R₄, and each of R₅ may be the same or different.

### Advantageous Effects of Invention

An organic electroluminescent device comprising the organic electroluminescent compound according to the present disclosure can achieve a longer lifetime property compared to conventional organic electroluminescent devices.

### Mode for the Invention

Hereinafter, the present disclosure will be described in detail. However, the following description is intended to explain the present disclosure, and is not meant in any way to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material, an electron buffer material, a hole blocking material, an electron transport material, or an electron injection material, etc.

The term “a plurality of host materials” in the present disclosure means a host material comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of host materials of the present disclosure may be a combination of at least two host materials, and selectively may further comprise conventional materials comprised in an organic electroluminescent material. At least two compounds comprised in the plurality of host materials may be comprised together in one light-emitting layer or may respectively be comprised in different light-emitting layers. For example, the at least two host materials may be mixture-evaporated or co-evaporated, or may be individually evaporated.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The alkyl may include methyl, ethyl, n-propyl, iso-propyl, n-butyl, iso-butyl, tert-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The alkenyl may include vinyl, 1-propenyl, 2-propenyl, 1-butenyl, 2-butenyl, 3-butenyl, 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The cycloalkyl may include cyclopropyl, cyclobutyl, cyclopentyl, cyclohexyl, etc. The term “(3- to 7-membered)heterocycloalkyl” is meant to be a cycloalkyl having 3 to 7 ring backbone atoms, preferably 5 to 7 ring backbone atoms, and including at least one heteroatom selected from the group consisting of B, N, O, S, Si, and P, and preferably the group consisting of O, S, and N. The heterocycloalkyl may include tetrahydrofuran, pyrrolidine, thiolan, tetrahydropyran, etc. The term “(C6-C30)aryl” is meant to be a monocyclic or fused ring radical derived from an aromatic hydrocarbon having 6 to 30 ring backbone carbon atoms, in which the number of the ring backbone carbon atoms is preferably 6 to 25, more preferably 6 to 18. The aryl may be partially saturated, and may comprise a spiro structure. The aryl may include phenyl, biphenyl, terphenyl, naphthyl, binaphthyl, phenylnaphthyl, naphthylphenyl, phenylterphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, azulenyl, etc. More specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzofluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenylyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, etc.

The term “(3- to 30-membered)heteroaryl” is an aryl having 3 to 30 ring backbone atoms and including at least one, preferably 1 to 4 heteroatoms selected from the group consisting of B, N, O, S, Si, and P. The heteroaryl may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, etc. More specifically, the heteroaryl may include 1-pyrrolyl, 2-pyrrolyl, 3-pyrrolyl, pyrazinyl, 2-pyridinyl, 2-pyrimidinyl, 4-pyrimidinyl, 5-pyrimidinyl, 6-pyrimidinyl, 1,2,3-triazin-4-yl, 1 ,2,4-triazin-3-yl, 1 ,3,5-triazin-2-yl, 1 -imidazolyl, 2-imidazolyl, 1 -pyrazolyl, 1 -indolidinyl, 2-indolidinyl, 3-indolidinyl, 5-indolidinyl, 6-indolidinyl, 7-indolidinyl, 8-indolidinyl, 2-imidazopyridinyl, 3-imidazopyridinyl, 5-imidazopyridinyl, 6-imidazopyridinyl, 7-imidazopyridinyl, 8-imidazopyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyI-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tent-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, etc. “Halogen” includes F, Cl, Br, and I.

In addition, “ortho (o-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, “substituted” in the expression “substituted or unsubstituted” means that a hydrogen atom in a certain functional group is replaced with another atom or another functional group, i.e., a substituent. In the present disclosure, the substituents of the substituted alkyl, the substituted aryl, the substituted heteroaryl, the substituted trialkylsilyl, the substituted dialkylarylsilyl, the substituted alkyldiarylsilyl, the substituted triarylsilyl, the substituted mono- or di- alkylamino, the substituted alkylarylamino, and the substituted mono- or di- arylamino, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di- (C1-C30)alkylamino; a mono- or di- (C6-C30)arylamino unsubstituted or substituted with a (C1-C30)alkyl(s); a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituents, each independently, are at least one selected from the group consisting of deuterium; a (C1-C20)alkyl; a (5- to 25-membered)heteroaryl unsubstituted or substituted with a (C6-C25)aryl(s); an unsubstituted (C6-C25)aryl; and a tri(C6-C25)arylsilyl. According to another embodiment of the present disclosure, the substituents, each independently, are at least one selected from the group consisting of deuterium; a (C1-C10)alkyl; a (5- to 20-membered)heteroaryl unsubstituted or substituted with a (C6-C18)aryl(s); an unsubstituted (C6-C18)aryl; and a tri(C6-C18)arylsilyl. For example, the substituents, each independently, may be at least one selected from the group consisting of a methyl, a tert-butyl, a phenyl, a naphthyl, a pyridyl unsubstituted or substituted with a phenyl(s), and a triphenylsilyl.

Herein, a ring formed by a linkage of adjacent substituents means that at least two adjacent substituents are linked to or fused with each other to form a substituted or unsubstituted mono- or polycyclic (3- to 30-membered) alicyclic or aromatic ring, or the combination thereof; preferably, a substituted or unsubstituted mono- or polycyclic (3- to 26-membered) alicyclic or aromatic ring, or the combination thereof; and more preferably, an unsubstituted mono- or polycyclic (5- to 10-membered) aromatic ring. For example, the ring may be a benzene ring, an indene ring, an indole ring, a benzofuran ring, or a benzothiophene ring, etc. In addition, the ring may contain at least one heteroatom selected from B, N, O, S, Si, and P, preferably at least one heteroatom selected from N, O, and S.

In the formulas of the present disclosure, the heteroaryl and the heterocycloalkyl, each independently, may contain at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono-or di- (C6-C30)arylamino, and a substituted or unsubstituted (C1-C30)alkyl(C6-C30)arylamino.

Hereinafter, the compound represented by formula 1 will be described in more detail.

In formula 1, Ar₁ and Ar₂, each independently, represent a substituted or unsubstituted (C6-C30)aryl, or a substituted or unsubstituted (3- to 30-membered)heteroaryl. Ar₁ and Ar₂ may be the same or different. According to one embodiment of the present disclosure, Ar₁ and Ar₂, each independently, represent a substituted or unsubstituted (C6-C25)aryl. According to another embodiment of the present disclosure, Ar₁ and Ar₂, each independently, represent a (C6-C18)aryl unsubstituted or substituted with a (C1-C6)alkyl(s). According to a further embodiment of the present disclosure, Ar₁ and Ar₂, each independently, represent a (C6-C12)aryl unsubstituted or substituted with a (C1-C6)alkyl(s). For example, Ar₁ and Ar₂, each independently, may be a phenyl unsubstituted or substituted with a tert-butyl(s), or a biphenyl.

In formula 1, Y represents O or S.

In formula 1, R₁ to R₅, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted (C1-C30)alkyl(C6-C30)arylamino, or a substituted or unsubstituted mono- or di- (C6-C30)arylamino; or adjacent ones of R₁ to R₅ may be linked to each other to form a ring(s). At least two adjacent R₁'s may be linked to each other to form a ring(s), at least two adjacent R₂'s may be linked to each other to form a ring(s), at least two adjacent R₃'s may be linked to each other to form a ring(s), at least two adjacent R₄'s may be linked to each other to form a ring(s), and/or at least two adjacent R₅'s may be linked to each other to form a ring(s). For example, R₁ to R₅ may be hydrogen.

In formula 1, a and d, each independently, represent an integer of 1 to 4; b, c, and e, each independently, represent an integer of 1 to 3; where each of a to e is an integer of 2 or more, each of R₁, each of R₂, each of R₃, each of R₄, and each of R₅ may be the same or different. For example, each of a to d may be an integer of 1.

The formula 1 may be represented by any one of the following formulas 1-1 to 1-3.

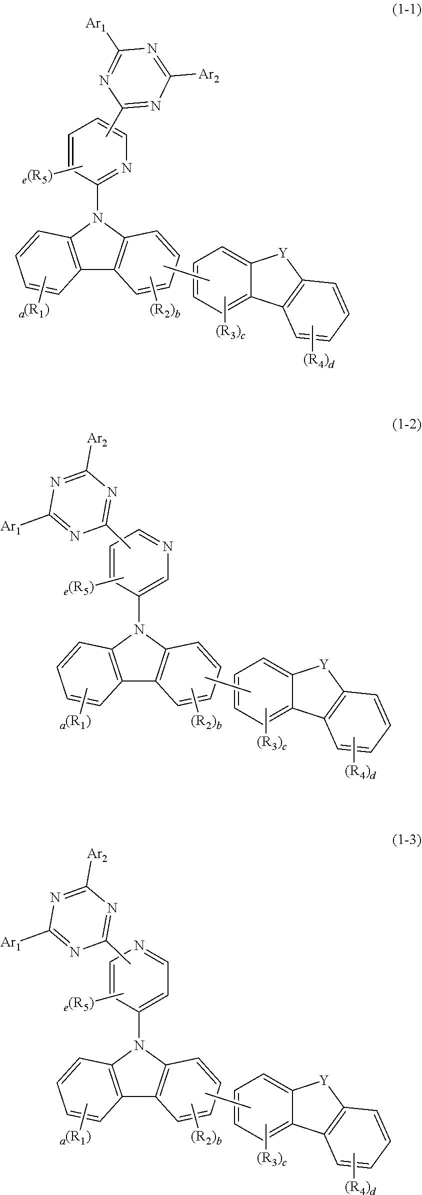

In formulas 1-1 to 1-3, Ar₁, Ar₂, Y, R₁ to R₅, and a to d are as defined in formula 1.

In formula 1,

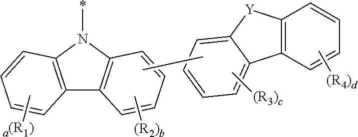

may be represented by any one of the following formulas 1-4 to 1-6, in which * represents a bonding site to pyridylene in formula 1.

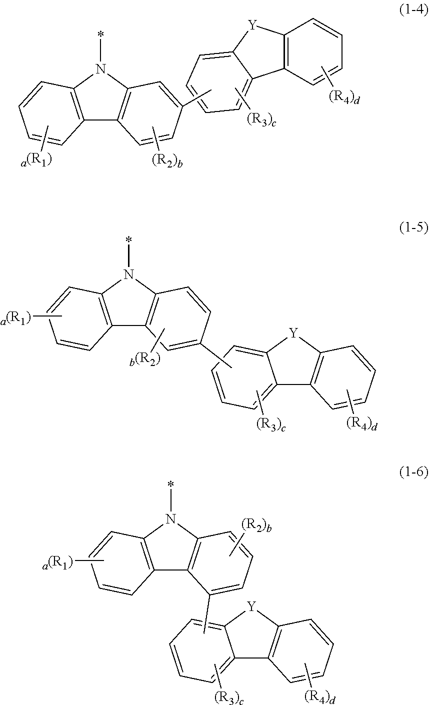

In formulas 1-4 to 1-6, Y, R₁ to R₄, and a to d are as defined in formula 1.

The compound represented by formula 1 may be selected from the group consisting of the following compounds, but is not limited thereto.

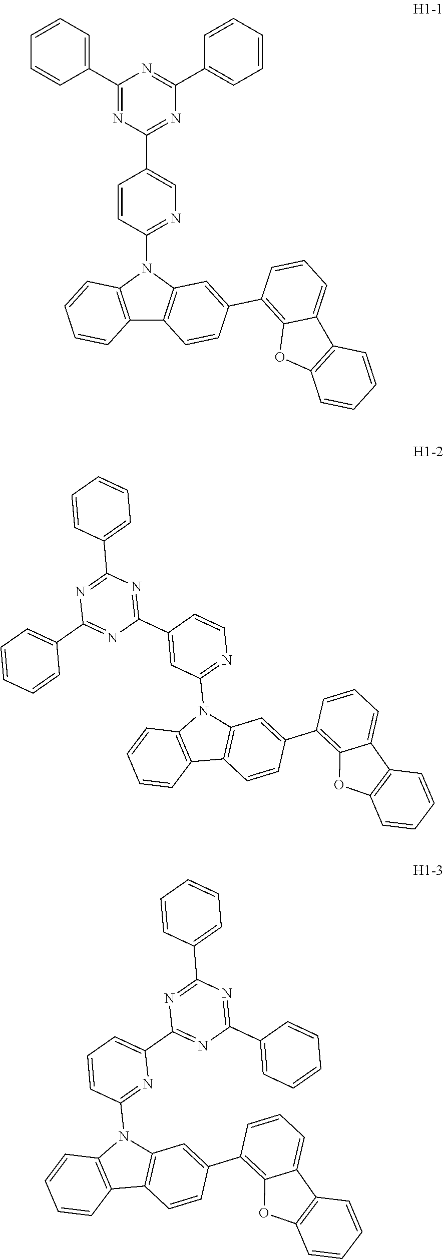 [SMILES: c1ccc(-c2nc(-c3ccccc3)nc(-c3ccc(-n4c5ccccc5c5ccc(-c6cccc7c6oc6ccccc67)cc54)nc3)n2)cc1 ; c1ccc(-c2nc(-c3ccccc3)nc(-c3ccnc(-n4c5ccccc5c5ccc(-c6cccc7c6oc6ccccc67)cc54)c3)n2)cc1 ; c1ccc(-c2nc(-c3ccccc3)nc(-c3cccc(-n4c5ccccc5c5ccc(-c6cccc7c6oc6ccccc67)cc54)n3)n2)cc1]

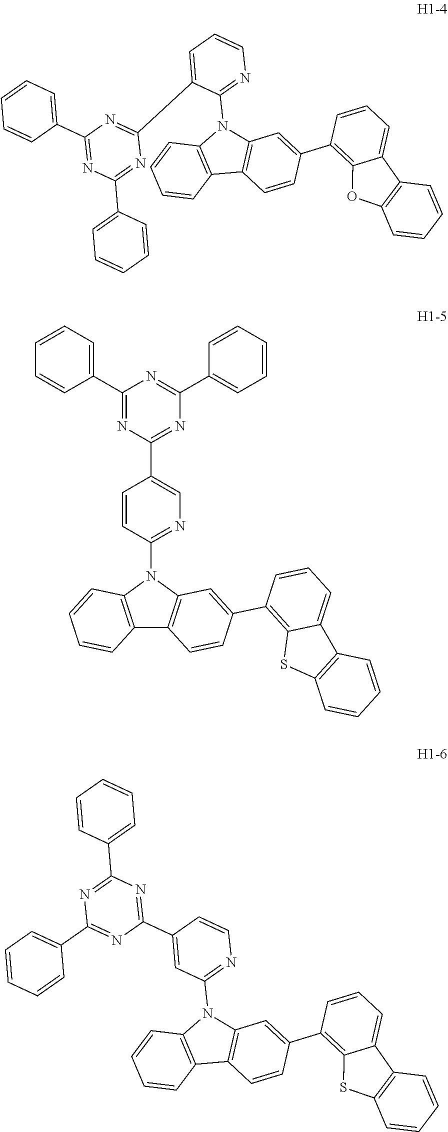 [SMILES: c1ccc(-c2nc(-c3ccccc3)nc(-c3cccnc3-n3c4ccccc4c4ccc(-c5cccc6c5oc5ccccc56)cc43)n2)cc1 ; c1ccc(-c2nc(-c3ccccc3)nc(-c3ccc(-n4c5ccccc5c5ccc(-c6cccc7c6sc6ccccc67)cc54)nc3)n2)cc1 ; c1ccc(-c2nc(-c3ccccc3)nc(-c3ccnc(-n4c5ccccc5c5ccc(-c6cccc7c6sc6ccccc67)cc54)c3)n2)cc1]

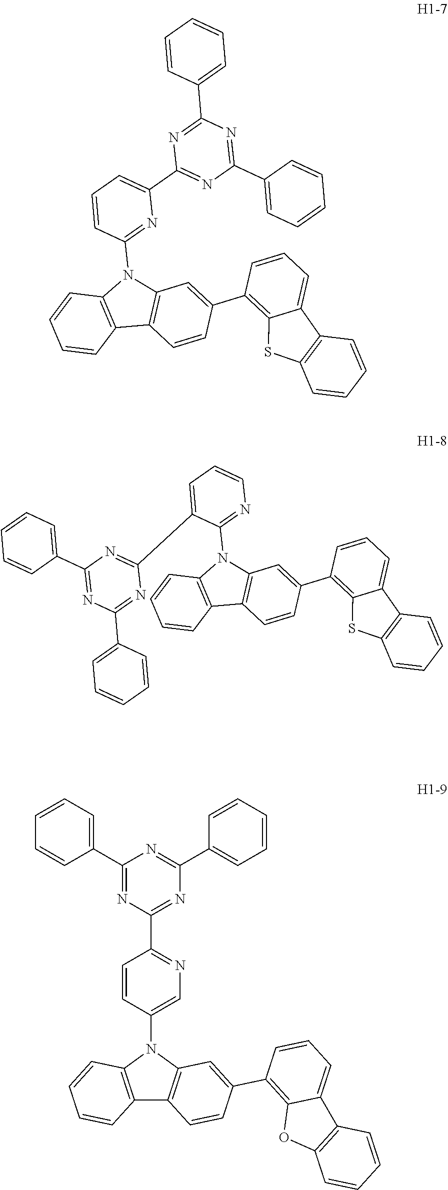 [SMILES: c1ccc(-c2nc(-c3ccccc3)nc(-c3cccc(-n4c5ccccc5c5ccc(-c6cccc7c6sc6ccccc67)cc54)n3)n2)cc1 ; c1ccc(-c2nc(-c3ccccc3)nc(-c3cccnc3-n3c4ccccc4c4ccc(-c5cccc6c5sc5ccccc56)cc43)n2)cc1 ; c1ccc(-c2nc(-c3ccccc3)nc(-c3ccc(-n4c5ccccc5c5ccc(-c6cccc7c6oc6ccccc67)cc54)cn3)n2)cc1]

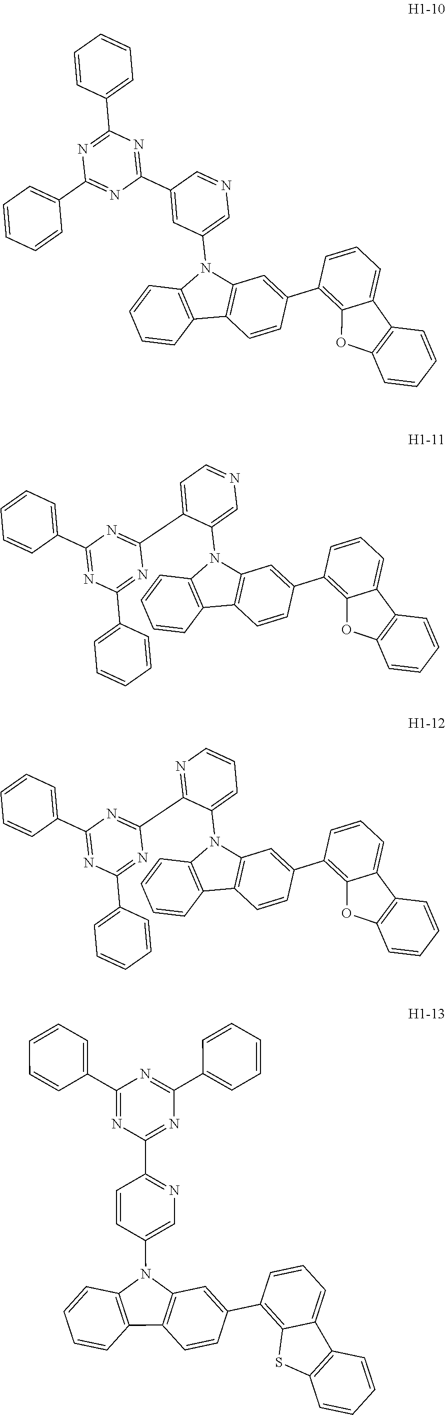 [SMILES: c1ccc(-c2nc(-c3ccccc3)nc(-c3cncc(-n4c5ccccc5c5ccc(-c6cccc7c6oc6ccccc67)cc54)c3)n2)cc1 ; c1ccc(-c2nc(-c3ccccc3)nc(-c3ccncc3-n3c4ccccc4c4ccc(-c5cccc6c5oc5ccccc56)cc43)n2)cc1 ; c1ccc(-c2nc(-c3ccccc3)nc(-c3ncccc3-n3c4ccccc4c4ccc(-c5cccc6c5oc5ccccc56)cc43)n2)cc1 ; c1ccc(-c2nc(-c3ccccc3)nc(-c3ccc(-n4c5ccccc5c5ccc(-c6cccc7c6sc6ccccc67)cc54)cn3)n2)cc1]

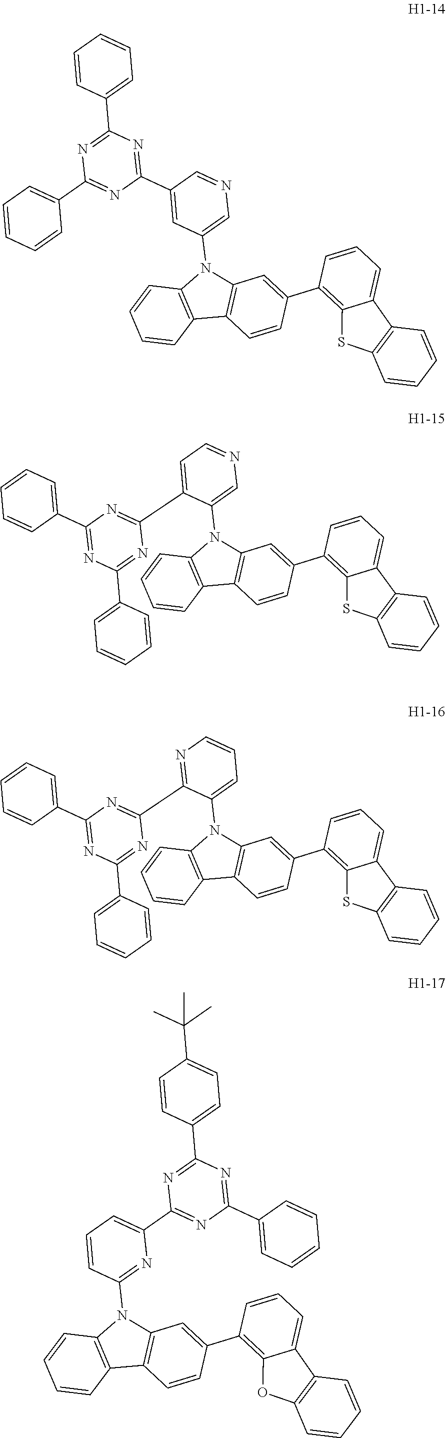 [SMILES: c1ccc(-c2nc(-c3ccccc3)nc(-c3cncc(-n4c5ccccc5c5ccc(-c6cccc7c6sc6ccccc67)cc54)c3)n2)cc1 ; c1ccc(-c2nc(-c3ccccc3)nc(-c3ccncc3-n3c4ccccc4c4ccc(-c5cccc6c5sc5ccccc56)cc43)n2)cc1 ; c1ccc(-c2nc(-c3ccccc3)nc(-c3ncccc3-n3c4ccccc4c4ccc(-c5cccc6c5sc5ccccc56)cc43)n2)cc1 ; CC(C)(C)c1ccc(-c2nc(-c3ccccc3)nc(-c3cccc(-n4c5ccccc5c5ccc(-c6cccc7c6oc6ccccc67)cc54)n3)n2)cc1]

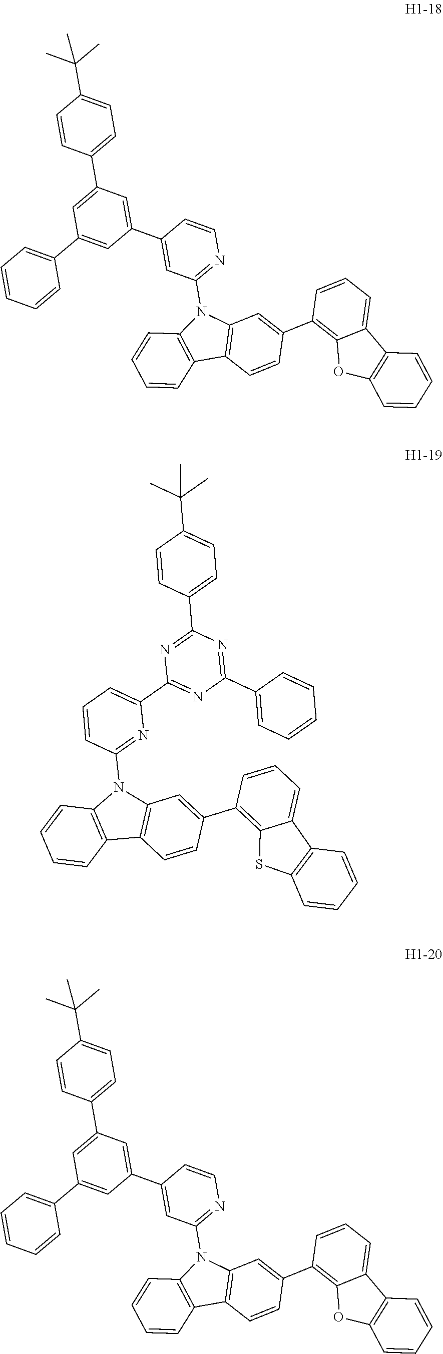 [SMILES: CC(C)(C)c1ccc(-c2cc(-c3ccccc3)cc(-c3ccnc(-n4c5ccccc5c5ccc(-c6cccc7c6oc6ccccc67)cc54)c3)c2)cc1 ; CC(C)(C)c1ccc(-c2nc(-c3ccccc3)nc(-c3cccc(-n4c5ccccc5c5ccc(-c6cccc7c6sc6ccccc67)cc54)n3)n2)cc1 ; CC(C)(C)c1ccc(-c2cc(-c3ccccc3)cc(-c3ccnc(-n4c5ccccc5c5ccc(-c6cccc7c6oc6ccccc67)cc54)c3)c2)cc1]

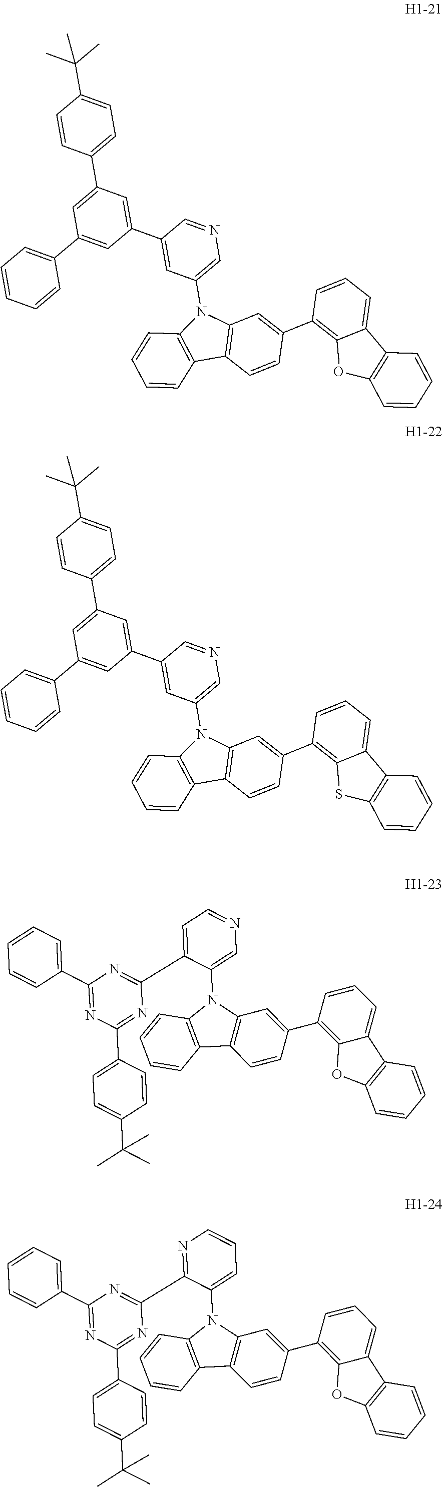 [SMILES: CC(C)(C)c1ccc(-c2cc(-c3ccccc3)cc(-c3cncc(-n4c5ccccc5c5ccc(-c6cccc7c6oc6ccccc67)cc54)c3)c2)cc1 ; CC(C)(C)c1ccc(-c2cc(-c3ccccc3)cc(-c3cncc(-n4c5ccccc5c5ccc(-c6cccc7c6sc6ccccc67)cc54)c3)c2)cc1 ; CC(C)(C)c1ccc(-c2nc(-c3ccccc3)nc(-c3ccncc3-n3c4ccccc4c4ccc(-c5cccc6c5oc5ccccc56)cc43)n2)cc1 ; CC(C)(C)c1ccc(-c2nc(-c3ccccc3)nc(-c3ncccc3-n3c4ccccc4c4ccc(-c5cccc6c5oc5ccccc56)cc43)n2)cc1]

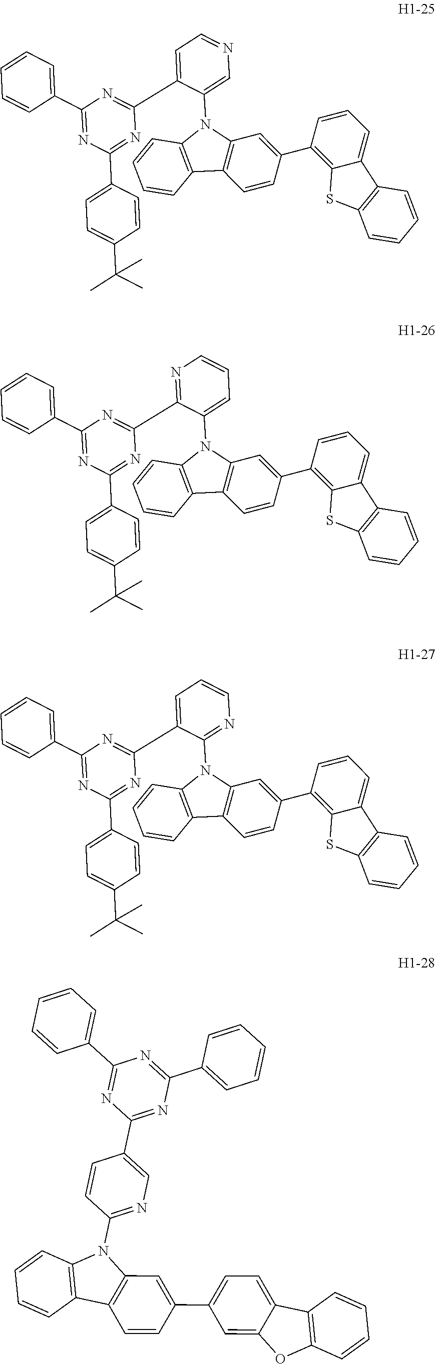 [SMILES: CC(C)(C)c1ccc(-c2nc(-c3ccccc3)nc(-c3ccncc3-n3c4ccccc4c4ccc(-c5cccc6c5sc5ccccc56)cc43)n2)cc1 ; CC(C)(C)c1ccc(-c2nc(-c3ccccc3)nc(-c3ncccc3-n3c4ccccc4c4ccc(-c5cccc6c5sc5ccccc56)cc43)n2)cc1 ; CC(C)(C)c1ccc(-c2nc(-c3ccccc3)nc(-c3cccnc3-n3c4ccccc4c4ccc(-c5cccc6c5sc5ccccc56)cc43)n2)cc1 ; c1ccc(-c2nc(-c3ccccc3)nc(-c3ccc(-n4c5ccccc5c5ccc(-c6ccc7c(c6)oc6ccccc67)cc54)nc3)n2)cc1]

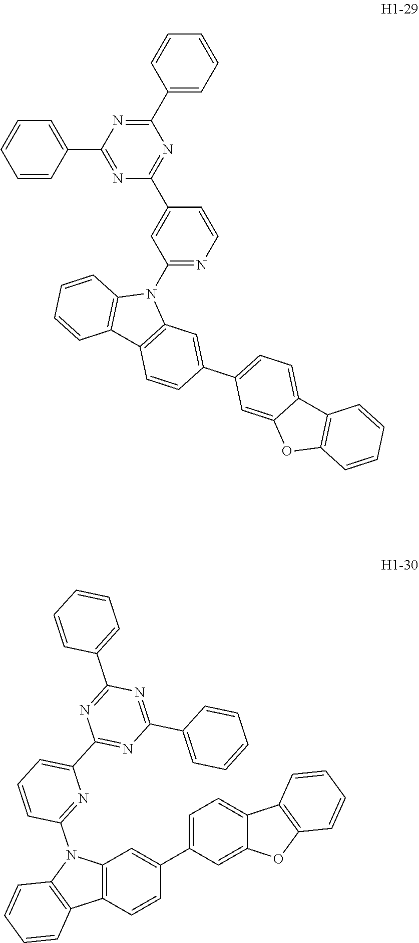 [SMILES: c1ccc(-c2nc(-c3ccccc3)nc(-c3ccnc(-n4c5ccccc5c5ccc(-c6ccc7c(c6)oc6ccccc67)cc54)c3)n2)cc1 ; c1ccc(-c2nc(-c3ccccc3)nc(-c3cccc(-n4c5ccccc5c5ccc(-c6ccc7c(c6)oc6ccccc67)cc54)n3)n2)cc1]

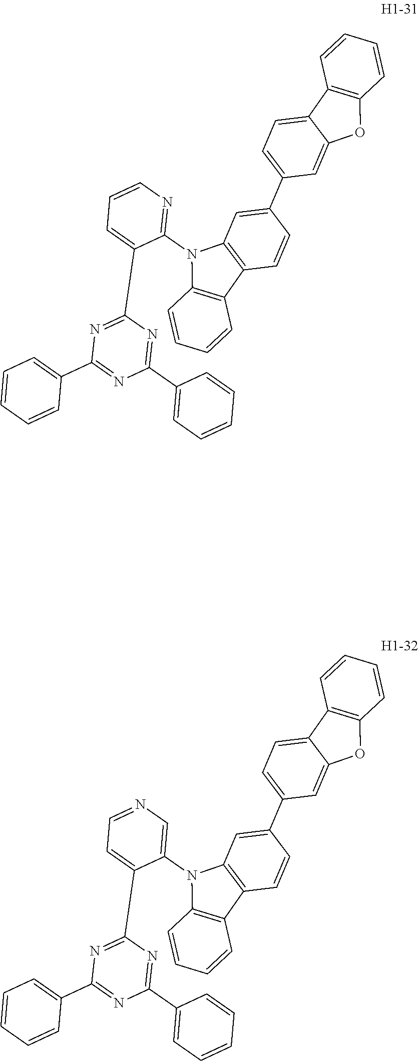 [SMILES: c1ccc(-c2nc(-c3ccccc3)nc(-c3cccnc3-n3c4ccccc4c4ccc(-c5ccc6c(c5)oc5ccccc56)cc43)n2)cc1 ; c1ccc(-c2nc(-c3ccccc3)nc(-c3ccncc3-n3c4ccccc4c4ccc(-c5ccc6c(c5)oc5ccccc56)cc43)n2)cc1]

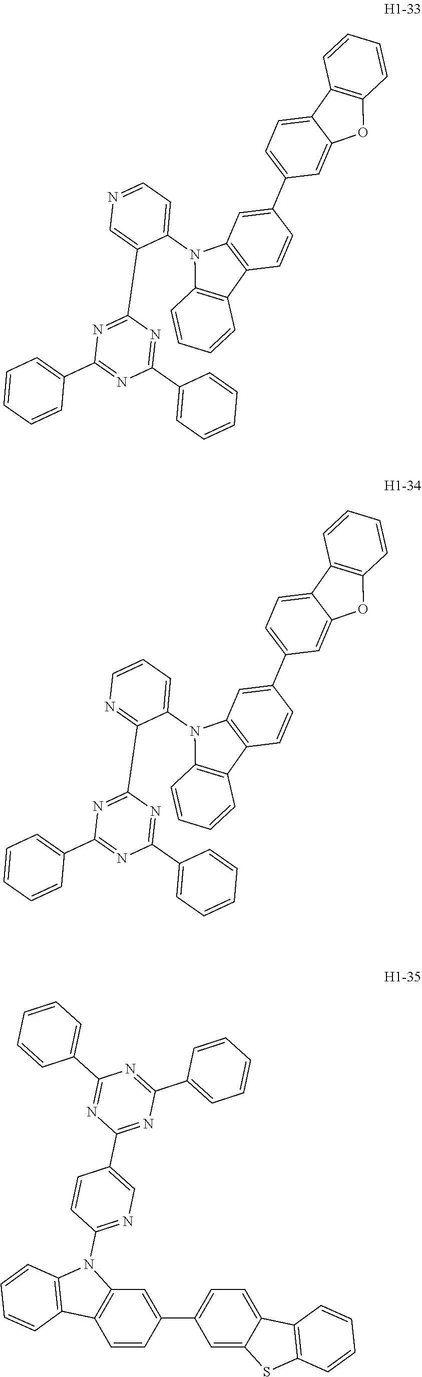 [SMILES: c1ccc(-c2nc(-c3ccccc3)nc(-c3cnccc3-n3c4ccccc4c4ccc(-c5ccc6c(c5)oc5ccccc56)cc43)n2)cc1 ; c1ccc(-c2nc(-c3ccccc3)nc(-c3ncccc3-n3c4ccccc4c4ccc(-c5ccc6c(c5)oc5ccccc56)cc43)n2)cc1 ; c1ccc(-c2nc(-c3ccccc3)nc(-c3ccc(-n4c5ccccc5c5ccc(-c6ccc7c(c6)sc6ccccc67)cc54)nc3)n2)cc1]

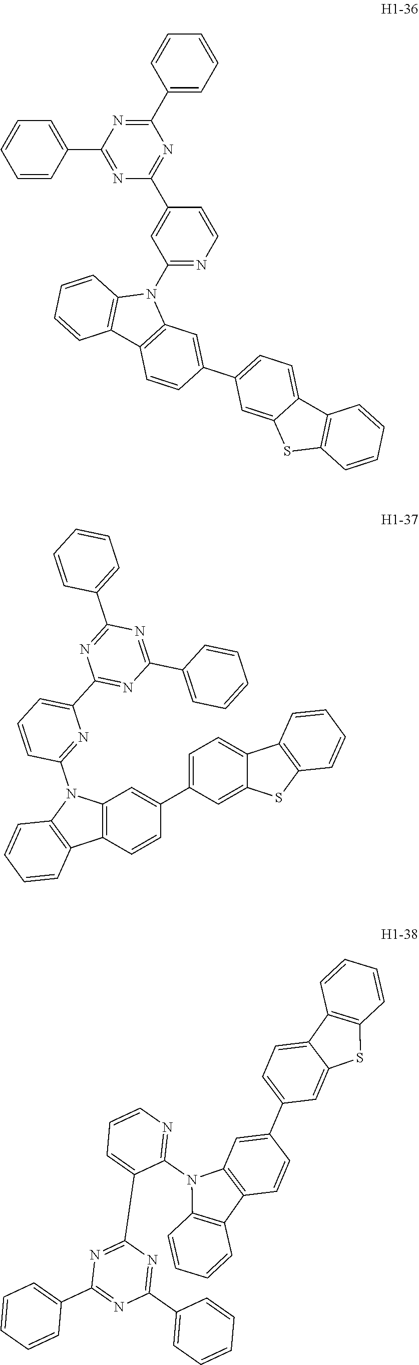 [SMILES: c1ccc(-c2nc(-c3ccccc3)nc(-c3ccnc(-n4c5ccccc5c5ccc(-c6ccc7c(c6)sc6ccccc67)cc54)c3)n2)cc1 ; c1ccc(-c2nc(-c3ccccc3)nc(-c3cccc(-n4c5ccccc5c5ccc(-c6ccc7c(c6)sc6ccccc67)cc54)n3)n2)cc1 ; c1ccc(-c2nc(-c3ccccc3)nc(-c3cccnc3-n3c4ccccc4c4ccc(-c5ccc6c(c5)sc5ccccc56)cc43)n2)cc1]

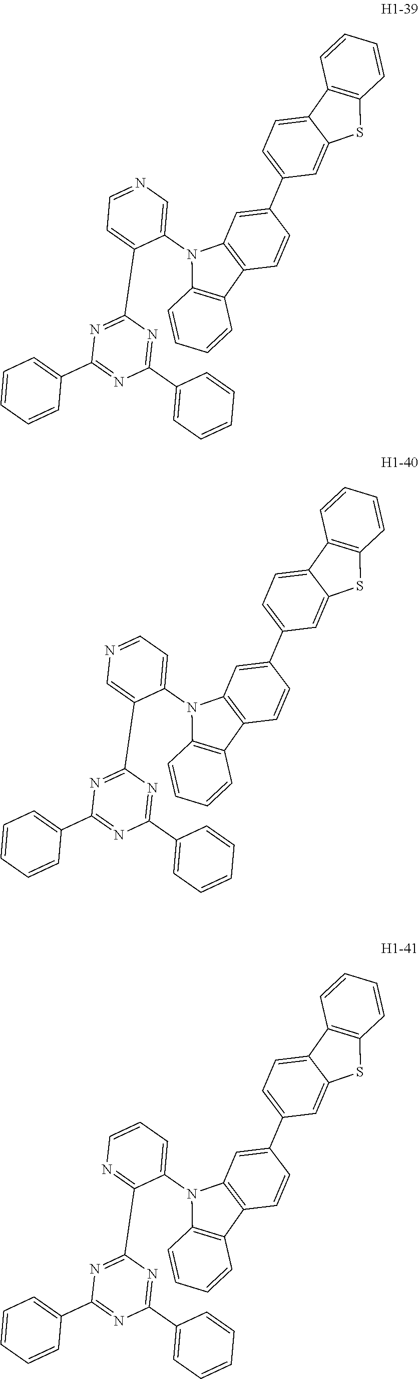 [SMILES: c1ccc(-c2nc(-c3ccccc3)nc(-c3ccncc3-n3c4ccccc4c4ccc(-c5ccc6c(c5)sc5ccccc56)cc43)n2)cc1 ; c1ccc(-c2nc(-c3ccccc3)nc(-c3cnccc3-n3c4ccccc4c4ccc(-c5ccc6c(c5)sc5ccccc56)cc43)n2)cc1 ; c1ccc(-c2nc(-c3ccccc3)nc(-c3ncccc3-n3c4ccccc4c4ccc(-c5ccc6c(c5)sc5ccccc56)cc43)n2)cc1]

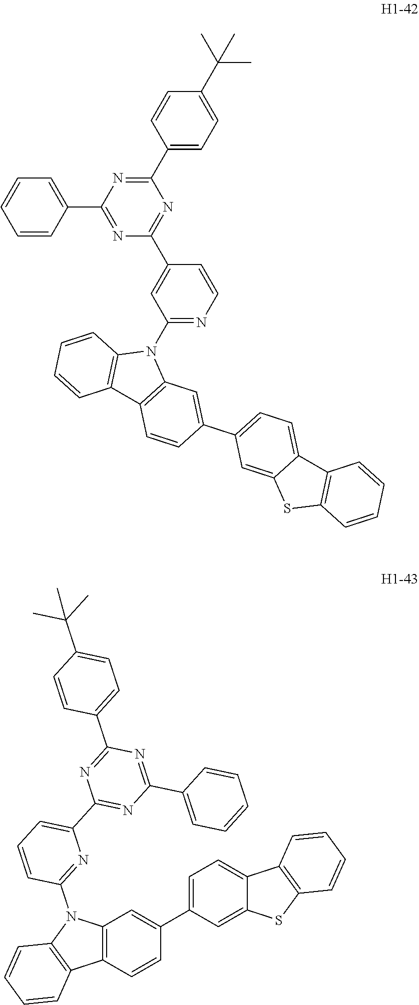 [SMILES: CC(C)(C)c1ccc(-c2nc(-c3ccccc3)nc(-c3ccnc(-n4c5ccccc5c5ccc(-c6ccc7c(c6)sc6ccccc67)cc54)c3)n2)cc1 ; CC(C)(C)c1ccc(-c2nc(-c3ccccc3)nc(-c3cccc(-n4c5ccccc5c5ccc(-c6ccc7c(c6)sc6ccccc67)cc54)n3)n2)cc1]

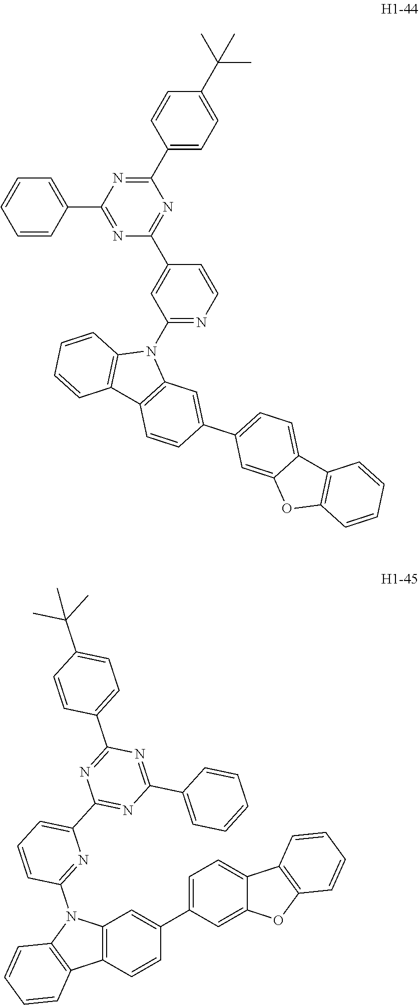 [SMILES: CC(C)(C)c1ccc(-c2nc(-c3ccccc3)nc(-c3ccnc(-n4c5ccccc5c5ccc(-c6ccc7c(c6)oc6ccccc67)cc54)c3)n2)cc1 ; CC(C)(C)c1ccc(-c2nc(-c3ccccc3)nc(-c3cccc(-n4c5ccccc5c5ccc(-c6ccc7c(c6)oc6ccccc67)cc54)n3)n2)cc1]

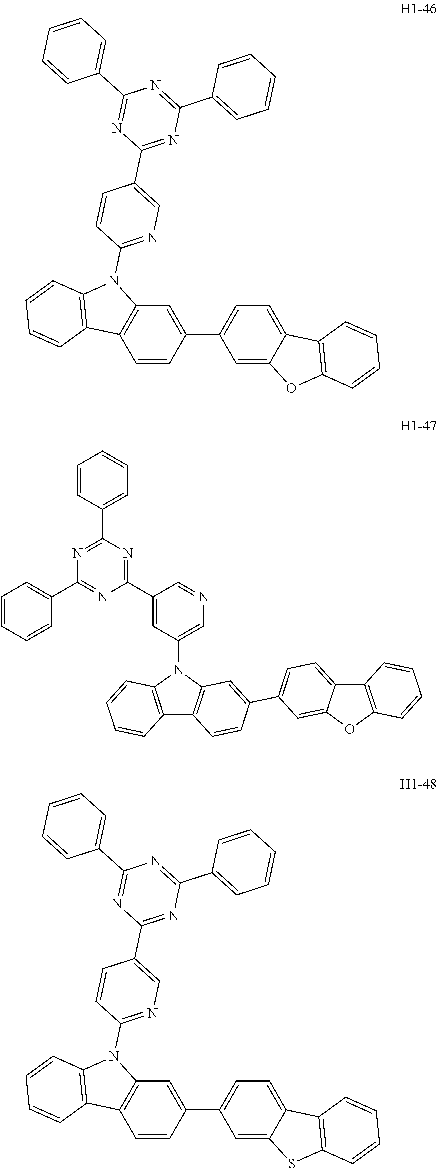 [SMILES: c1ccc(-c2nc(-c3ccccc3)nc(-c3ccc(-n4c5ccccc5c5ccc(-c6ccc7c(c6)oc6ccccc67)cc54)nc3)n2)cc1 ; c1ccc(-c2nc(-c3ccccc3)nc(-c3cncc(-n4c5ccccc5c5ccc(-c6ccc7c(c6)oc6ccccc67)cc54)c3)n2)cc1 ; c1ccc(-c2nc(-c3ccccc3)nc(-c3ccc(-n4c5ccccc5c5ccc(-c6ccc7c(c6)sc6ccccc67)cc54)nc3)n2)cc1]

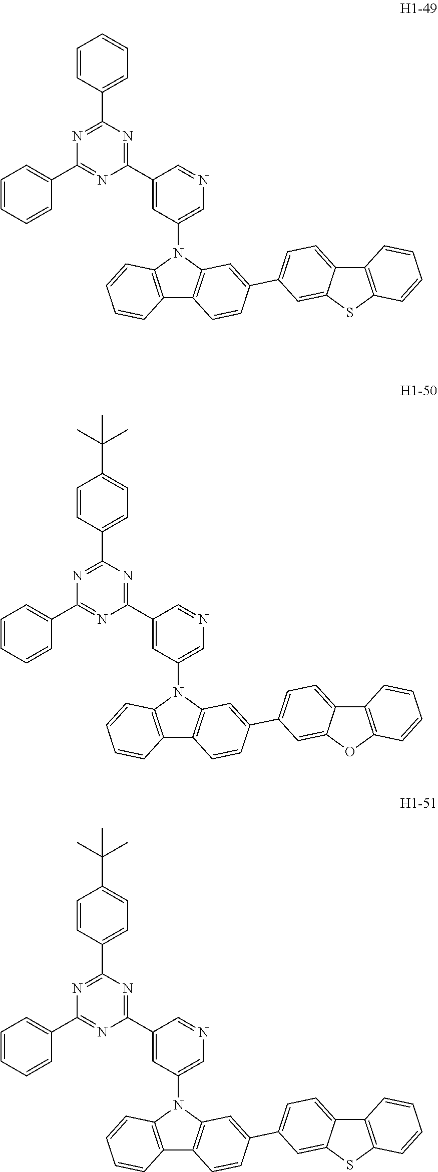 [SMILES: c1ccc(-c2nc(-c3ccccc3)nc(-c3cncc(-n4c5ccccc5c5ccc(-c6ccc7c(c6)sc6ccccc67)cc54)c3)n2)cc1 ; CC(C)(C)c1ccc(-c2nc(-c3ccccc3)nc(-c3cncc(-n4c5ccccc5c5ccc(-c6ccc7c(c6)oc6ccccc67)cc54)c3)n2)cc1 ; CC(C)(C)c1ccc(-c2nc(-c3ccccc3)nc(-c3cncc(-n4c5ccccc5c5ccc(-c6ccc7c(c6)sc6ccccc67)cc54)c3)n2)cc1]

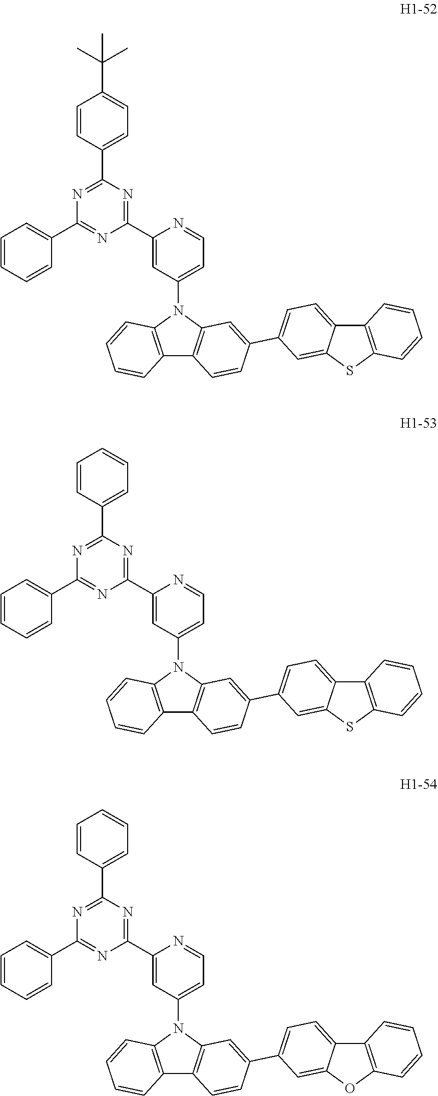 [SMILES: CC(C)(C)c1ccc(-c2nc(-c3ccccc3)nc(-c3cc(-n4c5ccccc5c5ccc(-c6ccc7c(c6)sc6ccccc67)cc54)ccn3)n2)cc1 ; c1ccc(-c2nc(-c3ccccc3)nc(-c3cc(-n4c5ccccc5c5ccc(-c6ccc7c(c6)sc6ccccc67)cc54)ccn3)n2)cc1 ; c1ccc(-c2nc(-c3ccccc3)nc(-c3cc(-n4c5ccccc5c5ccc(-c6ccc7c(c6)oc6ccccc67)cc54)ccn3)n2)cc1]

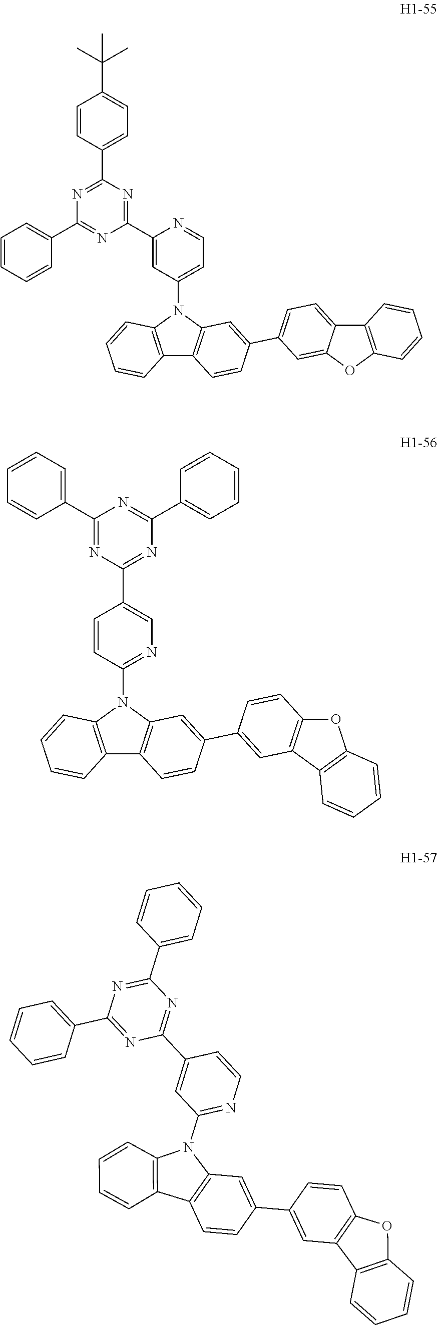 [SMILES: CC(C)(C)c1ccc(-c2nc(-c3ccccc3)nc(-c3cc(-n4c5ccccc5c5ccc(-c6ccc7c(c6)oc6ccccc67)cc54)ccn3)n2)cc1 ; c1ccc(-c2nc(-c3ccccc3)nc(-c3ccc(-n4c5ccccc5c5ccc(-c6ccc7oc8ccccc8c7c6)cc54)nc3)n2)cc1 ; c1ccc(-c2nc(-c3ccccc3)nc(-c3ccnc(-n4c5ccccc5c5ccc(-c6ccc7oc8ccccc8c7c6)cc54)c3)n2)cc1]

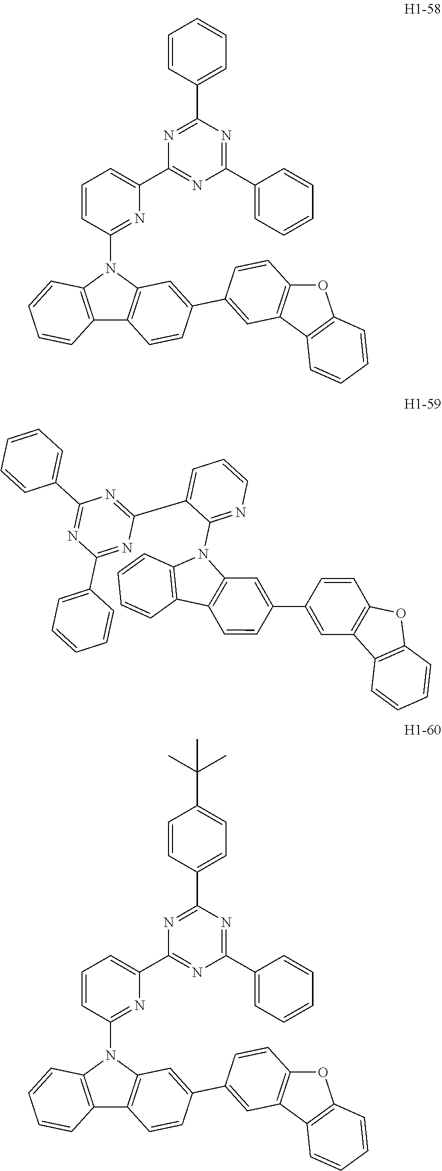 [SMILES: c1ccc(-c2nc(-c3ccccc3)nc(-c3cccc(-n4c5ccccc5c5ccc(-c6ccc7oc8ccccc8c7c6)cc54)n3)n2)cc1 ; c1ccc(-c2nc(-c3ccccc3)nc(-c3cccnc3-n3c4ccccc4c4ccc(-c5ccc6oc7ccccc7c6c5)cc43)n2)cc1 ; CC(C)(C)c1ccc(-c2nc(-c3ccccc3)nc(-c3cccc(-n4c5ccccc5c5ccc(-c6ccc7oc8ccccc8c7c6)cc54)n3)n2)cc1]

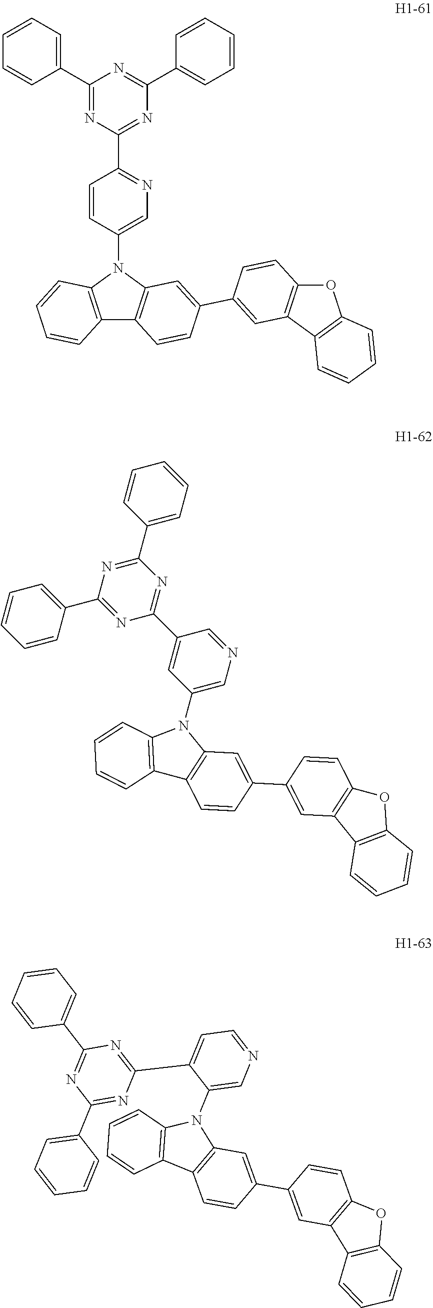 [SMILES: c1ccc(-c2nc(-c3ccccc3)nc(-c3ccc(-n4c5ccccc5c5ccc(-c6ccc7oc8ccccc8c7c6)cc54)cn3)n2)cc1 ; c1ccc(-c2nc(-c3ccccc3)nc(-c3cncc(-n4c5ccccc5c5ccc(-c6ccc7oc8ccccc8c7c6)cc54)c3)n2)cc1 ; c1ccc(-c2nc(-c3ccccc3)nc(-c3ccncc3-n3c4ccccc4c4ccc(-c5ccc6oc7ccccc7c6c5)cc43)n2)cc1]

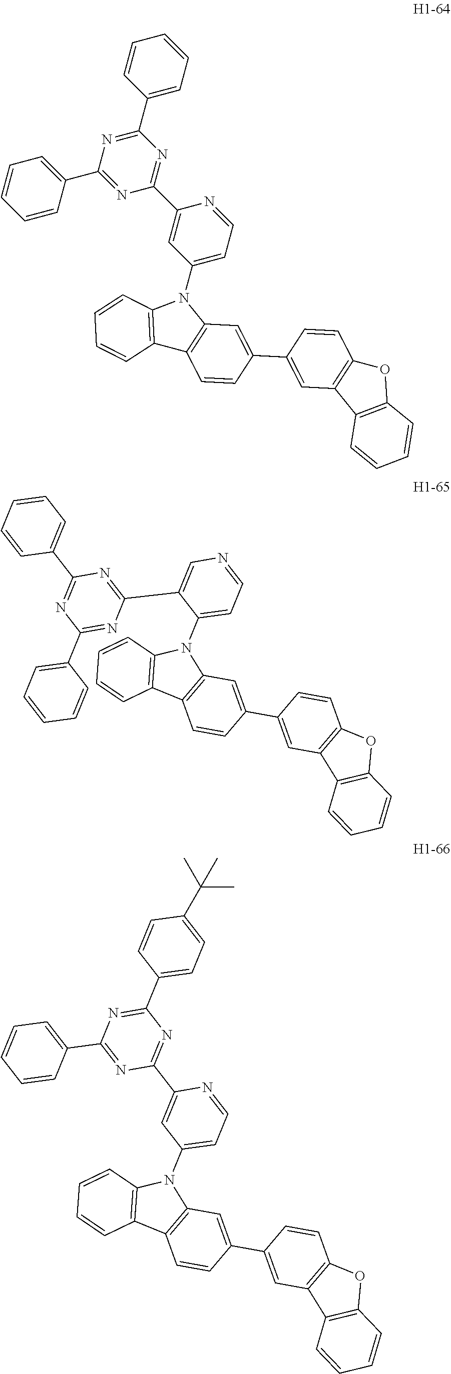 [SMILES: c1ccc(-c2nc(-c3ccccc3)nc(-c3cc(-n4c5ccccc5c5ccc(-c6ccc7oc8ccccc8c7c6)cc54)ccn3)n2)cc1 ; c1ccc(-c2nc(-c3ccccc3)nc(-c3cnccc3-n3c4ccccc4c4ccc(-c5ccc6oc7ccccc7c6c5)cc43)n2)cc1 ; CC(C)(C)c1ccc(-c2nc(-c3ccccc3)nc(-c3cc(-n4c5ccccc5c5ccc(-c6ccc7oc8ccccc8c7c6)cc54)ccn3)n2)cc1]

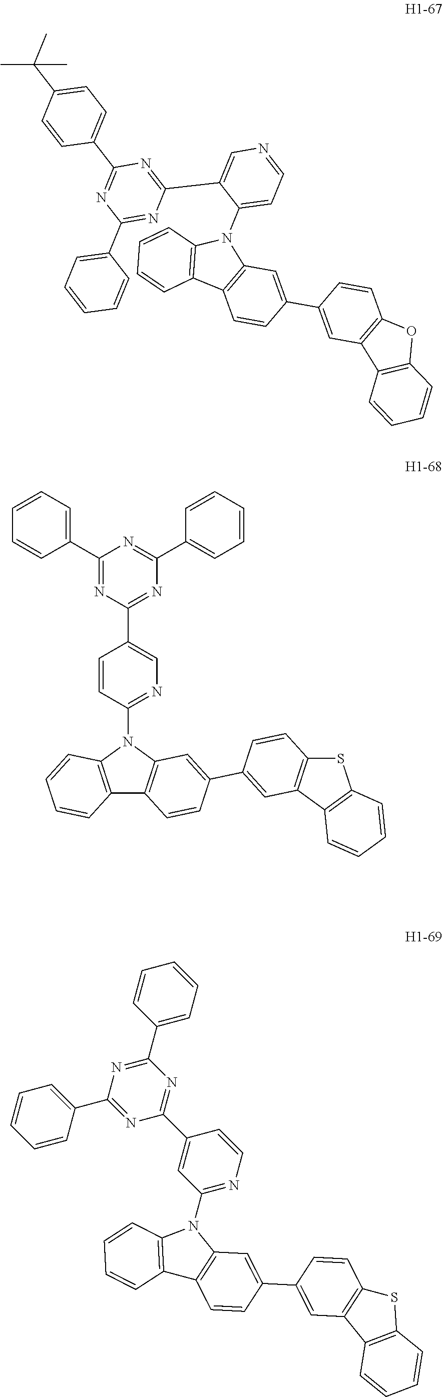 [SMILES: CC(C)(C)c1ccc(-c2nc(-c3ccccc3)nc(-c3cnccc3-n3c4ccccc4c4ccc(-c5ccc6oc7ccccc7c6c5)cc43)n2)cc1 ; c1ccc(-c2nc(-c3ccccc3)nc(-c3ccc(-n4c5ccccc5c5ccc(-c6ccc7sc8ccccc8c7c6)cc54)nc3)n2)cc1 ; c1ccc(-c2nc(-c3ccccc3)nc(-c3ccnc(-n4c5ccccc5c5ccc(-c6ccc7sc8ccccc8c7c6)cc54)c3)n2)cc1]

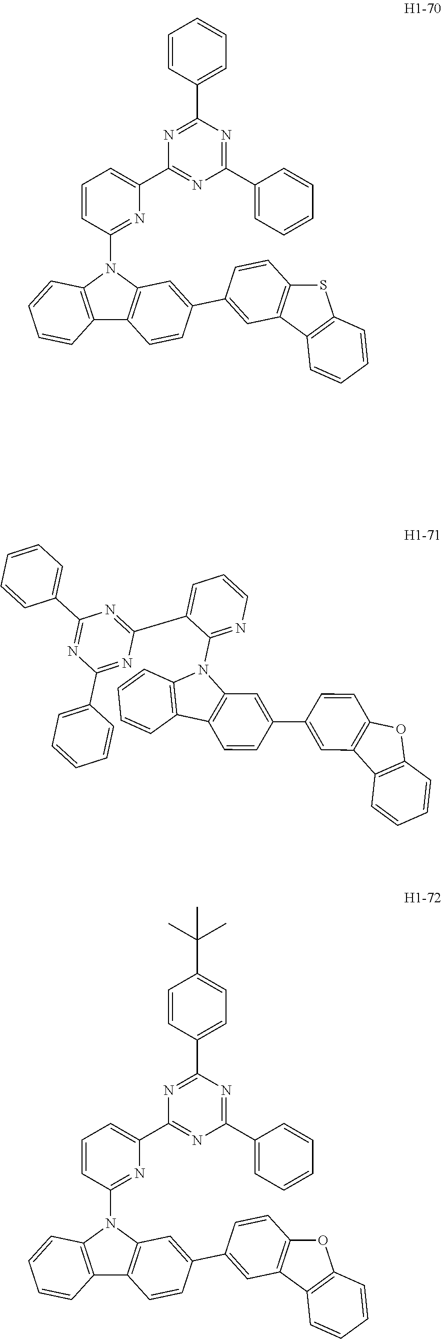 [SMILES: c1ccc(-c2nc(-c3ccccc3)nc(-c3cccc(-n4c5ccccc5c5ccc(-c6ccc7sc8ccccc8c7c6)cc54)n3)n2)cc1 ; c1ccc(-c2nc(-c3ccccc3)nc(-c3cccnc3-n3c4ccccc4c4ccc(-c5ccc6oc7ccccc7c6c5)cc43)n2)cc1 ; CC(C)(C)c1ccc(-c2nc(-c3ccccc3)nc(-c3cccc(-n4c5ccccc5c5ccc(-c6ccc7oc8ccccc8c7c6)cc54)n3)n2)cc1]

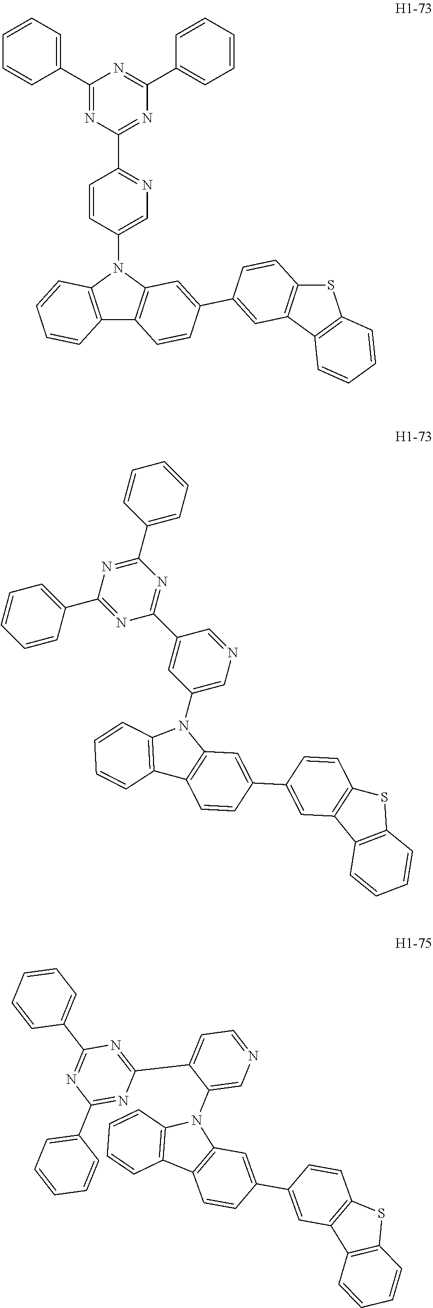 [SMILES: c1ccc(-c2nc(-c3ccccc3)nc(-c3ccc(-n4c5ccccc5c5ccc(-c6ccc7sc8ccccc8c7c6)cc54)cn3)n2)cc1 ; c1ccc(-c2nc(-c3ccccc3)nc(-c3cncc(-n4c5ccccc5c5ccc(-c6ccc7sc8ccccc8c7c6)cc54)c3)n2)cc1 ; c1ccc(-c2nc(-c3ccccc3)nc(-c3ccncc3-n3c4ccccc4c4ccc(-c5ccc6sc7ccccc7c6c5)cc43)n2)cc1]

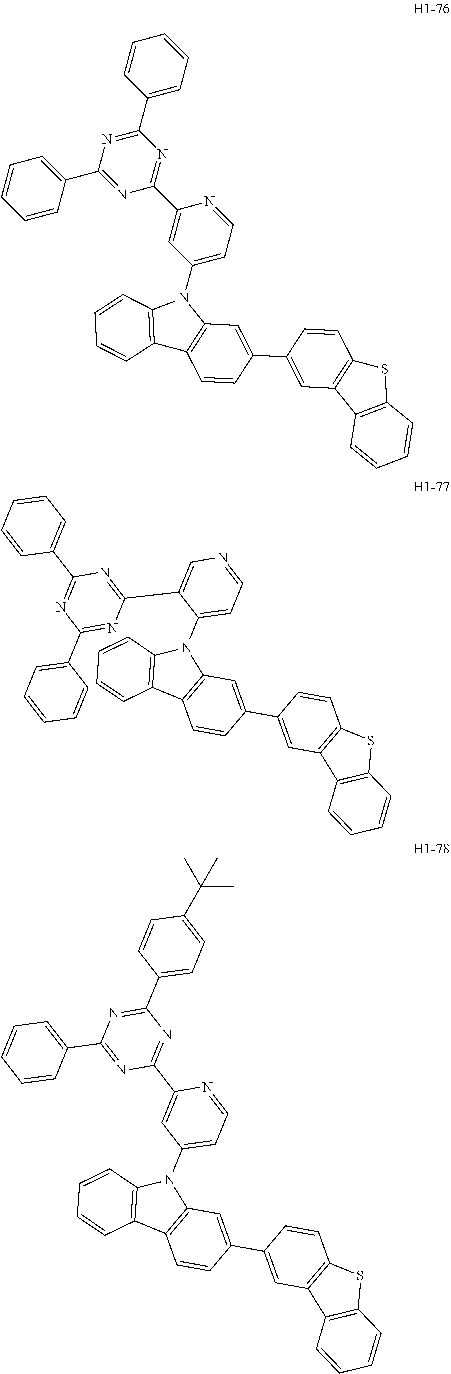 [SMILES: c1ccc(-c2nc(-c3ccccc3)nc(-c3cc(-n4c5ccccc5c5ccc(-c6ccc7sc8ccccc8c7c6)cc54)ccn3)n2)cc1 ; c1ccc(-c2nc(-c3ccccc3)nc(-c3cnccc3-n3c4ccccc4c4ccc(-c5ccc6sc7ccccc7c6c5)cc43)n2)cc1 ; CC(C)(C)c1ccc(-c2nc(-c3ccccc3)nc(-c3cc(-n4c5ccccc5c5ccc(-c6ccc7sc8ccccc8c7c6)cc54)ccn3)n2)cc1]

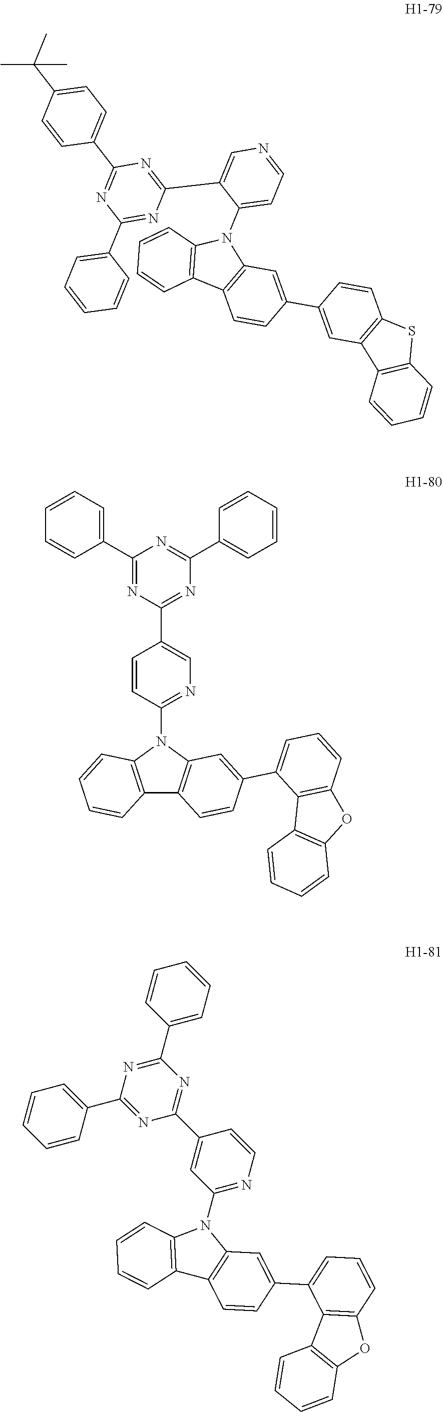 [SMILES: CC(C)(C)c1ccc(-c2nc(-c3ccccc3)nc(-c3cnccc3-n3c4ccccc4c4ccc(-c5ccc6sc7ccccc7c6c5)cc43)n2)cc1 ; c1ccc(-c2nc(-c3ccccc3)nc(-c3ccc(-n4c5ccccc5c5ccc(-c6cccc7oc8ccccc8c67)cc54)nc3)n2)cc1 ; c1ccc(-c2nc(-c3ccccc3)nc(-c3ccnc(-n4c5ccccc5c5ccc(-c6cccc7oc8ccccc8c67)cc54)c3)n2)cc1]

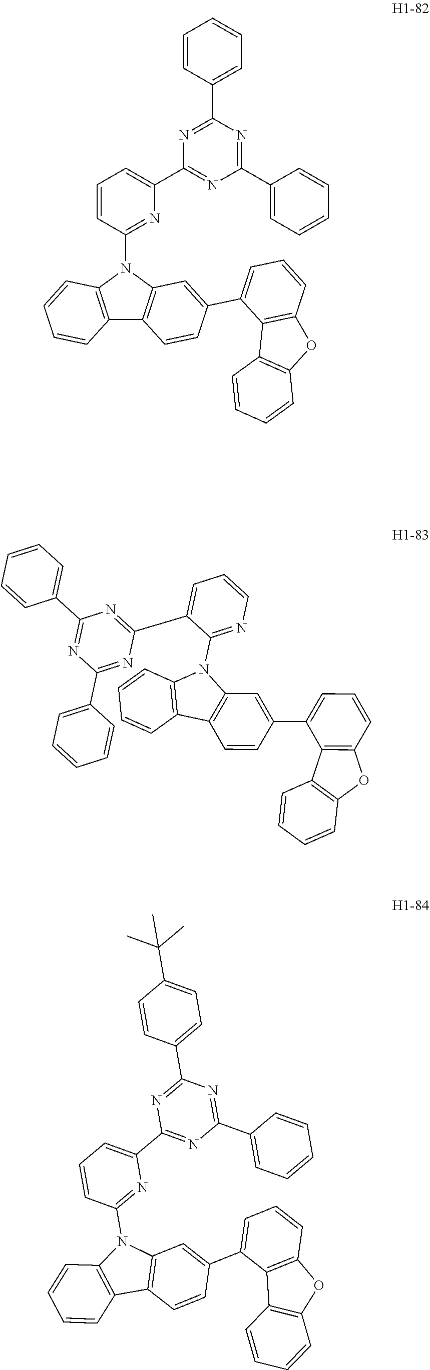 [SMILES: c1ccc(-c2nc(-c3ccccc3)nc(-c3cccc(-n4c5ccccc5c5ccc(-c6cccc7oc8ccccc8c67)cc54)n3)n2)cc1 ; c1ccc(-c2nc(-c3ccccc3)nc(-c3cccnc3-n3c4ccccc4c4ccc(-c5cccc6oc7ccccc7c56)cc43)n2)cc1 ; CC(C)(C)c1ccc(-c2nc(-c3ccccc3)nc(-c3cccc(-n4c5ccccc5c5ccc(-c6cccc7oc8ccccc8c67)cc54)n3)n2)cc1]

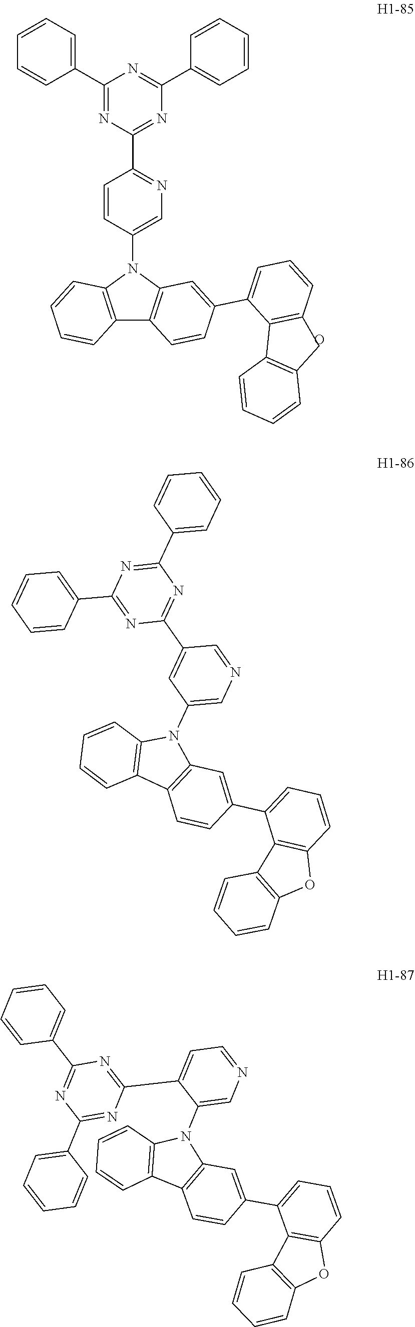

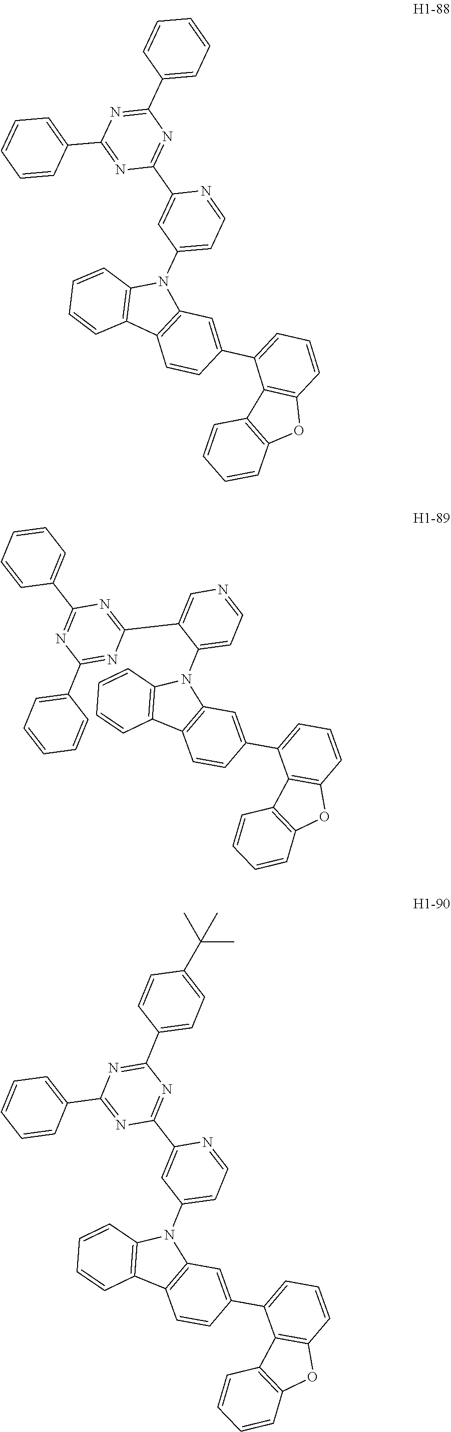 [SMILES: c1ccc(-c2nc(-c3ccccc3)nc(-c3cc(-n4c5ccccc5c5ccc(-c6cccc7oc8ccccc8c67)cc54)ccn3)n2)cc1 ; c1ccc(-c2nc(-c3ccccc3)nc(-c3cnccc3-n3c4ccccc4c4ccc(-c5cccc6oc7ccccc7c56)cc43)n2)cc1 ; CC(C)(C)c1ccc(-c2nc(-c3ccccc3)nc(-c3cc(-n4c5ccccc5c5ccc(-c6cccc7oc8ccccc8c67)cc54)ccn3)n2)cc1]

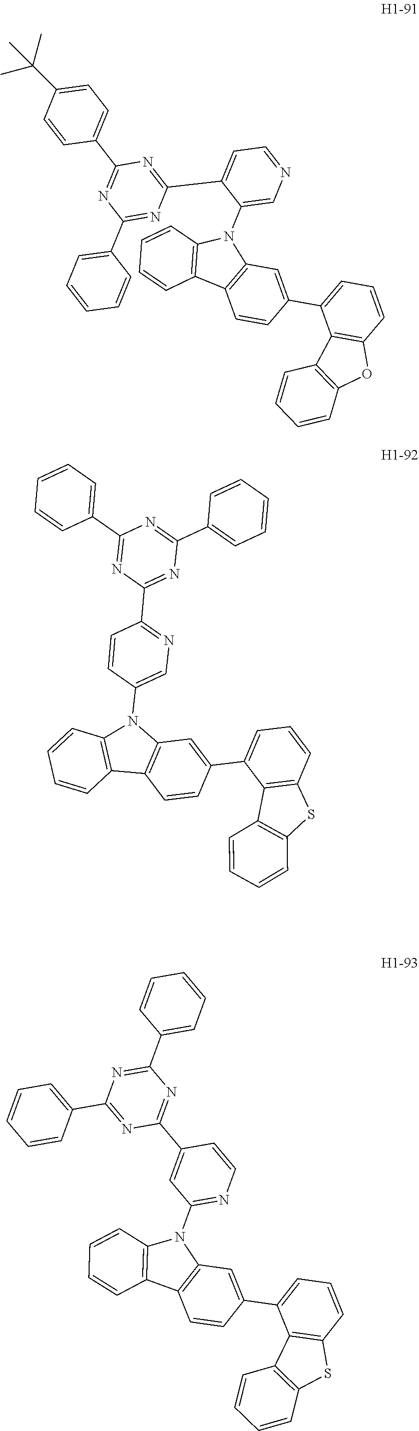 [SMILES: CC(C)(C)c1ccc(-c2nc(-c3ccccc3)nc(-c3ccncc3-n3c4ccccc4c4ccc(-c5cccc6oc7ccccc7c56)cc43)n2)cc1 ; c1ccc(-c2nc(-c3ccccc3)nc(-c3ccc(-n4c5ccccc5c5ccc(-c6cccc7sc8ccccc8c67)cc54)cn3)n2)cc1 ; c1ccc(-c2nc(-c3ccccc3)nc(-c3ccnc(-n4c5ccccc5c5ccc(-c6cccc7sc8ccccc8c67)cc54)c3)n2)cc1]

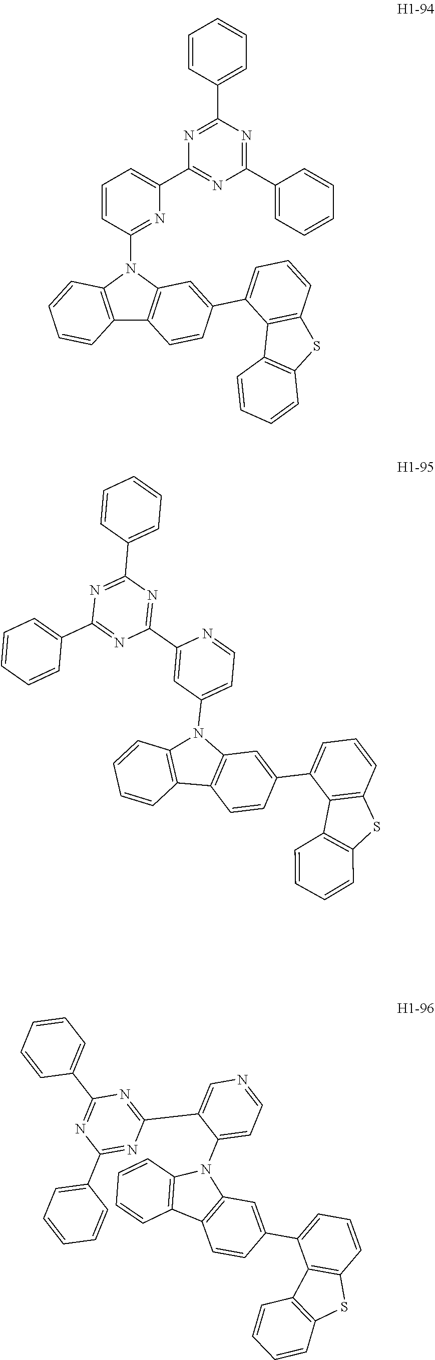 [SMILES: c1ccc(-c2nc(-c3ccccc3)nc(-c3cccc(-n4c5ccccc5c5ccc(-c6cccc7sc8ccccc8c67)cc54)n3)n2)cc1 ; c1ccc(-c2nc(-c3ccccc3)nc(-c3cc(-n4c5ccccc5c5ccc(-c6cccc7sc8ccccc8c67)cc54)ccn3)n2)cc1 ; c1ccc(-c2nc(-c3ccccc3)nc(-c3cnccc3-n3c4ccccc4c4ccc(-c5cccc6sc7ccccc7c56)cc43)n2)cc1]

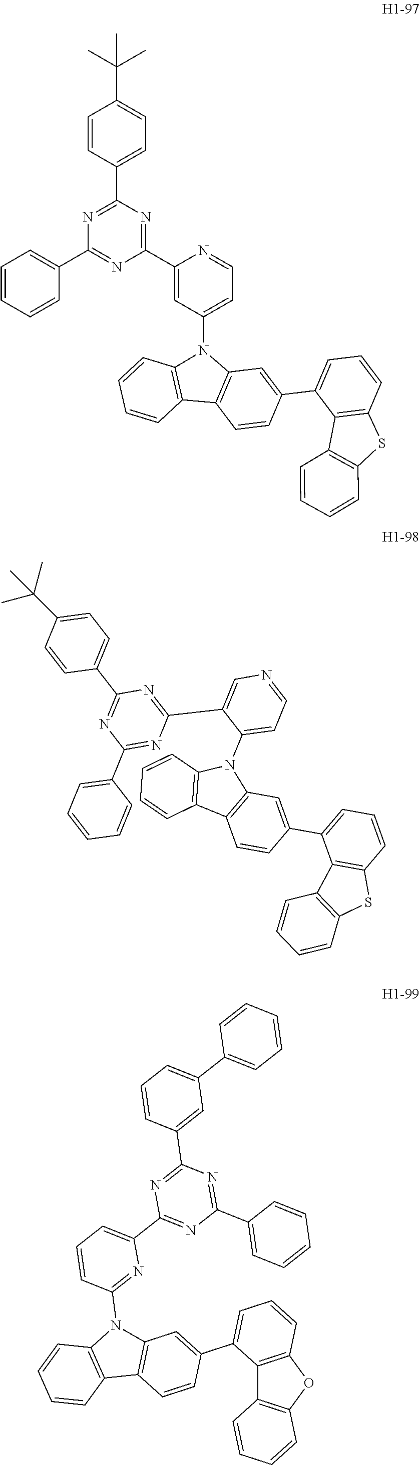 [SMILES: CC(C)(C)c1ccc(-c2nc(-c3ccccc3)nc(-c3cc(-n4c5ccccc5c5ccc(-c6cccc7sc8ccccc8c67)cc54)ccn3)n2)cc1 ; CC(C)(C)c1ccc(-c2nc(-c3ccccc3)nc(-c3cnccc3-n3c4ccccc4c4ccc(-c5cccc6sc7ccccc7c56)cc43)n2)cc1 ; c1ccc(-c2cccc(-c3nc(-c4ccccc4)nc(-c4cccc(-n5c6ccccc6c6ccc(-c7cccc8oc9ccccc9c78)cc65)n4)n3)c2)cc1]

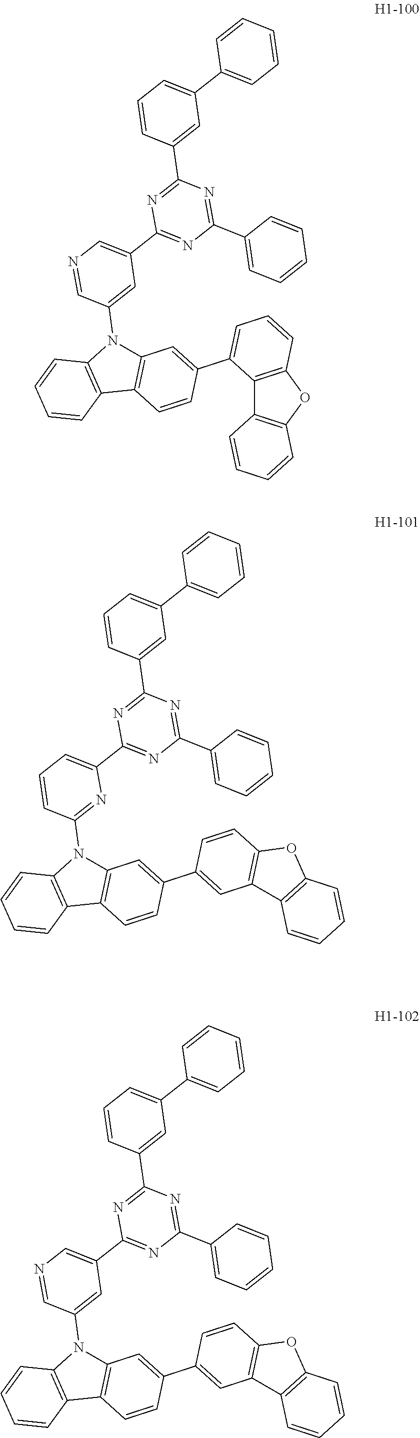 [SMILES: c1ccc(-c2cccc(-c3nc(-c4ccccc4)nc(-c4cncc(-n5c6ccccc6c6ccc(-c7cccc8oc9ccccc9c78)cc65)c4)n3)c2)cc1 ; c1ccc(-c2cccc(-c3nc(-c4ccccc4)nc(-c4cccc(-n5c6ccccc6c6ccc(-c7ccc8oc9ccccc9c8c7)cc65)n4)n3)c2)cc1 ; c1ccc(-c2cccc(-c3nc(-c4ccccc4)nc(-c4cncc(-n5c6ccccc6c6ccc(-c7ccc8oc9ccccc9c8c7)cc65)c4)n3)c2)cc1]

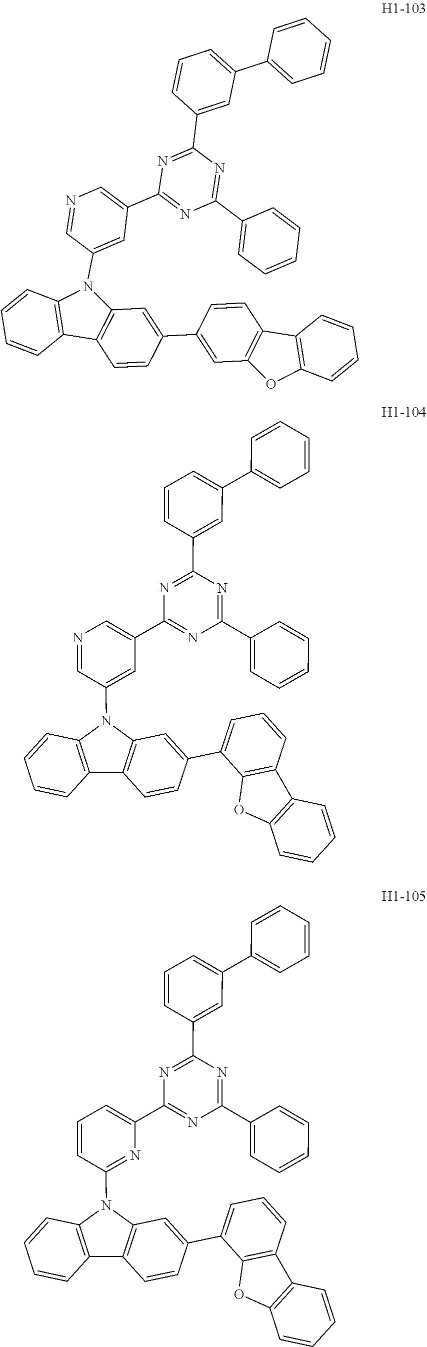

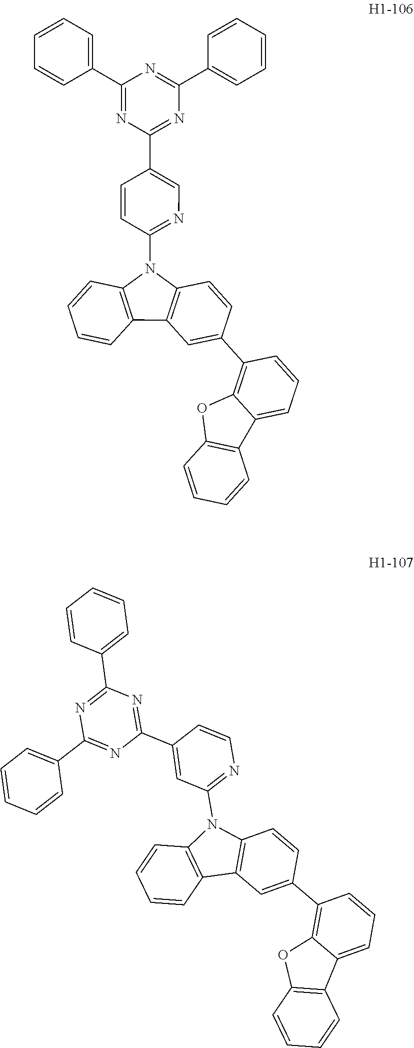 [SMILES: c1ccc(-c2nc(-c3ccccc3)nc(-c3ccc(-n4c5ccccc5c5cc(-c6cccc7c6oc6ccccc67)ccc54)nc3)n2)cc1 ; c1ccc(-c2nc(-c3ccccc3)nc(-c3ccnc(-n4c5ccccc5c5cc(-c6cccc7c6oc6ccccc67)ccc54)c3)n2)cc1]

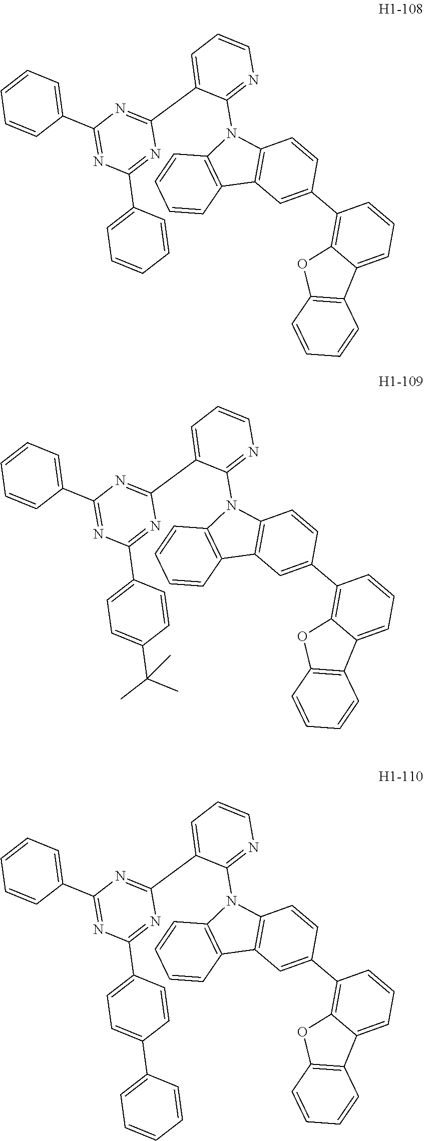 [SMILES: c1ccc(-c2nc(-c3ccccc3)nc(-c3cccnc3-n3c4ccccc4c4cc(-c5cccc6c5oc5ccccc56)ccc43)n2)cc1 ; CC(C)(C)c1ccc(-c2nc(-c3ccccc3)nc(-c3cccnc3-n3c4ccccc4c4cc(-c5cccc6c5oc5ccccc56)ccc43)n2)cc1 ; c1ccc(-c2ccc(-c3nc(-c4ccccc4)nc(-c4cccnc4-n4c5ccccc5c5cc(-c6cccc7c6oc6ccccc67)ccc54)n3)cc2)cc1]

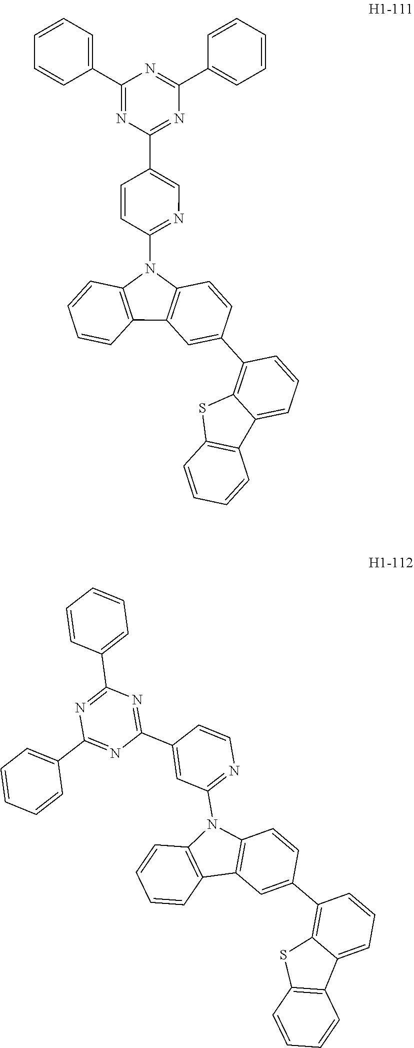 [SMILES: c1ccc(-c2nc(-c3ccccc3)nc(-c3ccc(-n4c5ccccc5c5cc(-c6cccc7c6sc6ccccc67)ccc54)nc3)n2)cc1 ; c1ccc(-c2nc(-c3ccccc3)nc(-c3ccnc(-n4c5ccccc5c5cc(-c6cccc7c6sc6ccccc67)ccc54)c3)n2)cc1]

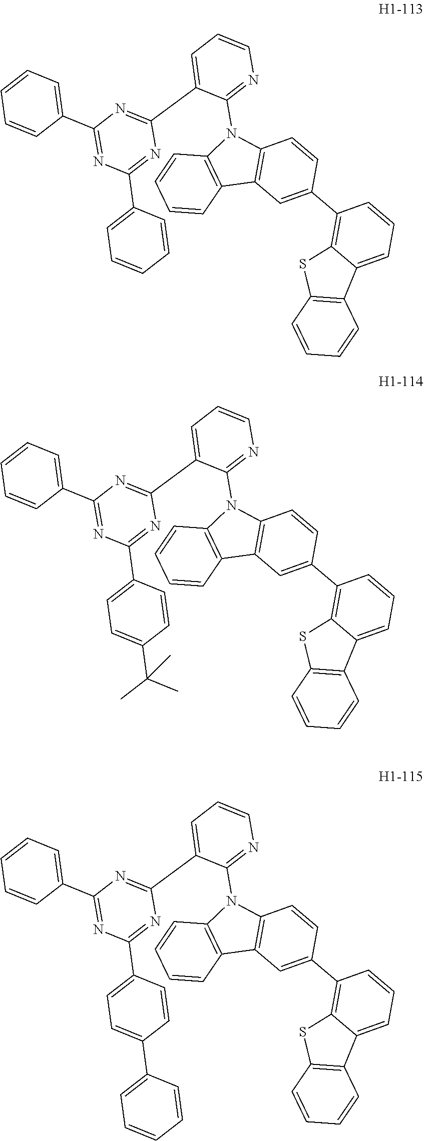 [SMILES: c1ccc(-c2nc(-c3ccccc3)nc(-c3cccnc3-n3c4ccccc4c4cc(-c5cccc6c5sc5ccccc56)ccc43)n2)cc1 ; CC(C)(C)c1ccc(-c2nc(-c3ccccc3)nc(-c3cccnc3-n3c4ccccc4c4cc(-c5cccc6c5sc5ccccc56)ccc43)n2)cc1 ; c1ccc(-c2ccc(-c3nc(-c4ccccc4)nc(-c4cccnc4-n4c5ccccc5c5cc(-c6cccc7c6sc6ccccc67)ccc54)n3)cc2)cc1]

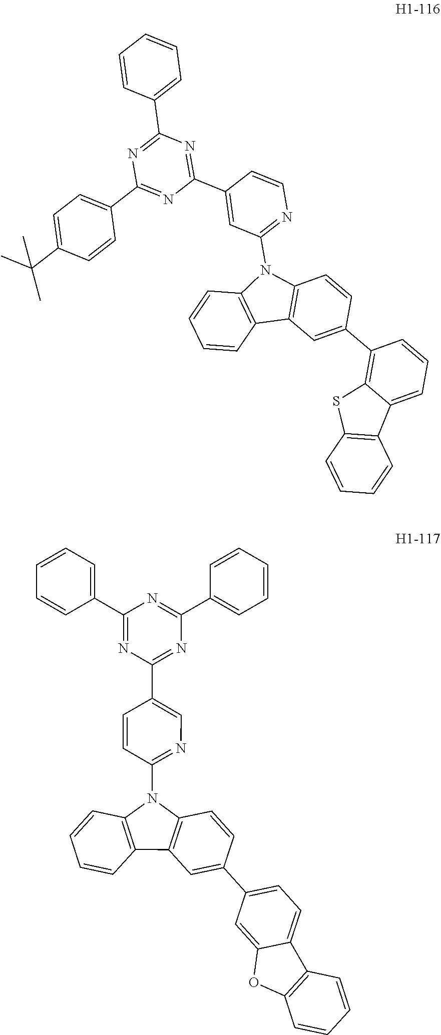 [SMILES: CC(C)(C)c1ccc(-c2nc(-c3ccccc3)nc(-c3ccnc(-n4c5ccccc5c5cc(-c6cccc7c6sc6ccccc67)ccc54)c3)n2)cc1 ; c1ccc(-c2nc(-c3ccccc3)nc(-c3ccc(-n4c5ccccc5c5cc(-c6ccc7c(c6)oc6ccccc67)ccc54)nc3)n2)cc1]

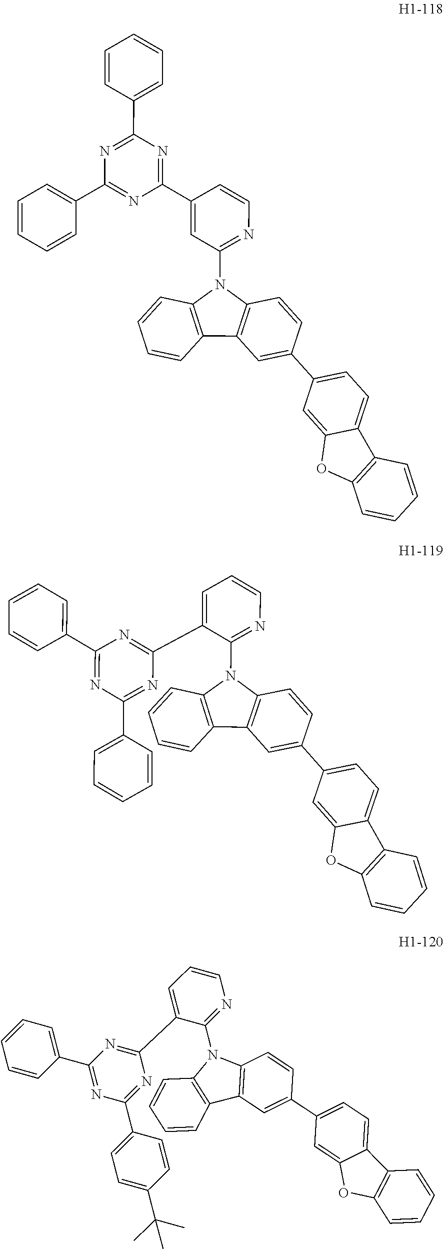 [SMILES: c1ccc(-c2nc(-c3ccccc3)nc(-c3ccnc(-n4c5ccccc5c5cc(-c6ccc7c(c6)oc6ccccc67)ccc54)c3)n2)cc1 ; c1ccc(-c2nc(-c3ccccc3)nc(-c3cccnc3-n3c4ccccc4c4cc(-c5ccc6c(c5)oc5ccccc56)ccc43)n2)cc1 ; CC(C)(C)c1ccc(-c2nc(-c3ccccc3)nc(-c3cccnc3-n3c4ccccc4c4cc(-c5ccc6c(c5)oc5ccccc56)ccc43)n2)cc1]

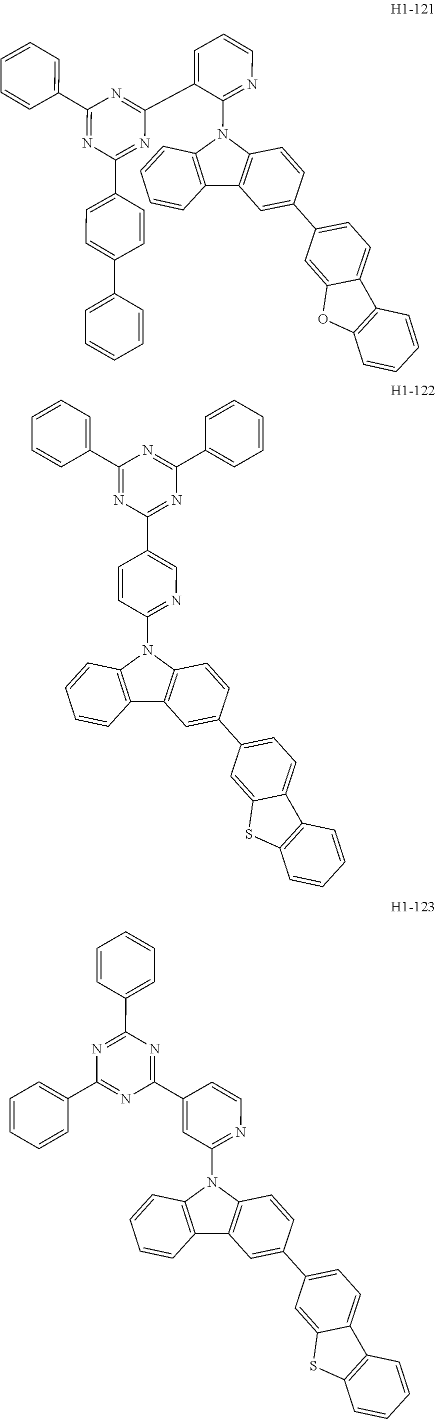 [SMILES: c1ccc(-c2ccc(-c3nc(-c4ccccc4)nc(-c4cccnc4-n4c5ccccc5c5cc(-c6ccc7c(c6)oc6ccccc67)ccc54)n3)cc2)cc1 ; c1ccc(-c2nc(-c3ccccc3)nc(-c3ccc(-n4c5ccccc5c5cc(-c6ccc7c(c6)sc6ccccc67)ccc54)nc3)n2)cc1 ; c1ccc(-c2nc(-c3ccccc3)nc(-c3ccnc(-n4c5ccccc5c5cc(-c6ccc7c(c6)sc6ccccc67)ccc54)c3)n2)cc1]

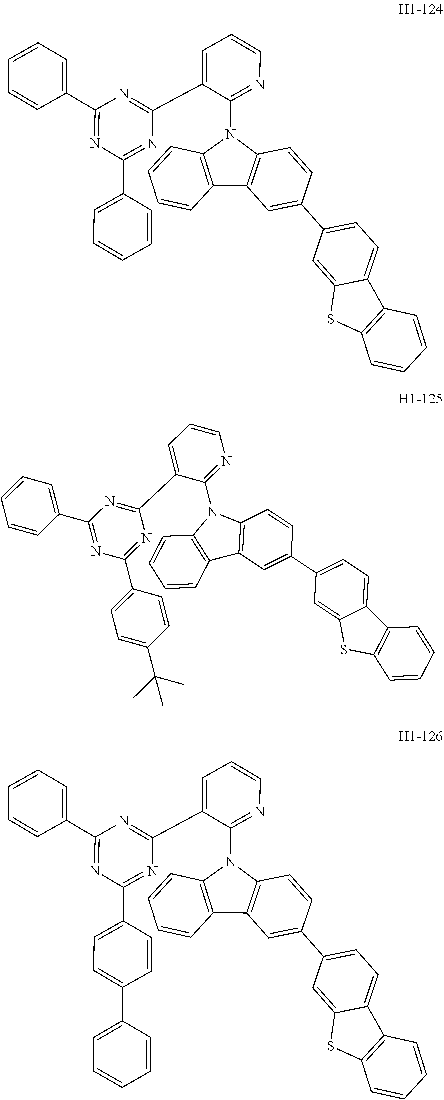 [SMILES: c1ccc(-c2nc(-c3ccccc3)nc(-c3cccnc3-n3c4ccccc4c4cc(-c5ccc6c(c5)sc5ccccc56)ccc43)n2)cc1 ; CC(C)(C)c1ccc(-c2nc(-c3ccccc3)nc(-c3cccnc3-n3c4ccccc4c4cc(-c5ccc6c(c5)sc5ccccc56)ccc43)n2)cc1 ; c1ccc(-c2ccc(-c3nc(-c4ccccc4)nc(-c4cccnc4-n4c5ccccc5c5cc(-c6ccc7c(c6)sc6ccccc67)ccc54)n3)cc2)cc1]

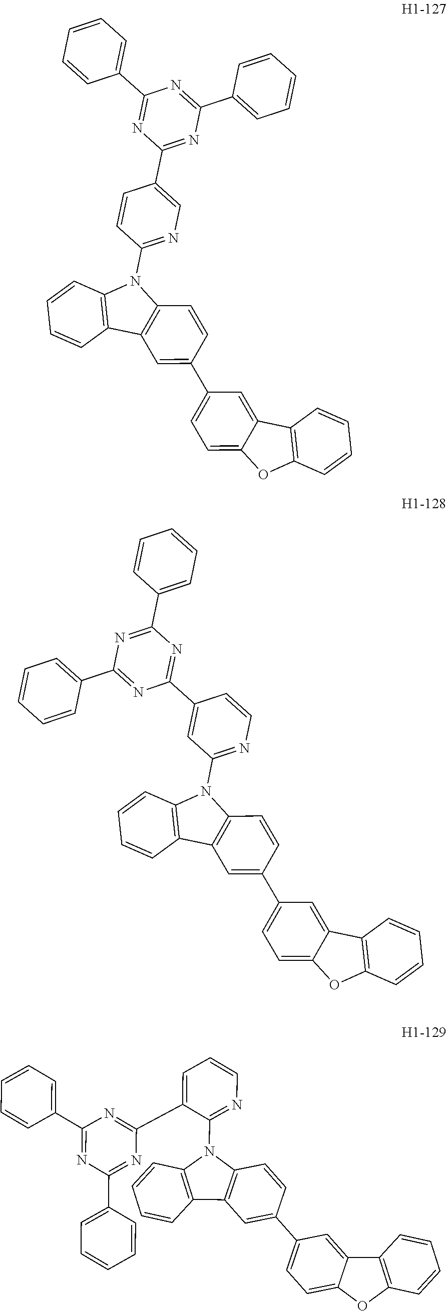 [SMILES: c1ccc(-c2nc(-c3ccccc3)nc(-c3ccc(-n4c5ccccc5c5cc(-c6ccc7oc8ccccc8c7c6)ccc54)nc3)n2)cc1 ; c1ccc(-c2nc(-c3ccccc3)nc(-c3ccnc(-n4c5ccccc5c5cc(-c6ccc7oc8ccccc8c7c6)ccc54)c3)n2)cc1 ; c1ccc(-c2nc(-c3ccccc3)nc(-c3cccnc3-n3c4ccccc4c4cc(-c5ccc6oc7ccccc7c6c5)ccc43)n2)cc1]

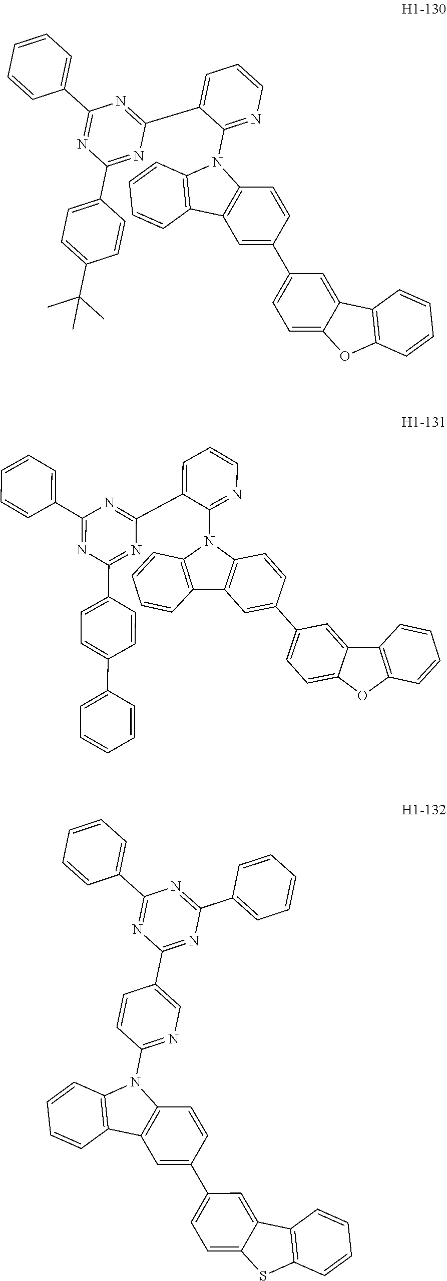 [SMILES: CC(C)(C)c1ccc(-c2nc(-c3ccccc3)nc(-c3cccnc3-n3c4ccccc4c4cc(-c5ccc6oc7ccccc7c6c5)ccc43)n2)cc1 ; c1ccc(-c2ccc(-c3nc(-c4ccccc4)nc(-c4cccnc4-n4c5ccccc5c5cc(-c6ccc7oc8ccccc8c7c6)ccc54)n3)cc2)cc1 ; c1ccc(-c2nc(-c3ccccc3)nc(-c3ccc(-n4c5ccccc5c5cc(-c6ccc7sc8ccccc8c7c6)ccc54)nc3)n2)cc1]

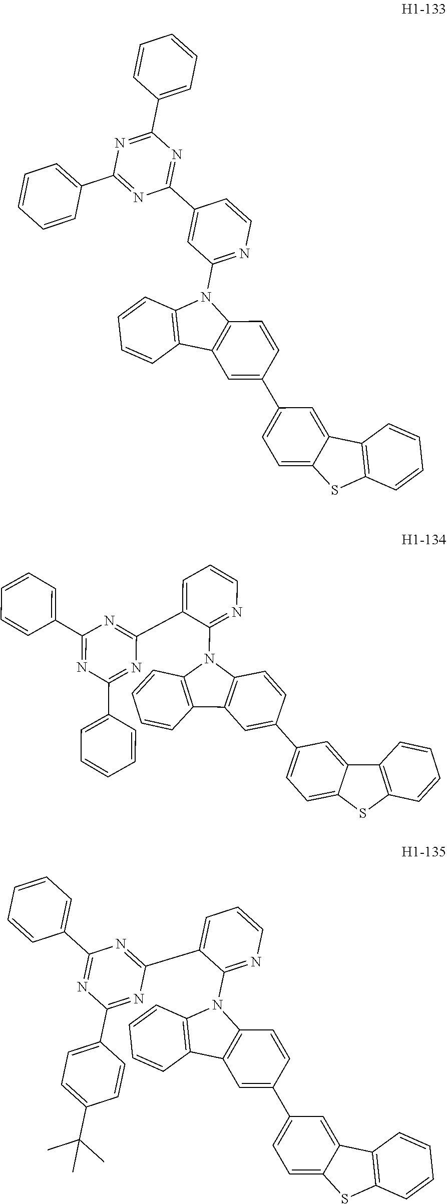 [SMILES: c1ccc(-c2nc(-c3ccccc3)nc(-c3ccnc(-n4c5ccccc5c5cc(-c6ccc7sc8ccccc8c7c6)ccc54)c3)n2)cc1 ; c1ccc(-c2nc(-c3ccccc3)nc(-c3cccnc3-n3c4ccccc4c4cc(-c5ccc6sc7ccccc7c6c5)ccc43)n2)cc1 ; CC(C)(C)c1ccc(-c2nc(-c3ccccc3)nc(-c3cccnc3-n3c4ccccc4c4cc(-c5ccc6sc7ccccc7c6c5)ccc43)n2)cc1]

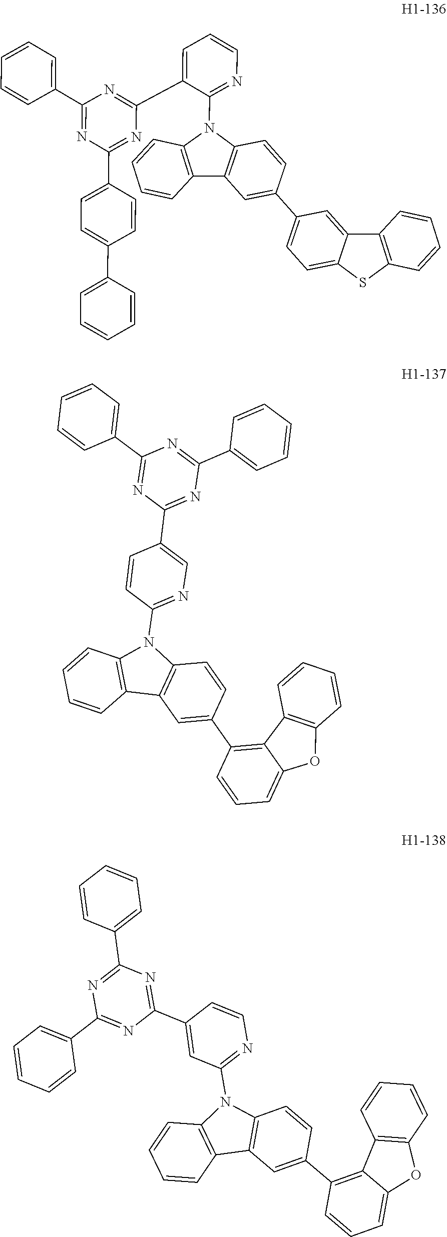 [SMILES: c1ccc(-c2ccc(-c3nc(-c4ccccc4)nc(-c4cccnc4-n4c5ccccc5c5cc(-c6ccc7sc8ccccc8c7c6)ccc54)n3)cc2)cc1 ; c1ccc(-c2nc(-c3ccccc3)nc(-c3ccc(-n4c5ccccc5c5cc(-c6cccc7oc8ccccc8c67)ccc54)nc3)n2)cc1 ; c1ccc(-c2nc(-c3ccccc3)nc(-c3ccnc(-n4c5ccccc5c5cc(-c6cccc7oc8ccccc8c67)ccc54)c3)n2)cc1]

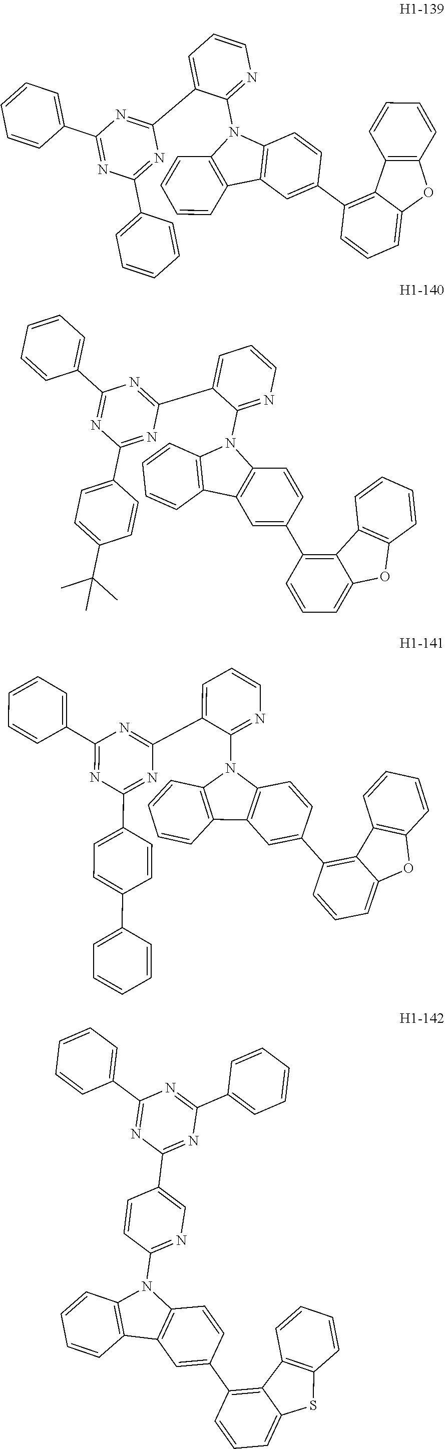

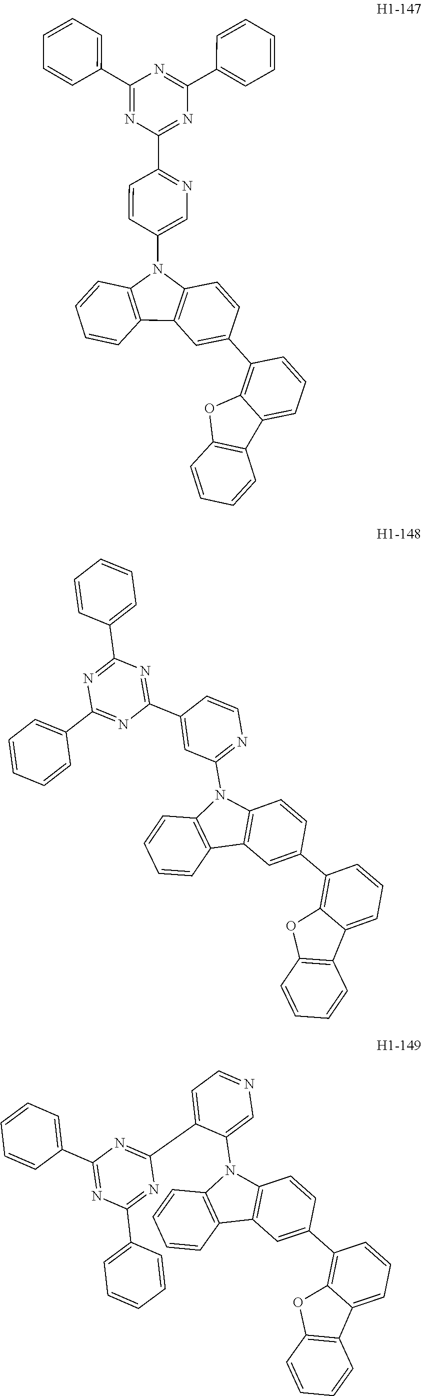 [SMILES: c1ccc(-c2nc(-c3ccccc3)nc(-c3ccc(-n4c5ccccc5c5cc(-c6cccc7c6oc6ccccc67)ccc54)cn3)n2)cc1 ; c1ccc(-c2nc(-c3ccccc3)nc(-c3ccnc(-n4c5ccccc5c5cc(-c6cccc7c6oc6ccccc67)ccc54)c3)n2)cc1 ; c1ccc(-c2nc(-c3ccccc3)nc(-c3ccncc3-n3c4ccccc4c4cc(-c5cccc6c5oc5ccccc56)ccc43)n2)cc1]

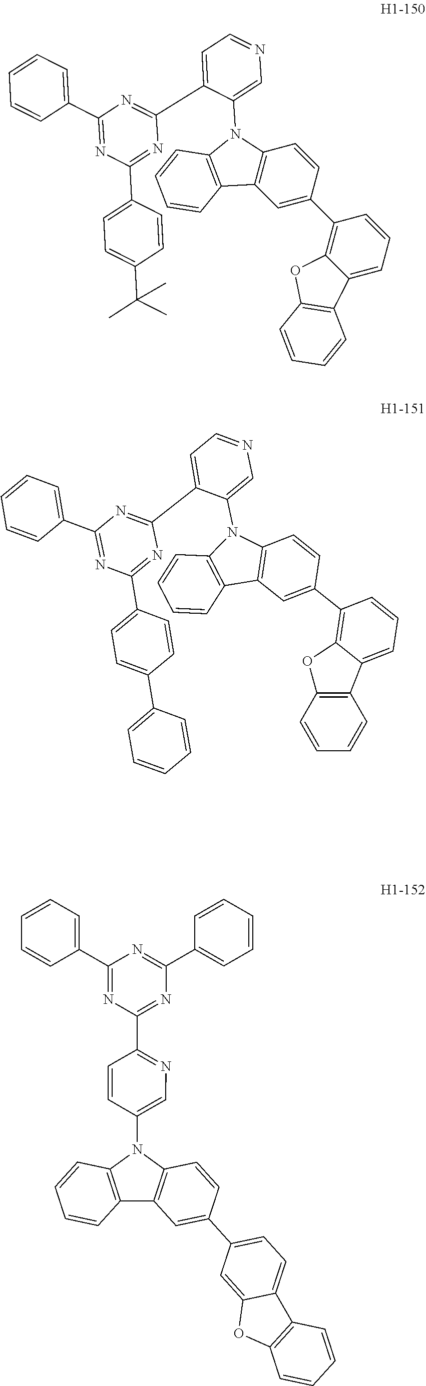 [SMILES: CC(C)(C)c1ccc(-c2nc(-c3ccccc3)nc(-c3ccncc3-n3c4ccccc4c4cc(-c5cccc6c5oc5ccccc56)ccc43)n2)cc1 ; c1ccc(-c2ccc(-c3nc(-c4ccccc4)nc(-c4ccncc4-n4c5ccccc5c5cc(-c6cccc7c6oc6ccccc67)ccc54)n3)cc2)cc1 ; c1ccc(-c2nc(-c3ccccc3)nc(-c3ccc(-n4c5ccccc5c5cc(-c6ccc7c(c6)oc6ccccc67)ccc54)cn3)n2)cc1]

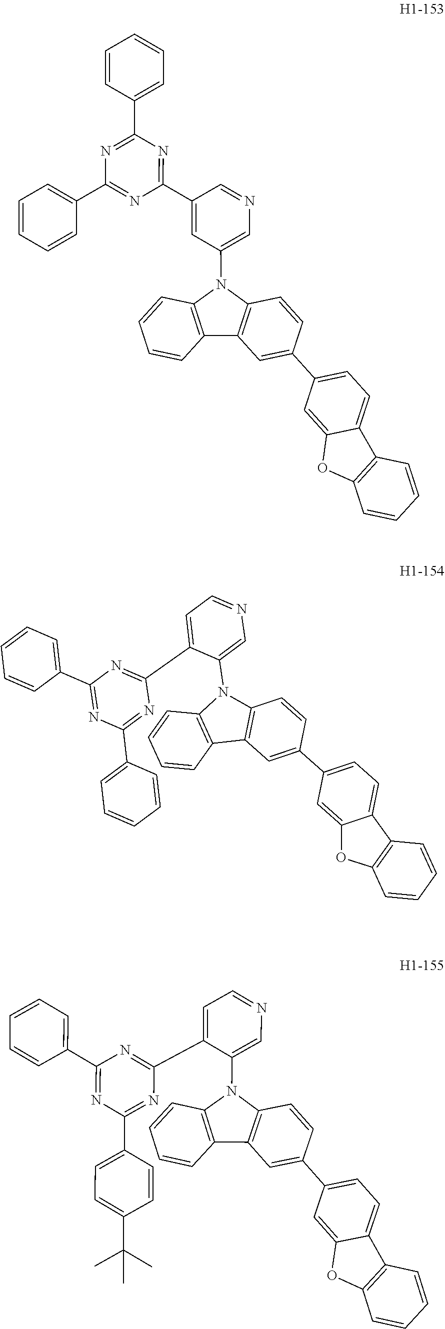 [SMILES: c1ccc(-c2nc(-c3ccccc3)nc(-c3cncc(-n4c5ccccc5c5cc(-c6ccc7c(c6)oc6ccccc67)ccc54)c3)n2)cc1 ; c1ccc(-c2nc(-c3ccccc3)nc(-c3ccncc3-n3c4ccccc4c4cc(-c5ccc6c(c5)oc5ccccc56)ccc43)n2)cc1 ; CC(C)(C)c1ccc(-c2nc(-c3ccccc3)nc(-c3ccncc3-n3c4ccccc4c4cc(-c5ccc6c(c5)oc5ccccc56)ccc43)n2)cc1]

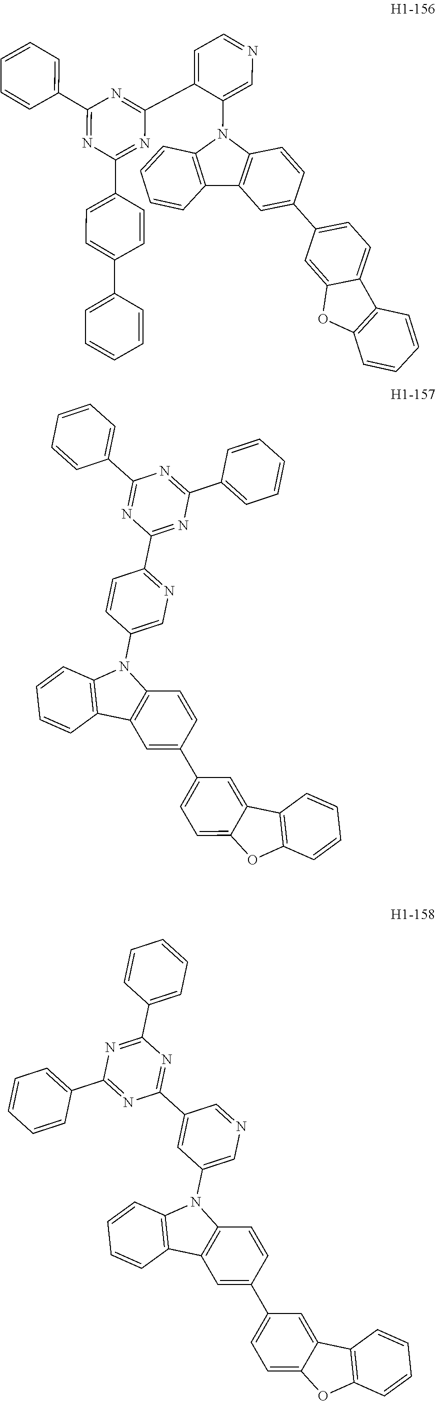 [SMILES: c1ccc(-c2ccc(-c3nc(-c4ccccc4)nc(-c4ccncc4-n4c5ccccc5c5cc(-c6ccc7c(c6)oc6ccccc67)ccc54)n3)cc2)cc1 ; c1ccc(-c2nc(-c3ccccc3)nc(-c3ccc(-n4c5ccccc5c5cc(-c6ccc7oc8ccccc8c7c6)ccc54)cn3)n2)cc1 ; c1ccc(-c2nc(-c3ccccc3)nc(-c3cncc(-n4c5ccccc5c5cc(-c6ccc7oc8ccccc8c7c6)ccc54)c3)n2)cc1]

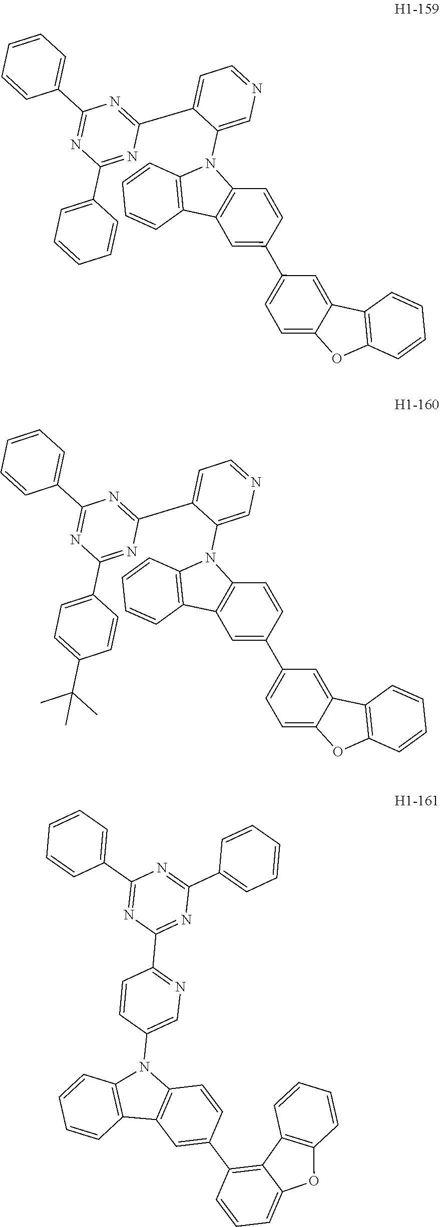 [SMILES: c1ccc(-c2nc(-c3ccccc3)nc(-c3ccncc3-n3c4ccccc4c4cc(-c5ccc6oc7ccccc7c6c5)ccc43)n2)cc1 ; CC(C)(C)c1ccc(-c2nc(-c3ccccc3)nc(-c3ccncc3-n3c4ccccc4c4cc(-c5ccc6oc7ccccc7c6c5)ccc43)n2)cc1 ; c1ccc(-c2nc(-c3ccccc3)nc(-c3ccc(-n4c5ccccc5c5cc(-c6cccc7oc8ccccc8c67)ccc54)cn3)n2)cc1]

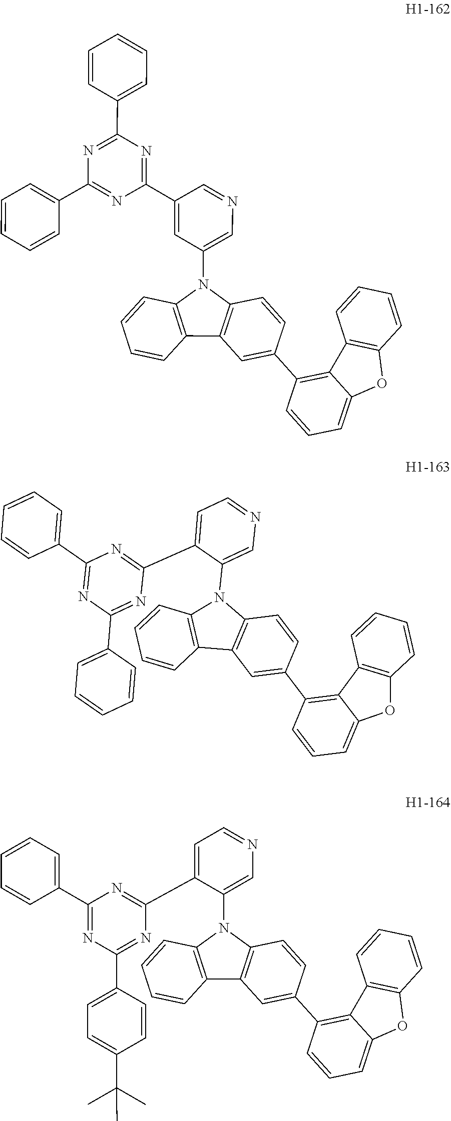 [SMILES: c1ccc(-c2nc(-c3ccccc3)nc(-c3cncc(-n4c5ccccc5c5cc(-c6cccc7oc8ccccc8c67)ccc54)c3)n2)cc1 ; c1ccc(-c2nc(-c3ccccc3)nc(-c3ccncc3-n3c4ccccc4c4cc(-c5cccc6oc7ccccc7c56)ccc43)n2)cc1 ; CC(C)(C)c1ccc(-c2nc(-c3ccccc3)nc(-c3ccncc3-n3c4ccccc4c4cc(-c5cccc6oc7ccccc7c56)ccc43)n2)cc1]

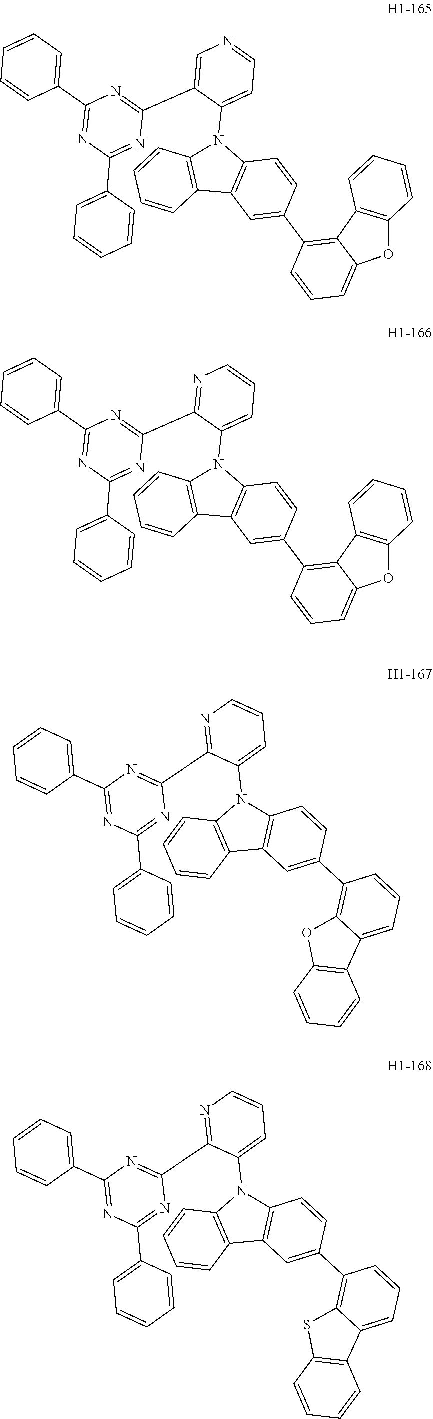 [SMILES: c1ccc(-c2nc(-c3ccccc3)nc(-c3cnccc3-n3c4ccccc4c4cc(-c5cccc6oc7ccccc7c56)ccc43)n2)cc1 ; c1ccc(-c2nc(-c3ccccc3)nc(-c3ncccc3-n3c4ccccc4c4cc(-c5cccc6oc7ccccc7c56)ccc43)n2)cc1 ; c1ccc(-c2nc(-c3ccccc3)nc(-c3ncccc3-n3c4ccccc4c4cc(-c5cccc6c5oc5ccccc56)ccc43)n2)cc1 ; c1ccc(-c2nc(-c3ccccc3)nc(-c3ncccc3-n3c4ccccc4c4cc(-c5cccc6c5sc5ccccc56)ccc43)n2)cc1]

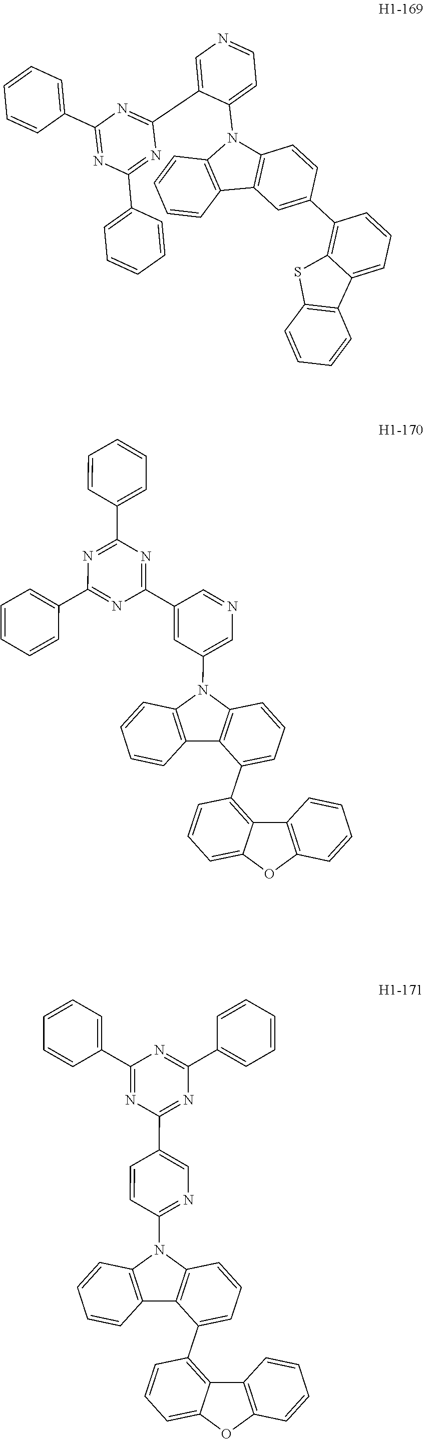 [SMILES: c1ccc(-c2nc(-c3ccccc3)nc(-c3cnccc3-n3c4ccccc4c4cc(-c5cccc6c5sc5ccccc56)ccc43)n2)cc1 ; c1ccc(-c2nc(-c3ccccc3)nc(-c3cncc(-n4c5ccccc5c5c(-c6cccc7oc8ccccc8c67)cccc54)c3)n2)cc1 ; c1ccc(-c2nc(-c3ccccc3)nc(-c3ccc(-n4c5ccccc5c5c(-c6cccc7oc8ccccc8c67)cccc54)nc3)n2)cc1]

The organic electroluminescent compound represented by formula 1 according to the present disclosure may be prepared by a synthetic method known to one skilled in the art, and for example may be prepared as shown in the following reaction schemes 1 and 2, but is not limited thereto.

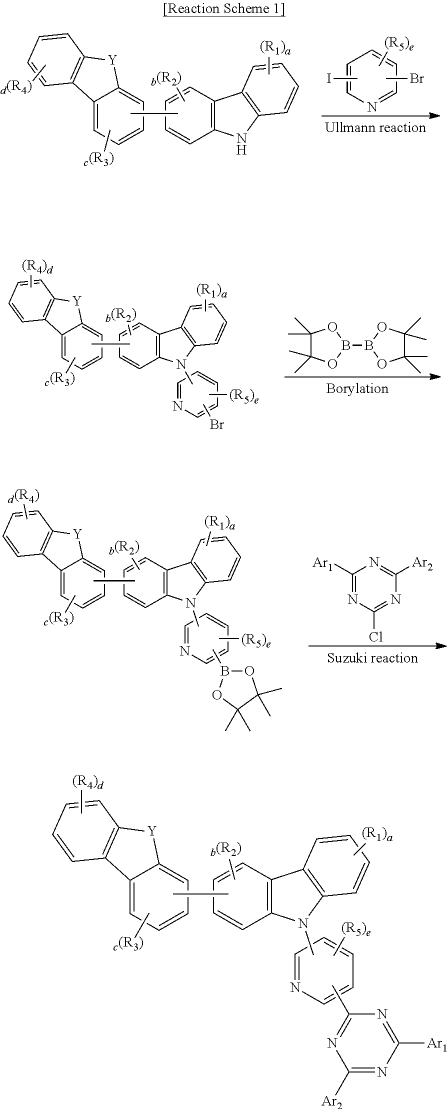

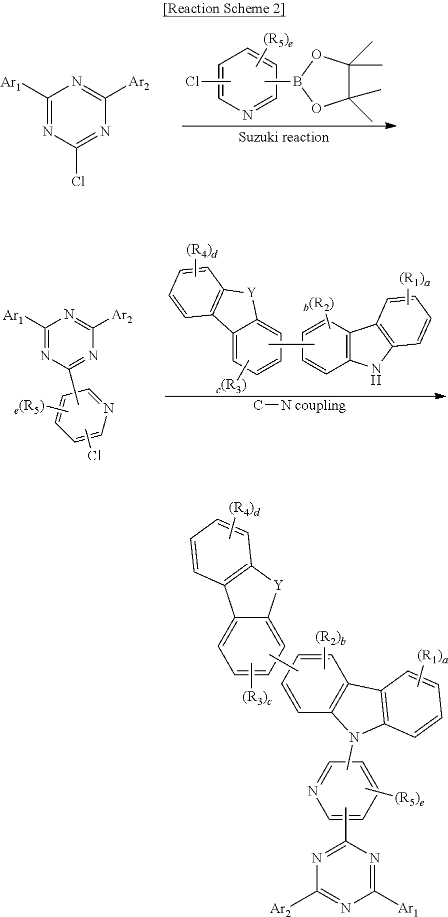

In reaction schemes 1 and 2,Ar₁, Ar₂, Y, R₁ to R₅, and a to d are as defined in formula 1.

The present disclosure may provide a plurality of host materials comprising a first host material comprising the organic electroluminescent compound represented by formula 1 and a second host material. For example, the plurality of host materials of the present disclosure may comprise at least one compound represented by formula 1 and at least one compound represented by the following formula 11.

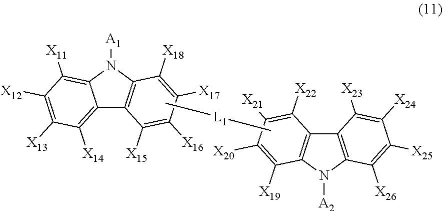

In formula 11, A₁ and A₂, each independently, represent a substituted or unsubstituted (C6-C30)aryl, or a substituted or unsubstituted (3- to 30-membered)heteroaryl; L₁ represents a single bond, or a substituted or unsubstituted (C6-C30)arylene; and X₁₁ to X₂₆, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted (3- to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di- (C1-C30)alkylamino, or a substituted or unsubstituted mono- or di- (C6-C30)arylamino; or at least two adjacent ones of X₁₁ to X₂₆ may be linked to each other to form a ring(s).

In formula 11, according to one embodiment of the present disclosure, A₁ and A₂, each independently, represent a substituted or unsubstituted (C6-C25)aryl, or a substituted or unsubstituted (5- to 25-membered)heteroaryl. According to another embodiment of the present disclosure, A₁ and A₂, each independently, represent a (C6-C18)aryl unsubstituted or substituted with at least one of a (C1-C4)alkyl(s), a (6-to 18-membered)heteroaryl(s), and a tri(C6-C18)arylsilyl(s), or an unsubstituted (5-to 20-membered)heteroaryl. For example, A₁ and A₂, each independently, may be a phenyl unsubstituted or substituted with a methyl(s), a triphenylsilyl(s), a pyridyl(s), or a phenylpyridyl(s); a naphthyl; a biphenyl; a naphthylphenyl; a dimethylfluorenyl; a diphenylfluorenyl; a dimethylbenzofluorenyl; a pyridyl; a pyrimidyl; a carbazolyl; a dibenzofuranyl; or a dibenzothiophenyl, etc.

In formula 11, according to one embodiment of the present disclosure, L₁ represents a single bond, or an unsubstituted (C6-C25)arylene. According to another embodiment of the present disclosure, L₁ represents a single bond, or an unsubstituted (C6-C18)arylene. For example, L₁ may be a single bond, a phenylene, a naphthylene, or a biphenylene, etc.

In formula 11, according to one embodiment of the present disclosure, X₁₁ to X₂₆, each independently, represent hydrogen, deuterium, a substituted or unsubstituted (C1-C25)alkyl, a substituted or unsubstituted (C6-C30)aryl, or a substituted or unsubstituted (5- to 25-membered)heteroaryl; or at least two adjacent ones of X₁₁ to X₂₆ may be linked to each other to form a substituted or unsubstituted mono- or polycyclic (6- to 30-membered) alicyclic or aromatic ring. According to another embodiment of the present disclosure, X₁₁ to X₂₆, each independently, represent hydrogen, or an unsubstituted (5- to 20-membered)heteroaryl; or two adjacent ones of X₁₁ to X₂₆ may be linked to each other to form an unsubstituted monocyclic (6- to 10-membered) aromatic ring. For example, X₁₁ to X₂₆, each independently, may be hydrogen, a dibenzothiophene, or a dibenzofuran, etc; or two adjacent ones of X₁₁ to X₂₆ may be linked to each other to form a benzene ring.

The compound represented by formula 11 may be selected from the following compounds, but is not limited thereto.

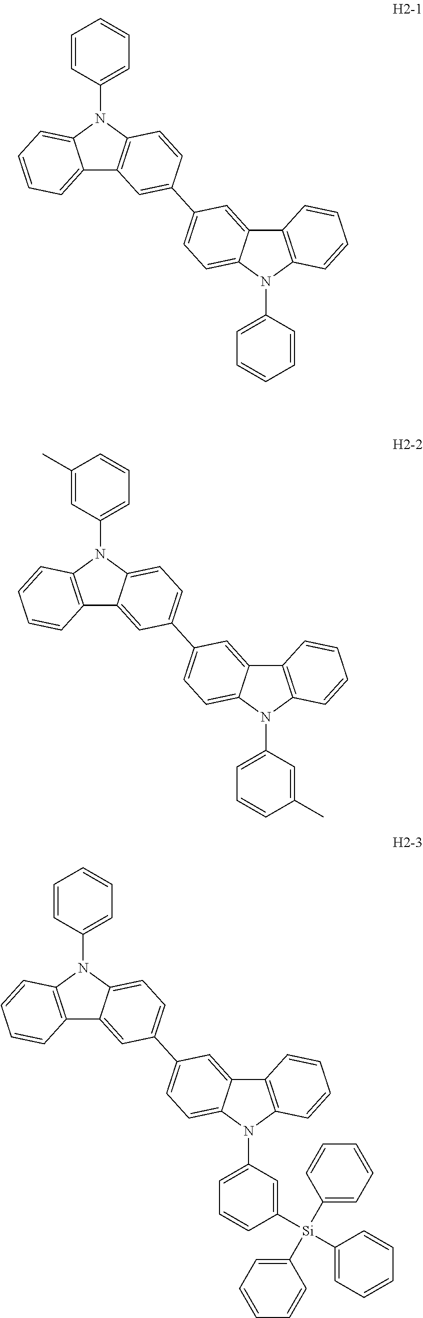 [SMILES: c1ccc(-n2c3ccccc3c3cc(-c4ccc5c(c4)c4ccccc4n5-c4ccccc4)ccc32)cc1 ; Cc1cccc(-n2c3ccccc3c3cc(-c4ccc5c(c4)c4ccccc4n5-c4cccc(C)c4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

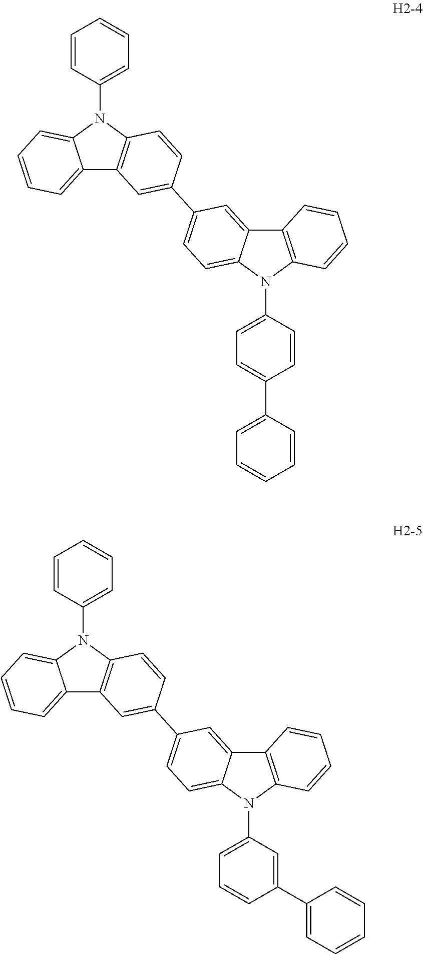 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1]

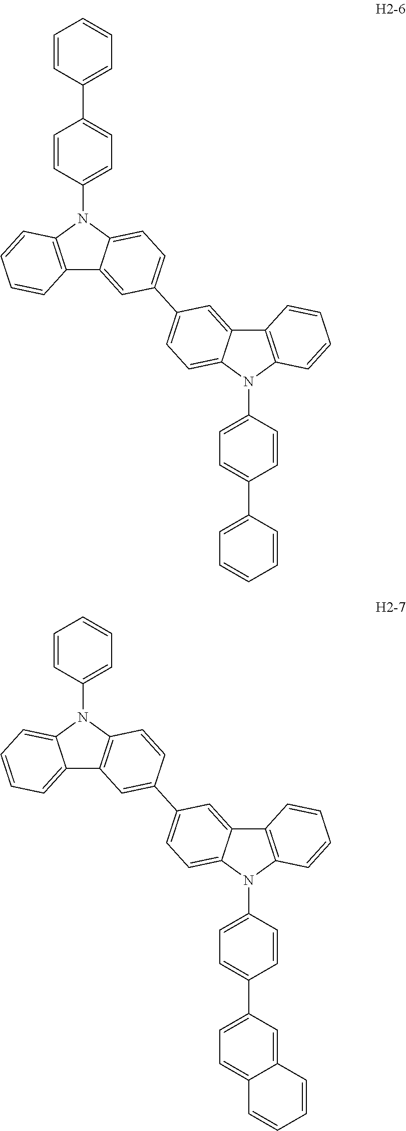 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-n2c3ccccc3c3cc(-c4ccc5c(c4)c4ccccc4n5-c4ccc(-c5ccc6ccccc6c5)cc4)ccc32)cc1]

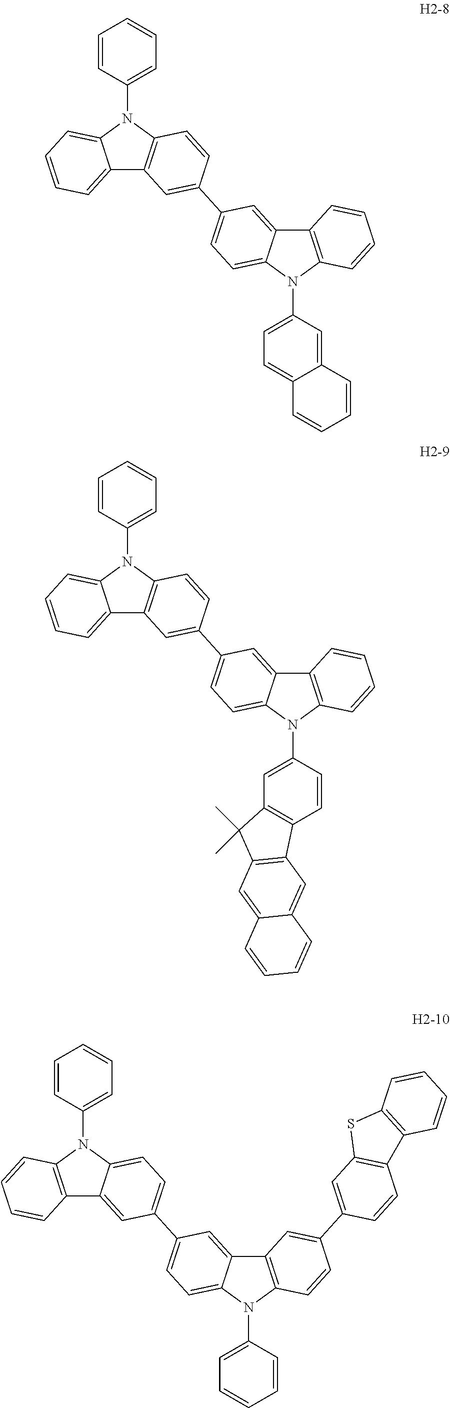 [SMILES: c1ccc(-n2c3ccccc3c3cc(-c4ccc5c(c4)c4ccccc4n5-c4ccc5ccccc5c4)ccc32)cc1 ; CC1(C)c2cc(-n3c4ccccc4c4cc(-c5ccc6c(c5)c5ccccc5n6-c5ccccc5)ccc43)ccc2-c2cc3ccccc3cc21 ; c1ccc(-n2c3ccccc3c3cc(-c4ccc5c(c4)c4cc(-c6ccc7c(c6)sc6ccccc67)ccc4n5-c4ccccc4)ccc32)cc1]

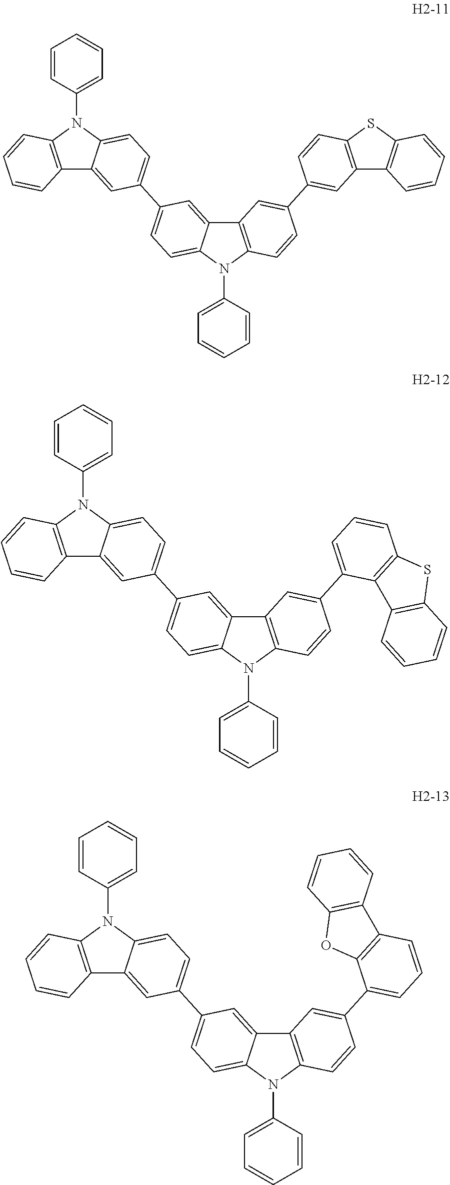 [SMILES: c1ccc(-n2c3ccccc3c3cc(-c4ccc5c(c4)c4cc(-c6ccc7sc8ccccc8c7c6)ccc4n5-c4ccccc4)ccc32)cc1 ; c1ccc(-n2c3ccccc3c3cc(-c4ccc5c(c4)c4cc(-c6cccc7sc8ccccc8c67)ccc4n5-c4ccccc4)ccc32)cc1 ; c1ccc(-n2c3ccccc3c3cc(-c4ccc5c(c4)c4cc(-c6cccc7c6oc6ccccc67)ccc4n5-c4ccccc4)ccc32)cc1]

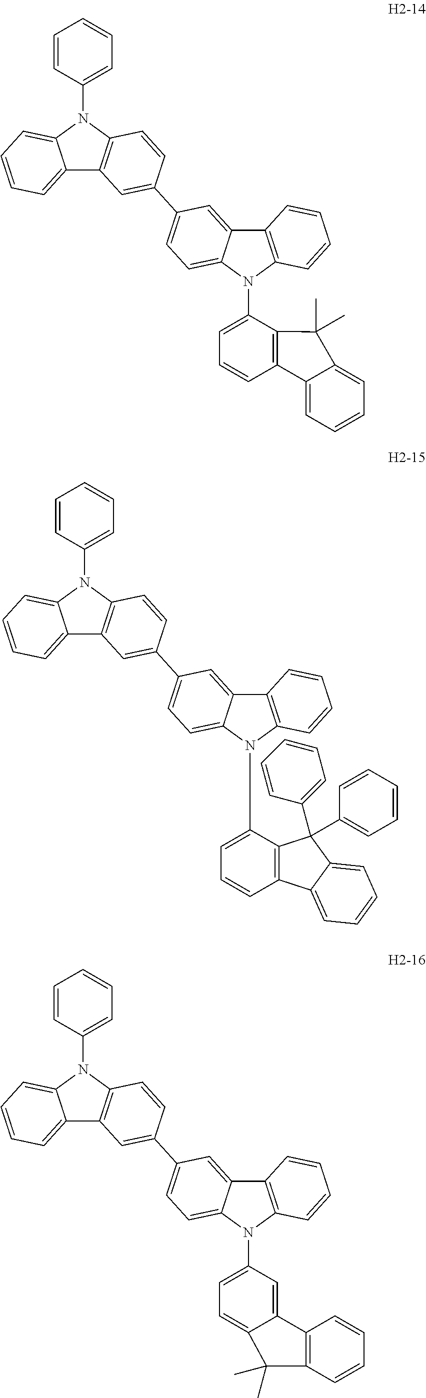 [SMILES: CC1(C)c2ccccc2-c2cccc(-n3c4ccccc4c4cc(-c5ccc6c(c5)c5ccccc5n6-c5ccccc5)ccc43)c21 ; c1ccc(-n2c3ccccc3c3cc(-c4ccc5c(c4)c4ccccc4n5-c4cccc5c4C(c4ccccc4)(c4ccccc4)c4ccccc4-5)ccc32)cc1 ; CC1(C)c2ccccc2-c2cc(-n3c4ccccc4c4cc(-c5ccc6c(c5)c5ccccc5n6-c5ccccc5)ccc43)ccc21]

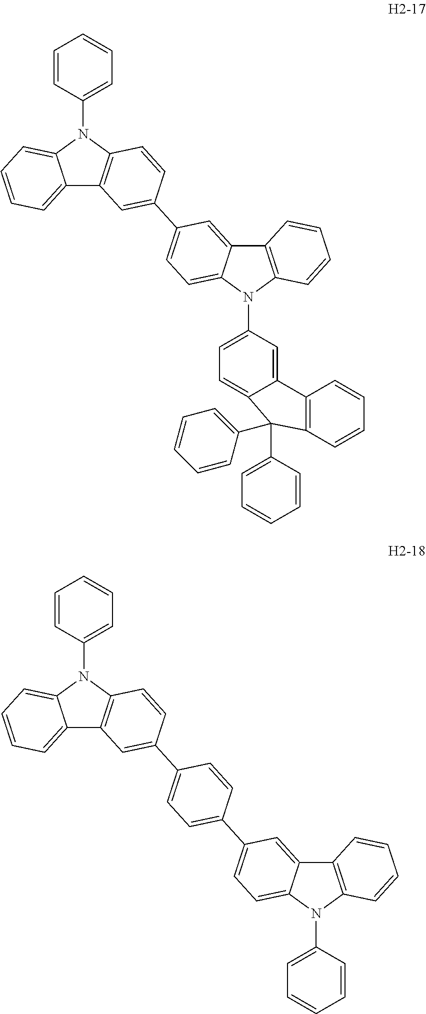 [SMILES: c1ccc(-n2c3ccccc3c3cc(-c4ccc5c(c4)c4ccccc4n5-c4ccc5c(c4)-c4ccccc4C5(c4ccccc4)c4ccccc4)ccc32)cc1 ; c1ccc(-n2c3ccccc3c3cc(-c4ccc(-c5ccc6c(c5)c5ccccc5n6-c5ccccc5)cc4)ccc32)cc1]

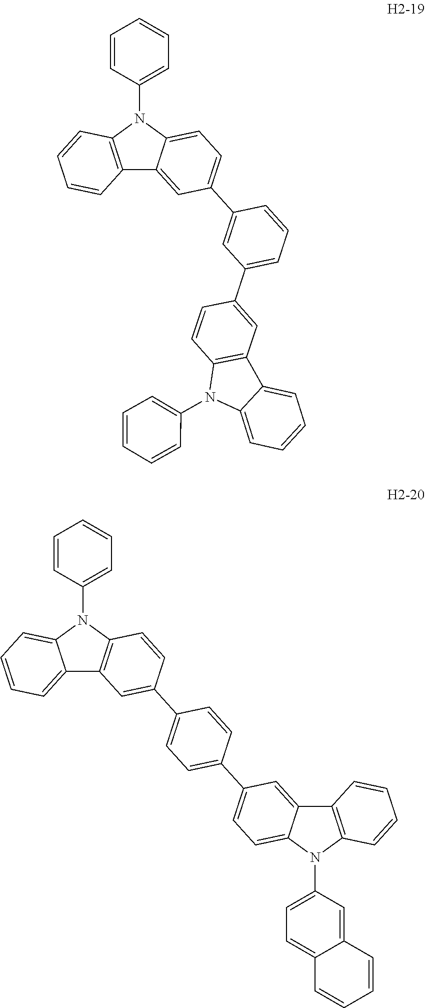 [SMILES: c1ccc(-n2c3ccccc3c3cc(-c4cccc(-c5ccc6c(c5)c5ccccc5n6-c5ccccc5)c4)ccc32)cc1 ; c1ccc(-n2c3ccccc3c3cc(-c4ccc(-c5ccc6c(c5)c5ccccc5n6-c5ccc6ccccc6c5)cc4)ccc32)cc1]

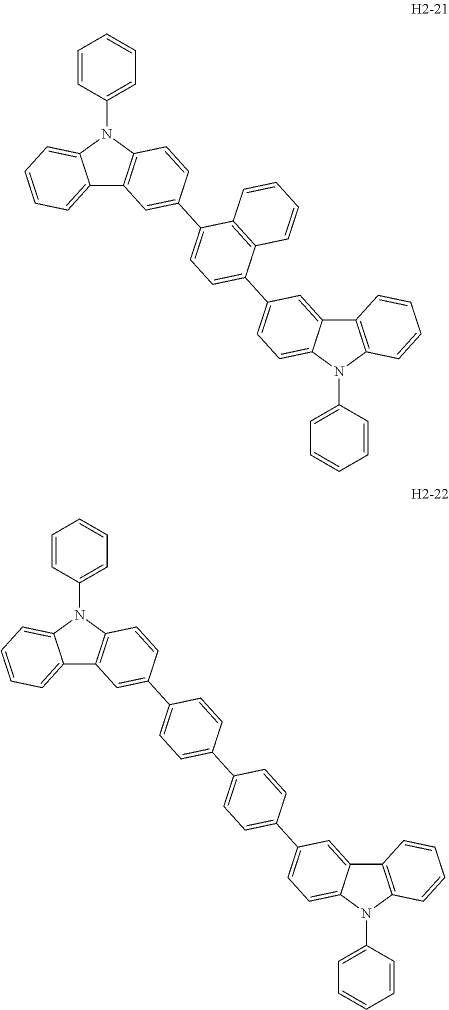 [SMILES: c1ccc(-n2c3ccccc3c3cc(-c4ccc(-c5ccc6c(c5)c5ccccc5n6-c5ccccc5)c5ccccc45)ccc32)cc1 ; c1ccc(-n2c3ccccc3c3cc(-c4ccc(-c5ccc(-c6ccc7c(c6)c6ccccc6n7-c6ccccc6)cc5)cc4)ccc32)cc1]

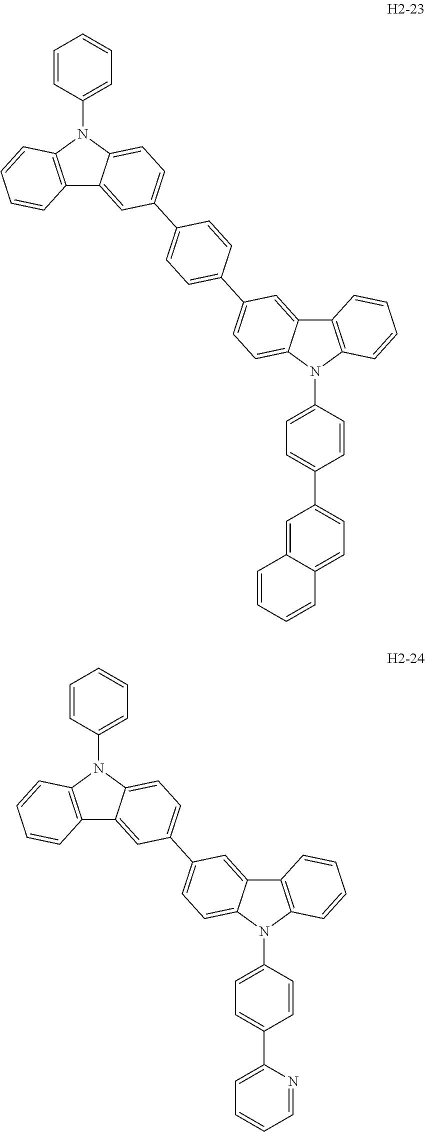 [SMILES: c1ccc(-n2c3ccccc3c3cc(-c4ccc(-c5ccc6c(c5)c5ccccc5n6-c5ccc(-c6ccc7ccccc7c6)cc5)cc4)ccc32)cc1 ; c1ccc(-n2c3ccccc3c3cc(-c4ccc5c(c4)c4ccccc4n5-c4ccc(-c5ccccn5)cc4)ccc32)cc1]

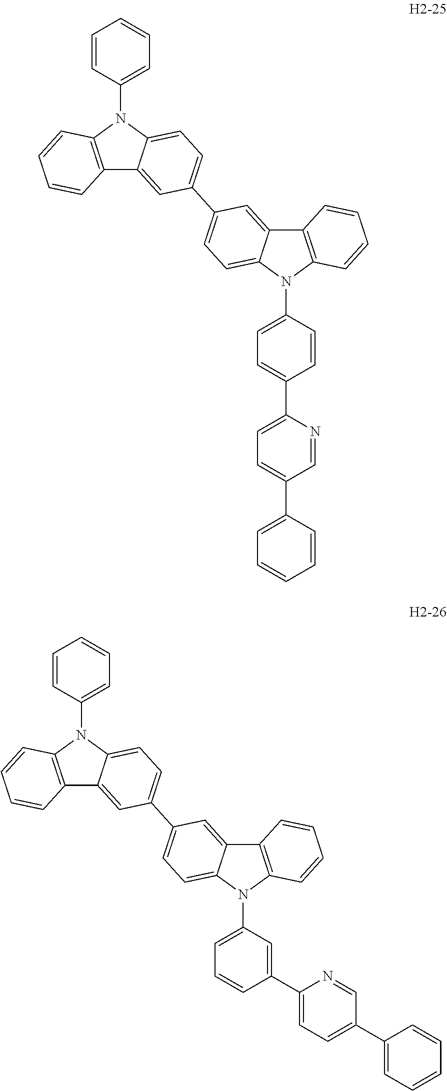 [SMILES: c1ccc(-c2ccc(-c3ccc(-n4c5ccccc5c5cc(-c6ccc7c(c6)c6ccccc6n7-c6ccccc6)ccc54)cc3)nc2)cc1 ; c1ccc(-c2ccc(-c3cccc(-n4c5ccccc5c5cc(-c6ccc7c(c6)c6ccccc6n7-c6ccccc6)ccc54)c3)nc2)cc1]

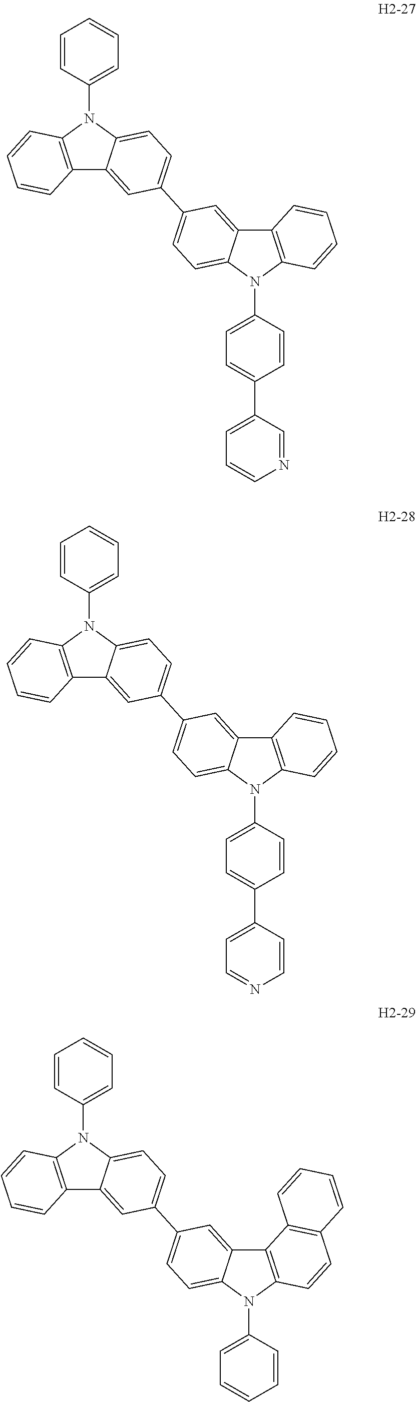 [SMILES: c1ccc(-n2c3ccccc3c3cc(-c4ccc5c(c4)c4ccccc4n5-c4ccc(-c5cccnc5)cc4)ccc32)cc1 ; c1ccc(-n2c3ccccc3c3cc(-c4ccc5c(c4)c4ccccc4n5-c4ccc(-c5ccncc5)cc4)ccc32)cc1 ; c1ccc(-n2c3ccccc3c3cc(-c4ccc5c(c4)c4c6ccccc6ccc4n5-c4ccccc4)ccc32)cc1]

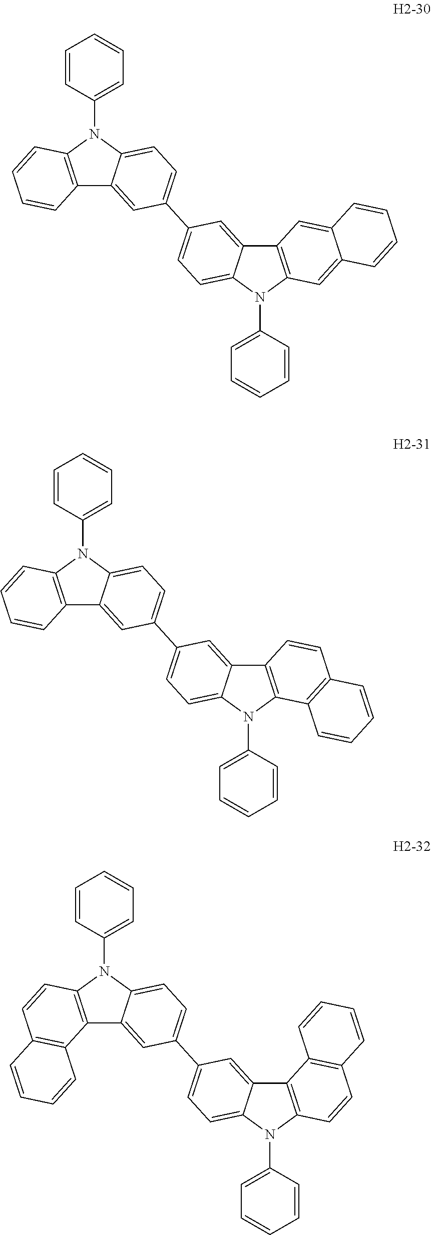 [SMILES: c1ccc(-n2c3ccccc3c3cc(-c4ccc5c(c4)c4cc6ccccc6cc4n5-c4ccccc4)ccc32)cc1 ; c1ccc(-n2c3ccccc3c3cc(-c4ccc5c(c4)c4ccc6ccccc6c4n5-c4ccccc4)ccc32)cc1 ; c1ccc(-n2c3ccc(-c4ccc5c(c4)c4c6ccccc6ccc4n5-c4ccccc4)cc3c3c4ccccc4ccc32)cc1]

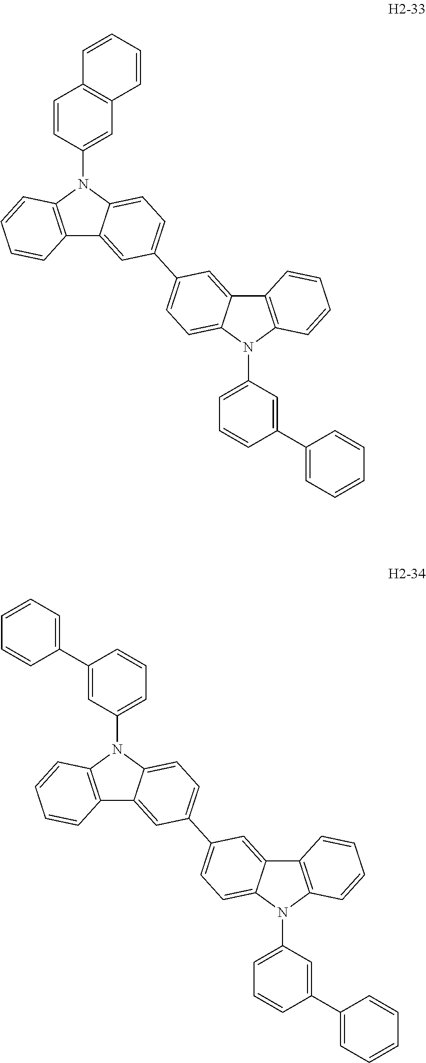 [SMILES: c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5cccc(-c6ccccc6)c5)ccc43)c2)cc1]

The organic electroluminescent compound represented by formula 11 according to the present disclosure may be prepared by a synthetic method known to one skilled in the art, and for example may be prepared by referring to the method disclosed in Japanese Patent Publication No. JP 3139321 B (published on Feb. 26, 2001) and International Publication No. WO 2011/162162 (published on Dec. 29, 2011), but is not limited thereto.

The first and second host materials of the present disclosure may be comprised together in one light-emitting layer or may respectively be comprised in different light-emitting layers. The composite material for an organic electroluminescent device of the present disclosure may comprise the compound represented by formula 1 and the compound represented by formula 11 in a ratio of about 1:99 to about 99:1, preferably in a ratio of about 10:90 to about 90:10, and more preferably in a ratio of about 30:70 to about 70:30. Also, the compound represented by formula 1 and the compound represented by formula 11 may be combined by mixing them in a shaker, by dissolving them in a glass tube by heat, or by dissolving them in a solvent, etc.

The present disclosure may provide an organic electroluminescent device comprising the compound represented by formula 1 or a plurality of host materials according to one embodiment of the present disclosure. Specifically, the organic electroluminescent device may comprise the compound represented by formula 1, and may further comprise at least one other organic electroluminescent compound. For example, the organic electroluminescent device may comprise at least one compound represented by formula 1 and at least one compound represented by formula 11.

In addition, the present disclosure may provide an organic electroluminescent material comprising the organic electroluminescent compound of formula 1, and an organic electroluminescent device comprising the organic electroluminescent material. The organic electroluminescent material may consist of the organic electroluminescent compound of the present disclosure as a sole compound, or may further comprise conventional materials generally used in organic electroluminescent materials.

Meanwhile, the organic electroluminescent device of the present disclosure may comprise a first electrode, a second electrode, and at least one organic layer between the first and second electrodes. The organic layer may comprise at least one organic electroluminescent compound of formula 1. The organic layer may further comprise at least one compound selected from the group consisting of arylamine-based compounds and styrylarylamine-based compounds. Also, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising the metal.

One of the first and second electrodes may be an anode, and the other may be a cathode. The organic layer may comprise a light-emitting layer, and may further comprise at least one layer selected from a hole injection layer, a hole transport layer, a hole auxiliary layer, a light-emitting auxiliary layer, an electron transport layer, an electron injection layer, an interlayer, a hole blocking layer, an electron blocking layer, and an electron buffer layer.

The present disclosure may comprise a hole transport zone between an anode and a light-emitting layer, and the hole transport zone may comprise at least one of a hole injection layer, a hole transport layer, a hole auxiliary layer, a light-emitting auxiliary layer and an electron blocking layer. The hole injection layer, the hole transport layer, the hole auxiliary layer, the light-emitting auxiliary layer and the electron blocking layer, respectively, may be a single layer or a plurality of layers in which two or more layers are stacked. The hole injection layer may be multi-layers in order to lower the hole injection barrier (or hole injection voltage) from the anode to the hole transport layer or the electron blocking layer, wherein two compounds may be used simultaneously in each of the multi-layers. The electron blocking layer may be placed between the hole transport layer (or the hole injection layer) and the light-emitting layer, and can confine the excitons within the light-emitting layer by blocking the overflow of electrons from the light-emitting layer to prevent a light-emitting leakage.

The dopant comprised in the organic electroluminescent device of the present disclosure may be at least one phosphorescent or fluorescent dopant, and is preferably at least one phosphorescent dopant. The phosphorescent dopant material applied to the organic electroluminescent device of the present disclosure is not particularly limited, but may be preferably selected from the metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

The dopant comprised in the organic electroluminescent device of the present disclosure may comprise a compound represented by the following formula 101, but is not limited thereto.

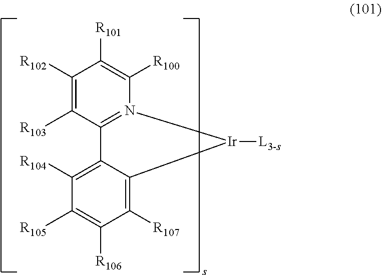

In formula 101, L is selected from the following structures 1 and 2:

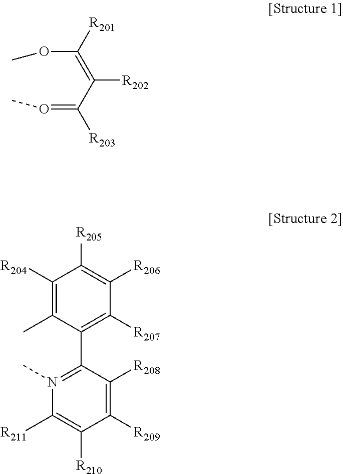

R₁₀₀ to R₁₀₃, each independently, represent hydrogen, deuterium, a halogen, a (C1-C30)alkyl unsubstituted or substituted with a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, benzofuropyridine, benzothienopyridine, indenopyridine, benzofuroquinoline, benzothienoquinoline, or indenoquinoline ring, together with pyridine;

R₁₀₄ to R₁₀₇, each independently, represent hydrogen, deuterium, a halogen, a (C1-C30)alkyl unsubstituted or substituted with a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a ring(s), e.g., a substituted or unsubstituted, naphthalene, fluorene, dibenzothiophene, dibenzofuran, indenopyridine, benzofuropyridine, or benzothienopyridine ring, together with benzene;

R₂₀₁ to R₂₁₁, each independently, represent hydrogen, deuterium, a halogen, a (C1-C30)alkyl unsubstituted or substituted with a halogen(s), a substituted or unsubstituted (C3-C30)cycloalkyl, or a substituted or unsubstituted (C6-C30)aryl; or may be linked to an adjacent substituent to form a ring(s); and

s represents an integer of 1 to 3.

The specific examples of the dopant compound are as follows, but are not limited thereto.

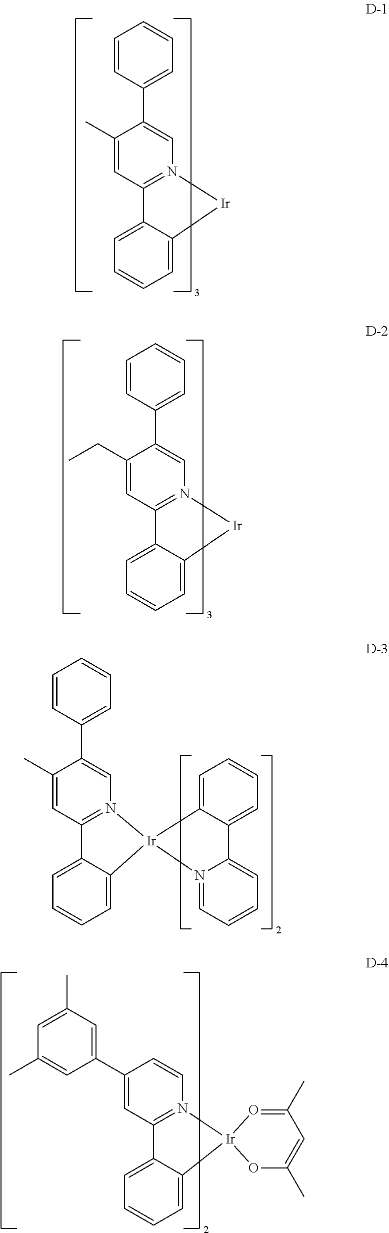

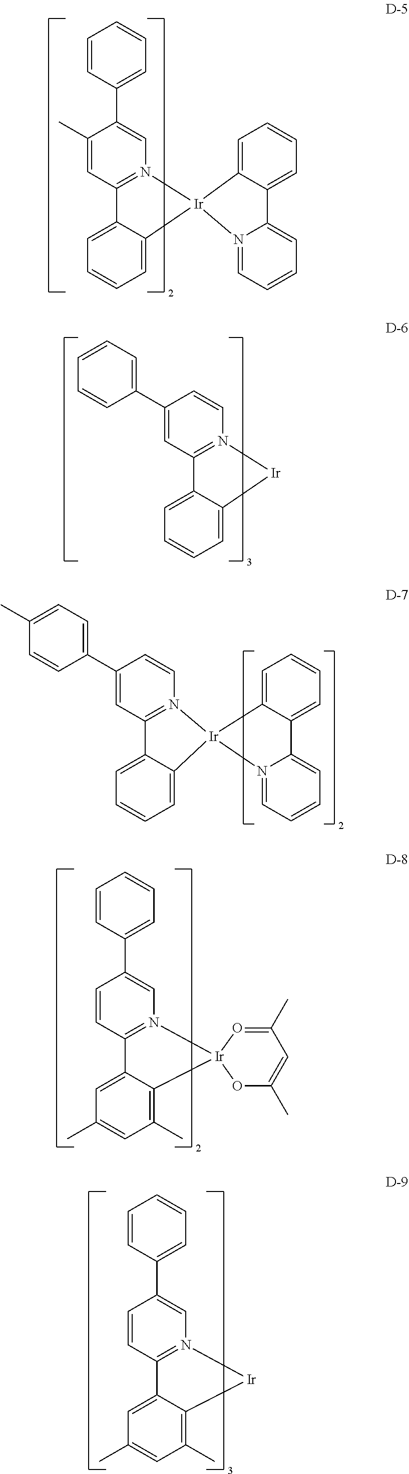

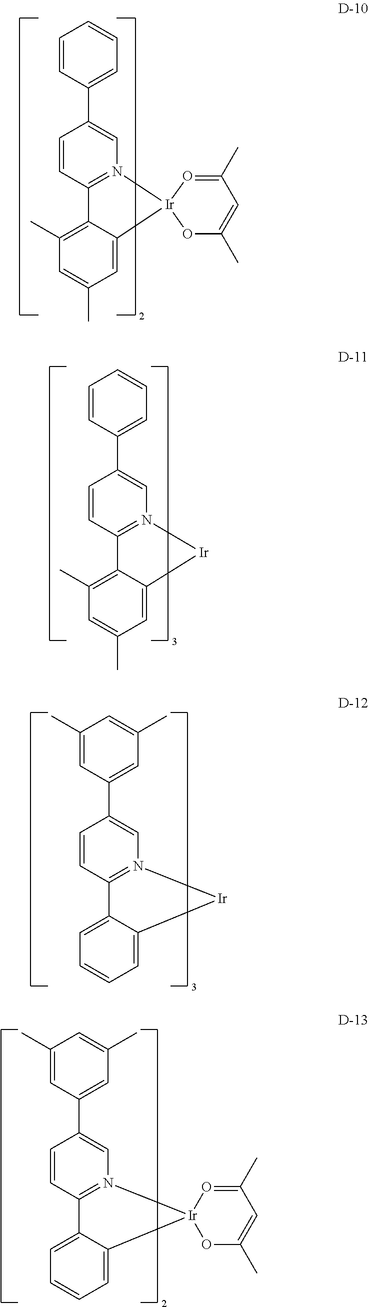

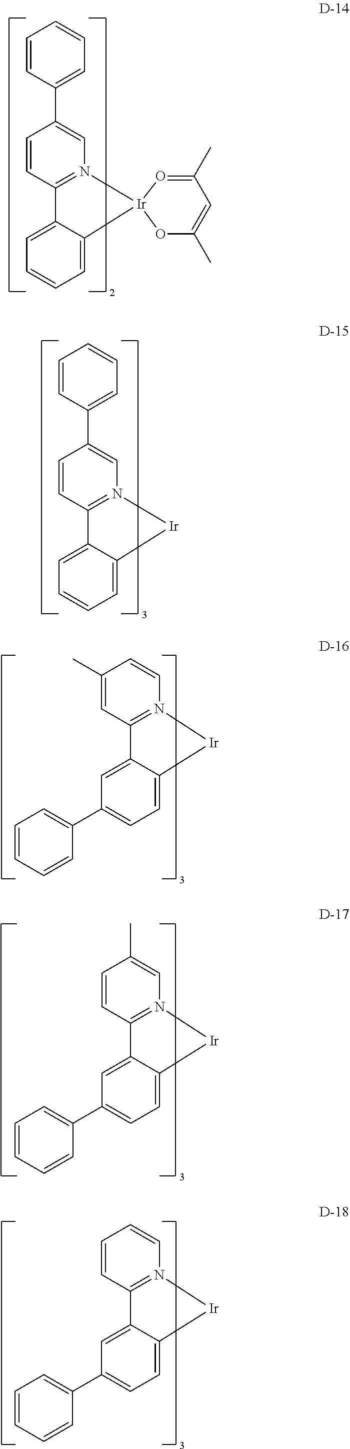

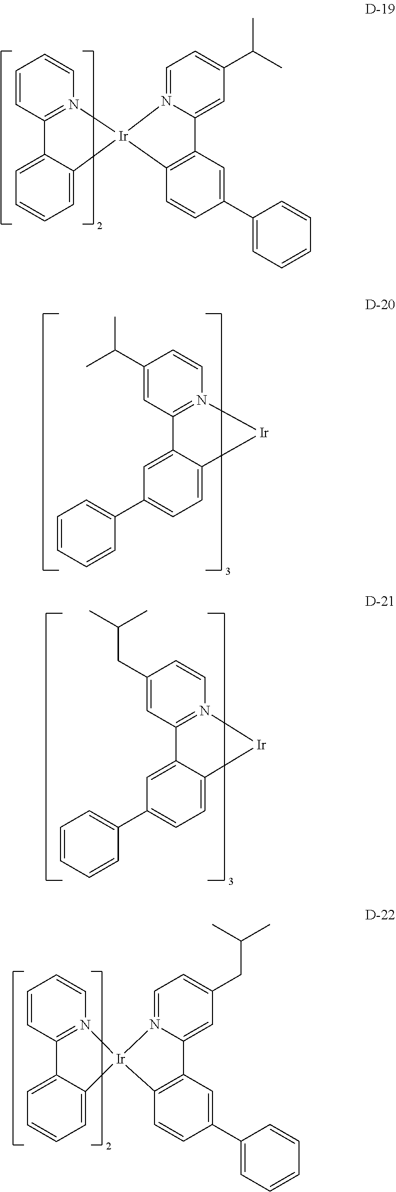

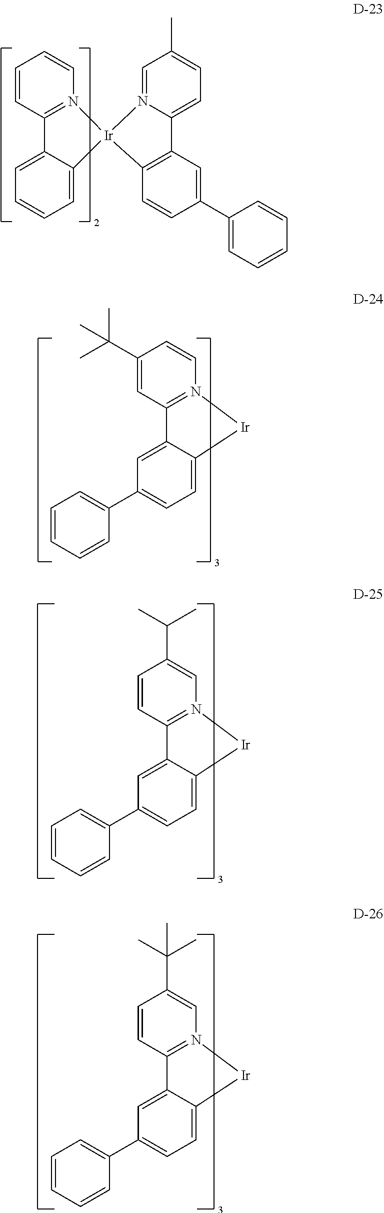

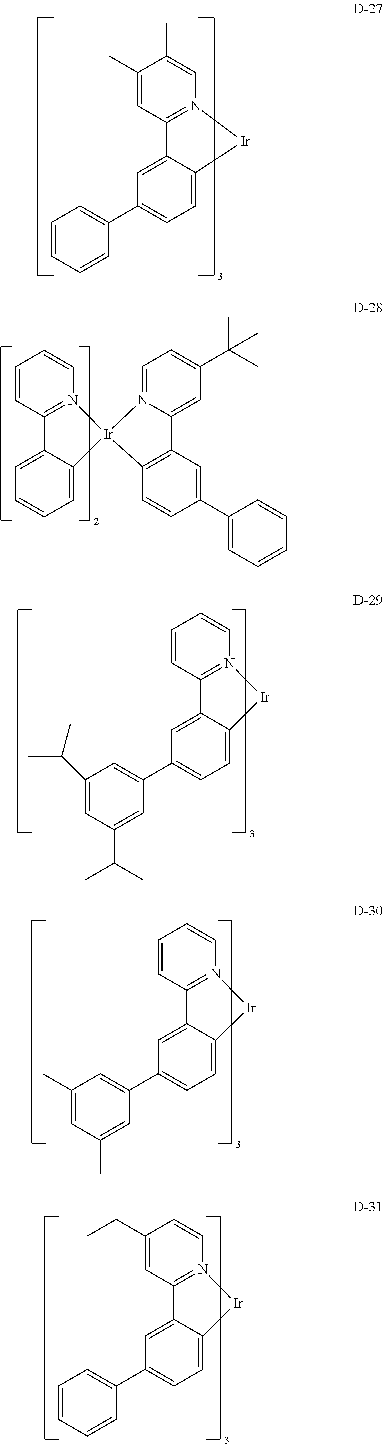

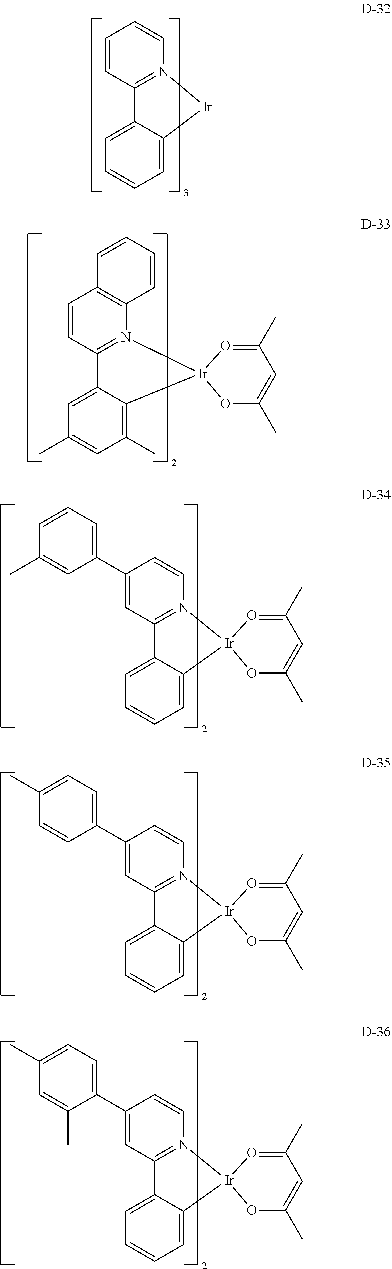

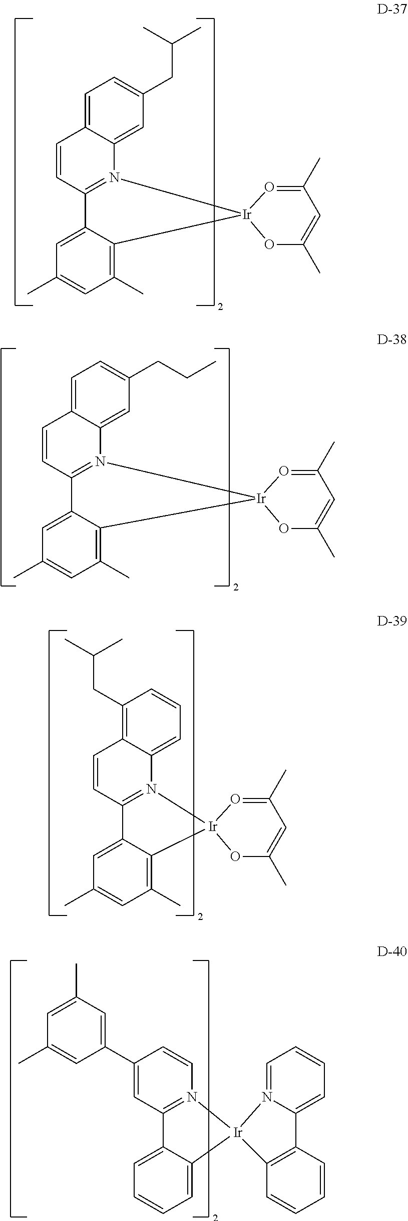

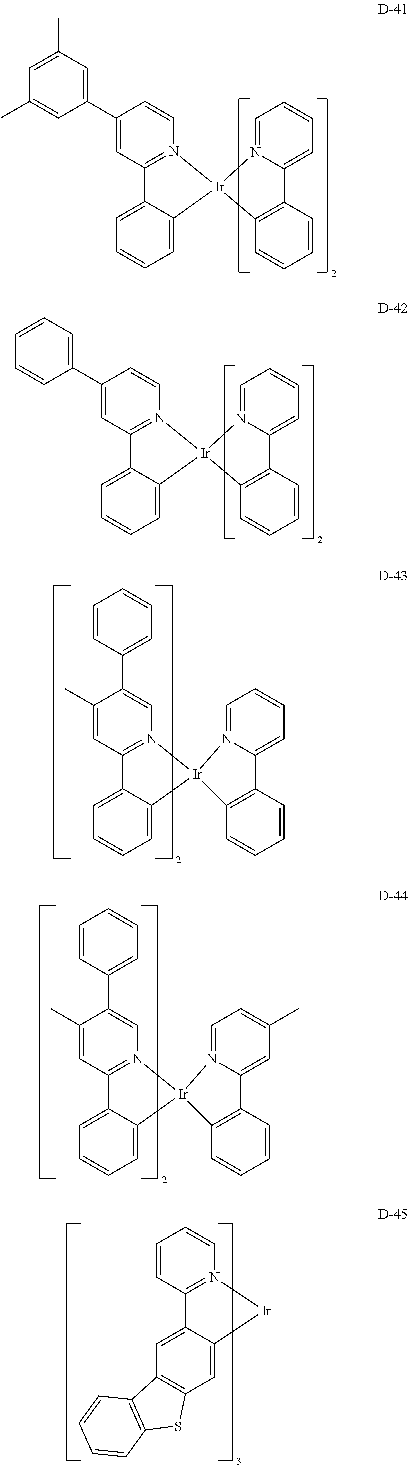

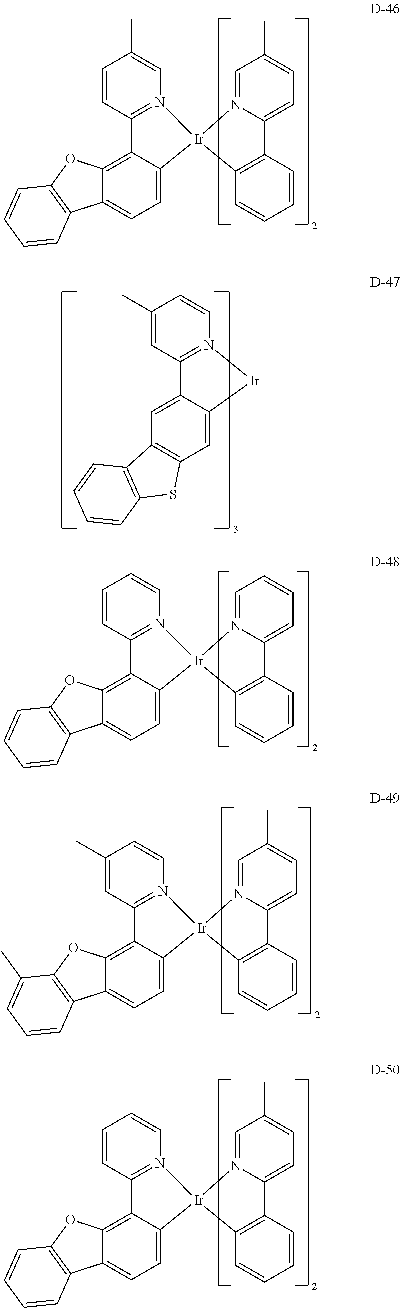

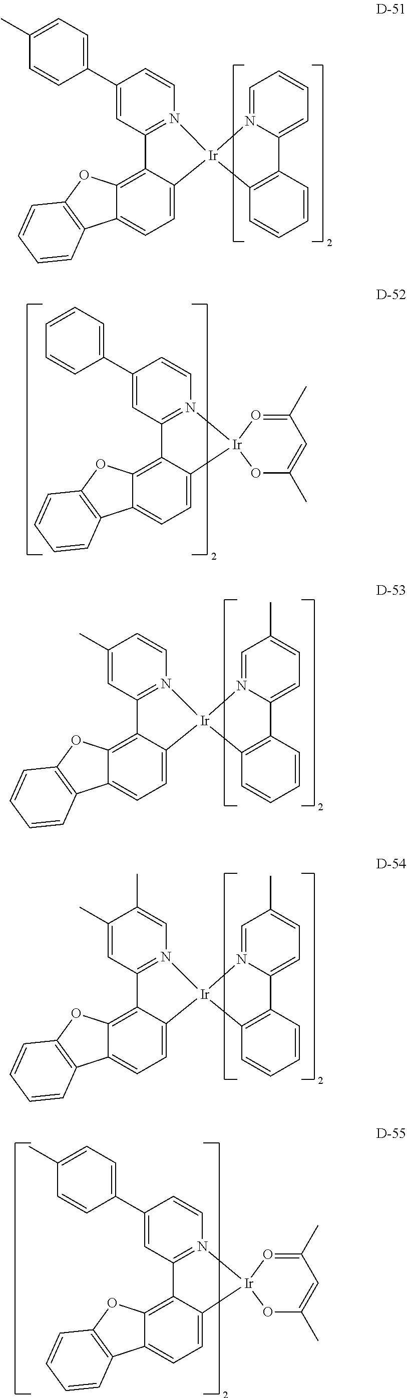

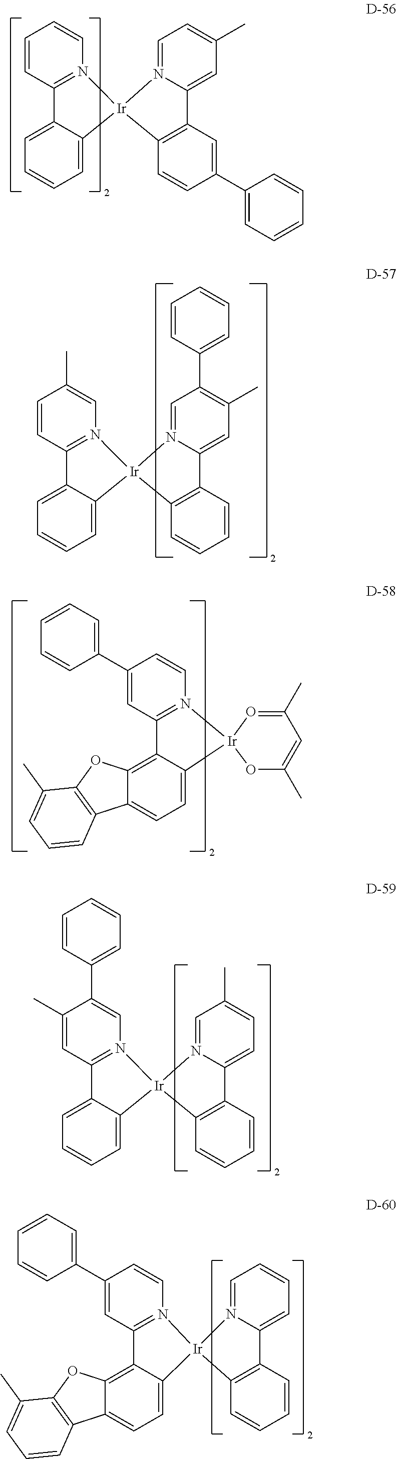

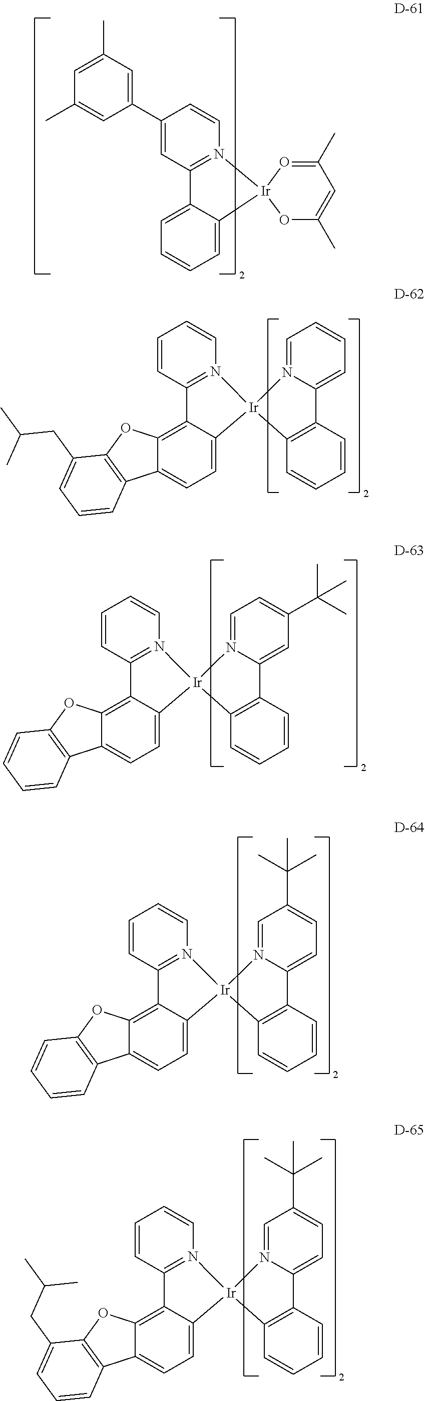

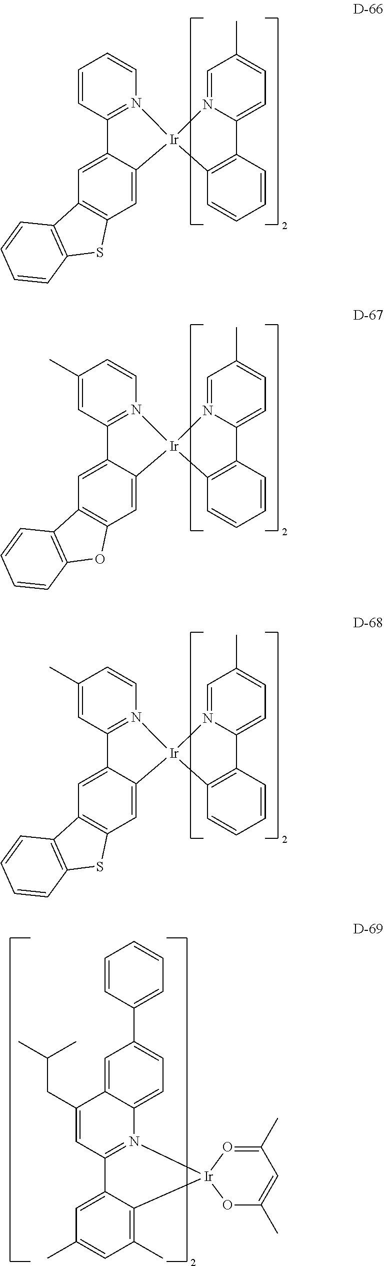

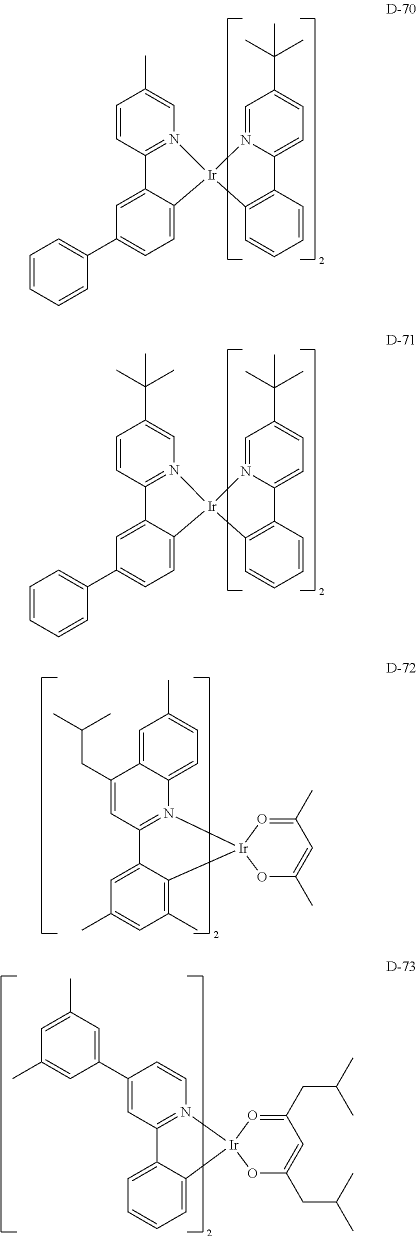

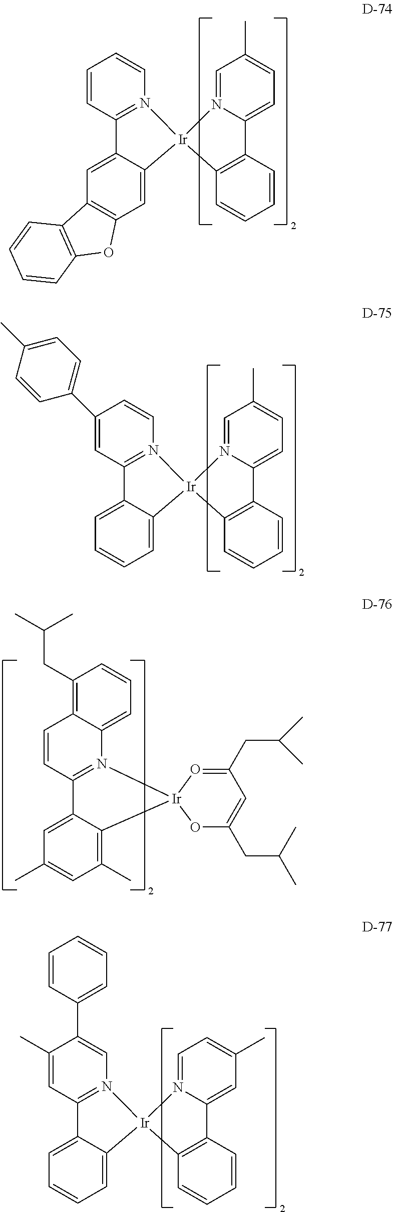

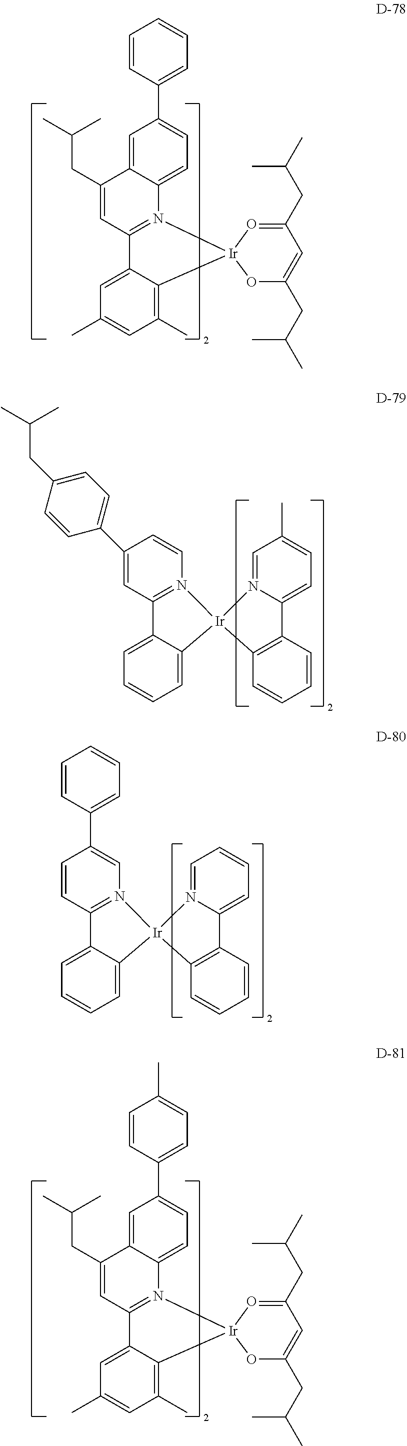

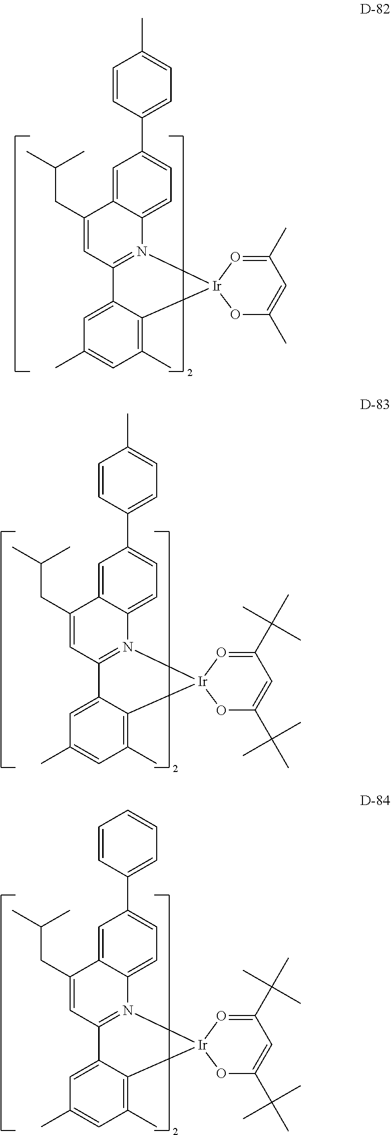

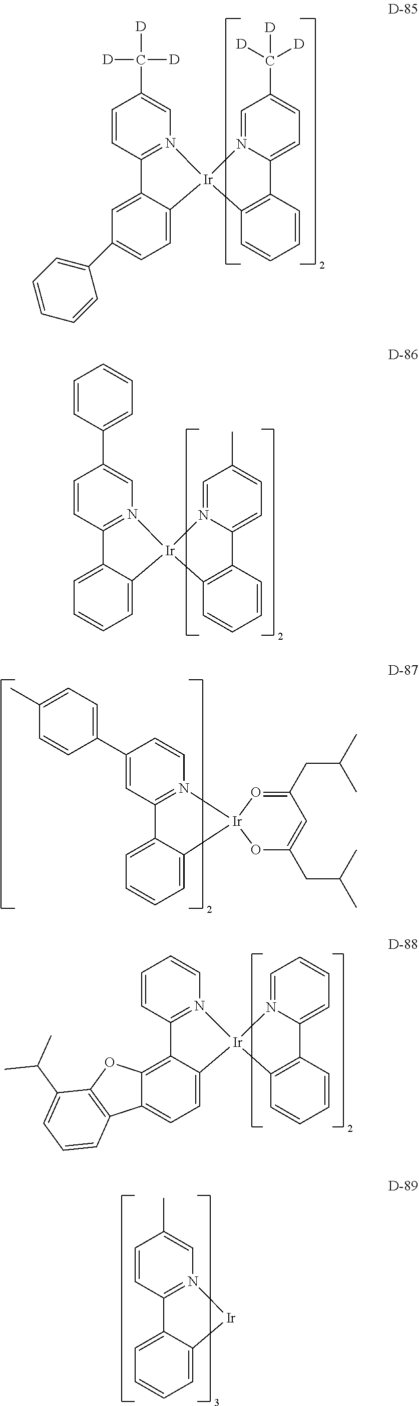

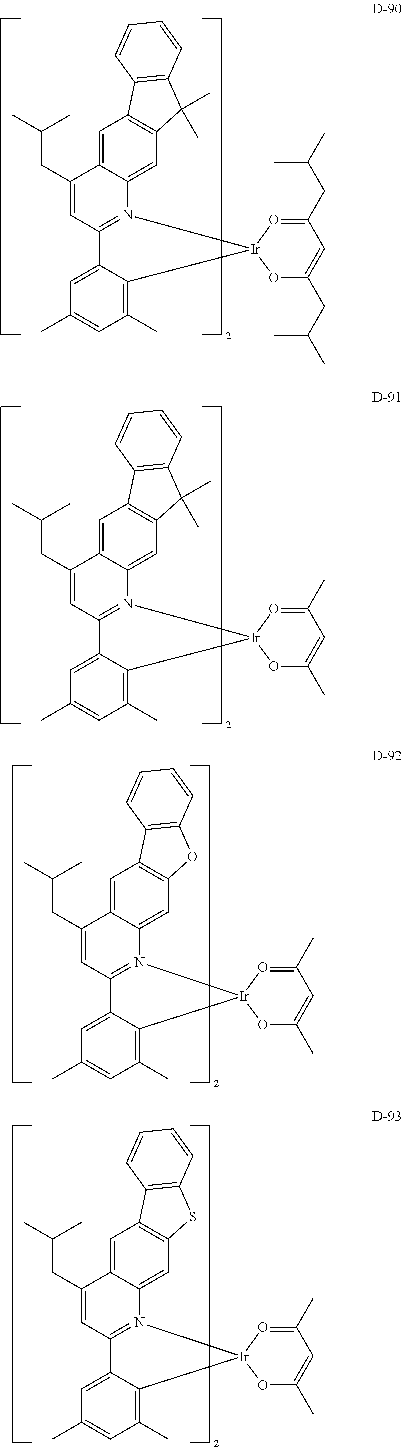

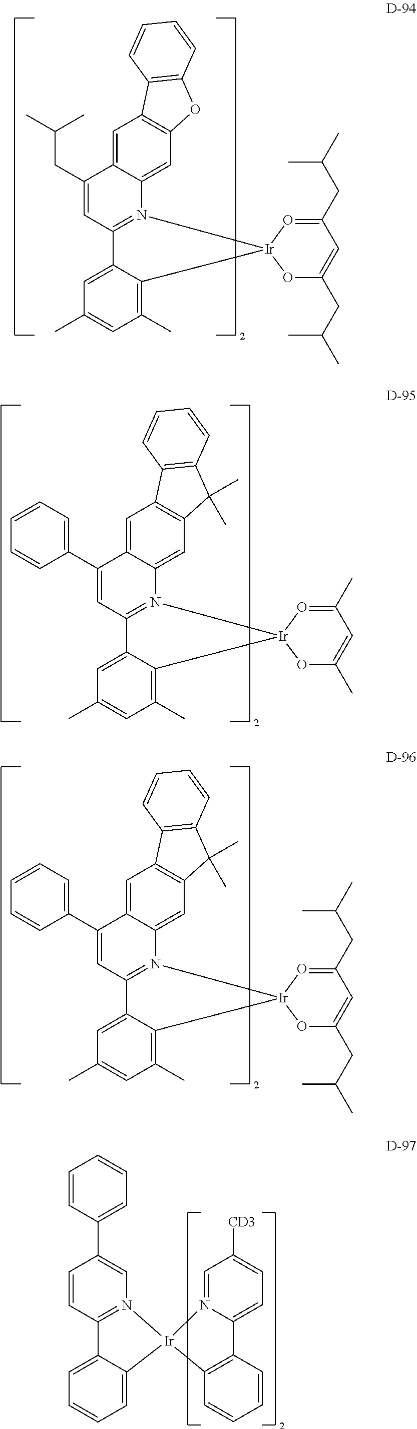

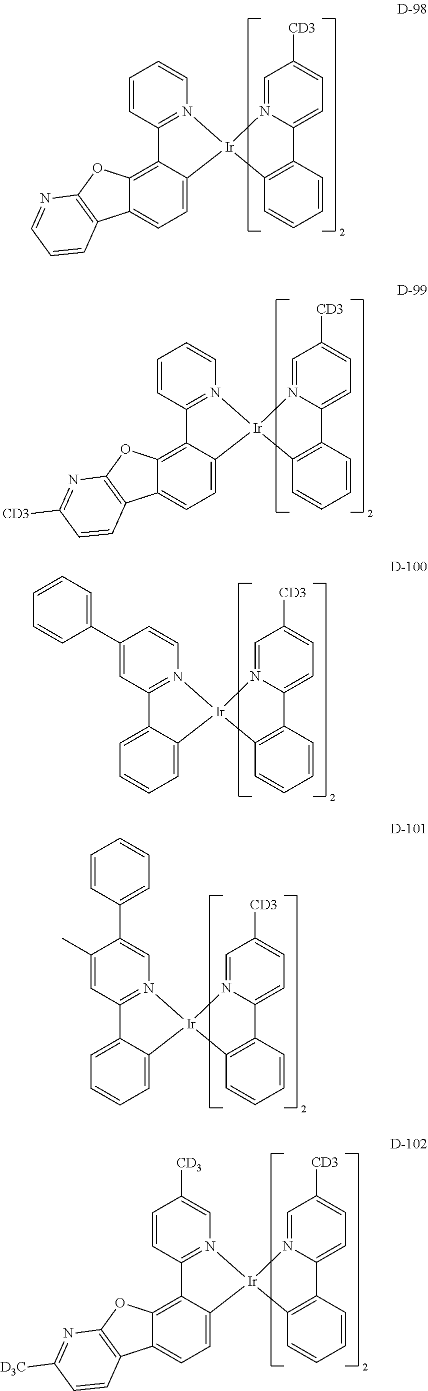

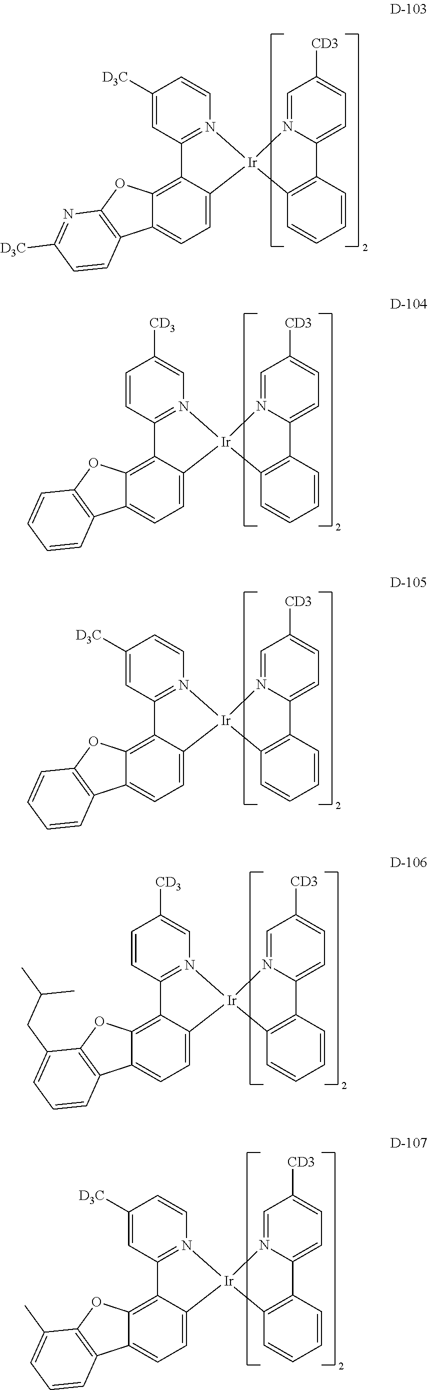

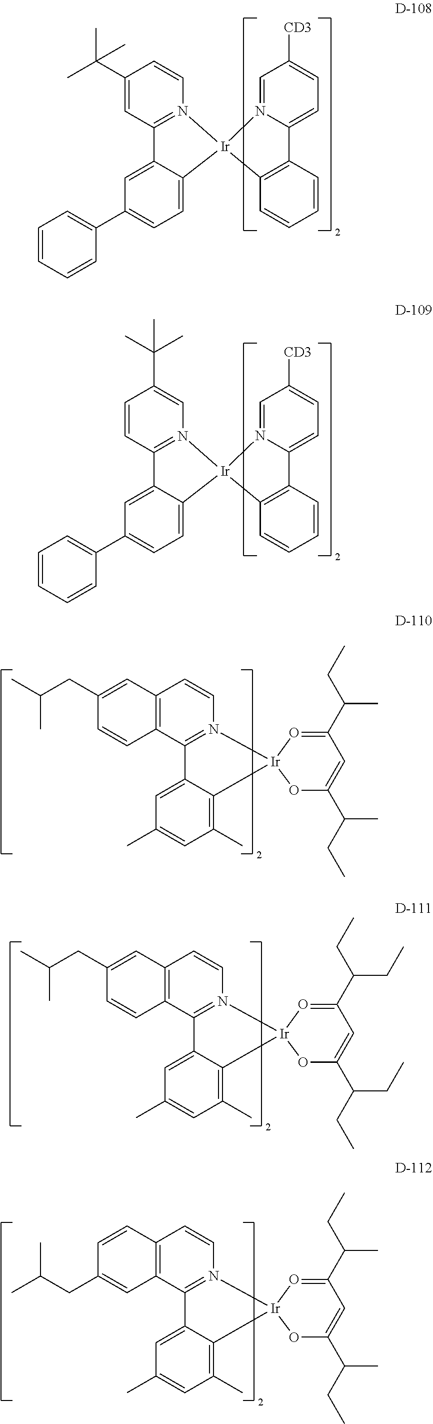

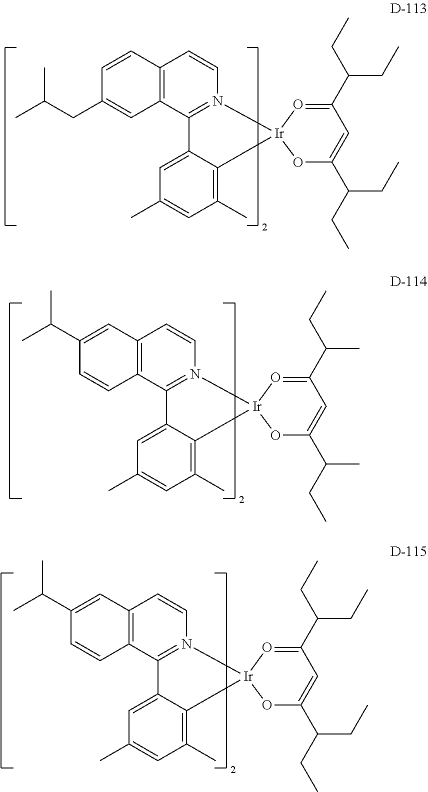

In addition, the present disclosure may provide a display system or a lighting system by using the compound represented by formula 1. That is, it is possible to produce a display system or a lighting system by using the compound of the present disclosure. Specifically, it is possible to produce a display system, e.g., a display system for smartphones, tablets, notebooks, PCs, TVs, or cars, or a lighting system, e.g., an outdoor or indoor lighting system, by using the compound of the present disclosure.

Hereinafter, the preparation method of the compound of the present disclosure, and the properties thereof will be explained in detail with reference to the representative compounds of the present disclosure. However, the present disclosure is not limited to the following examples.

### EXAMPLE 1

### Preparation of Compound H1-5

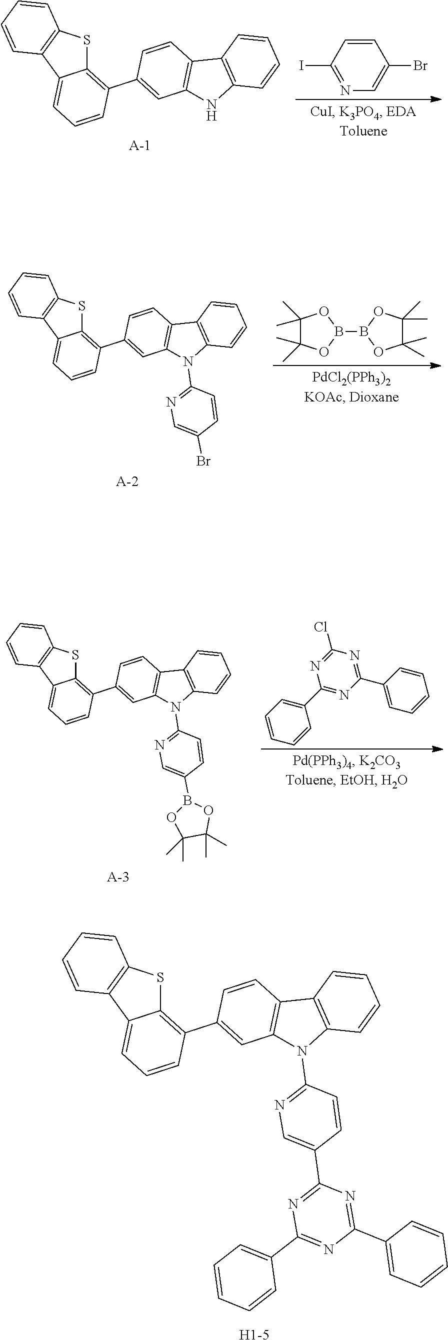

### Synthesis of Compound A-2

In a flask, 180 mL of toluene was added dropwise to compound A-1 (10.0 g, 28.6 mmol), 5-bromo-2-iodopyridine (12.2 g, 42.9 mmol), Cul (2.7 g, 14.3 mmol), ethylene diamine (EDA) (1.9 mL, 28.6 mmol), and K₃PO₄ (18.2 g, 85.8 mmol), and the mixture was stirred under reflux at 140° C. for 5 hours. After completion of the reaction, the mixture was extracted with methylene chloride (MC) and dried with MgSO₄. The residue was separated by column chromatography, and MeOH was added thereto. The resulting solid was filtered under reduced pressure to obtain compound A-2 (13.8 g, yield: 96%).

### Synthesis of Compound A-3

150 mL of 1,4-dioxane was added dropwise to compound A-2 (13.8 g, 27.4 mmol), 4,4,4′,4′,5,5,5′,5′-octamethyl-2,2′-bi(1,3,2-dioxaborolane) (8.4 g, 32.8 mmol), PdCl₂(PPh₃)₂ (0.98 g, 1.4 mmol), and KOAc (6.7 g, 68.5 mmol), and the mixture was refluxed at 130° C. for 3 hours. After completion of the reaction, an organic layer was extracted with ethyl acetate and the residual moisture was removed by using magnesium sulfate. The residue was separated by column chromatography to obtain compound A-3 (13.8 g, yield: 91%).

### Synthesis of Compound H1-5

In a flask, compound A-3 (17.0 g, 31.7 mmol), 2-chloro-4,6-dipheyl-1,3,5-triazine (10.0 g, 37.2 mmol), Pd(PPh₃)₄ (1.8 g, 1.55 mmol), and K₂CO₃ (12.9 g, 93 mmol) were dissolved in 200 mL of toluene, 100 mL of EtOH, and 100 mL of H₂O, and the mixture was refluxed at 120° C. for 3 hours. After completion of the reaction, an organic layer was extracted with ethyl acetate and the residual moisture was removed by using magnesium sulfate. The residue was separated by column chromatography to obtain compound H1-5 (7.2 g, yield: 35%).

| Compound | MW | M.P. |
|---|---|---|
| H1-5 | 657.79 | 340° C. |

Hereinafter, the properties of the organic electroluminescent device (OLED) comprising the compound according to the present disclosure will be explained in detail. However, the following examples merely illustrate the properties of an OLED according to the present disclosure in detail, but the present disclosure is not limited to the following examples.

### DEVICE EXAMPLE 1-1

### Producing an Oled Co-Deposited with a First Host Compound and a Second Host Compound According to the Present Disclosure

An OLED was produced by using the organic electroluminescent compound according to the present disclosure. A transparent electrode indium tin oxide (ITO) thin film (10 Ω/sq) on a glass substrate for an OLED (GEOMATEC CO., LTD., Japan) was subjected to an ultrasonic washing with acetone, ethanol, and distilled water, sequentially, and then was stored in isopropanol. The ITO substrate was then mounted on a substrate holder of a vacuum vapor deposition apparatus. Compound HI-1 was introduced into a cell of the vacuum vapor deposition apparatus, and then the pressure in the chamber of the apparatus was controlled to 10 ⁻⁶ torr. Thereafter, an electric current was applied to the cell to evaporate the above-introduced material, thereby forming a first hole injection layer having a thickness of 80 nm on the ITO substrate. Next, compound HI-2 was introduced into another cell of the vacuum vapor deposition apparatus and was evaporated by applying an electric current to the cell, thereby forming a second hole injection layer having a thickness of 5 nm on the first hole injection layer. Compound HT-1 was then introduced into a cell of the vacuum vapor deposition apparatus and was evaporated by applying an electric current to the cell, thereby forming a first hole transport layer having a thickness of 10 nm on the second hole injection layer. Compound HT-2 was then introduced into another cell of the vacuum vapor deposition apparatus and was evaporated by applying an electric current to the cell, thereby forming a second hole transport layer having a thickness of 30 nm on the first hole transport layer. After forming the hole injection layers and the hole transport layers, a light-emitting layer was formed thereon as follows: Compound H2-6 and compound H1-5 were respectively introduced into two cells of the vacuum vapor depositing apparatus as hosts, and compound D-48 was introduced into another cell as a dopant. The two host materials were evaporated at a rate of 1:1 and the dopant was simultaneously deposited at a different rate in a doping amount of 10 wt % based on the total amount of the hosts and dopant to form a light-emitting layer having a thickness of 40 nm on the second hole transport layer. Next, compound ET-1 and compound EI-1 were then introduced into the other two cells and evaporated at a rate of 4:6 to form an electron transport layer having a thickness of 35 nm on the light-emitting layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10 ⁻⁶ torr.

### COMPARATIVE EXAMPLE 1-1

### Producing an OLED Comprising a Conventional Host Material

An OLED was produced in the same manner as in Device Example 1-1, except that compound C-1 was used instead of compound H1-5 as a host of a light-emitting layer.

### COMPARATIVE EXAMPLE 1-2

### Producing an OLED Comprising a Conventional Host Material

An OLED was produced in the same manner as in Device Example 1-1, except that compound C-2 was used instead of compound H1-5 as a host of a light-emitting layer.

The time taken for luminance to decrease from 100% to 90% (lifetime; T90) at a luminance of 20,000 nit and at a constant current of the OLEDs produced in Device Example 1-1 and Comparative Examples 1-1 and 1-2 is provided in Table 1 below.

TABLE 1

| | First Host:Second Host | Lifetime (T90) [hr] |
|---|---|---|
| Device Example 1-1 | H1-5:H2-6 | 167.8 |
| Comparative Example | C-1:H2-6 | 132.6 |
| 1-1 | | |
| Comparative Example | C-2:H2-6 | 142.6 |
| 1-2 | | |

### DEVICE EXAMPLE 2-1

### Producing an OLED Deposited with a Host Compound According to the Present Disclosure

An OLED was produced in the same manner as in Device Example 1-1, except that a light-emitting layer was formed thereon as follows: compound H1-5 was introduced into one cell of the vacuum vapor depositing apparatus as a host, and compound D-50 was introduced into another cell as a dopant. The dopant was simultaneously deposited at a different rate in a doping amount of 10 wt % based on the total amount of the host and dopant to form a light-emitting layer having a thickness of 40 nm on the second hole transport layer.

### COMPARATIVE EXAMPLE 2-1

### Producing an OLED Comprising a Conventional Phosphorescent Host Material

An OLED was produced in the same manner as in Device Example 2-1, except that compound C-1 was used instead of compound H1-5 as a host of a light-emitting layer.

The time taken for luminance to decrease from 100% to 95% (lifetime; T95) at a luminance of 20,000 nit and at a constant current of the OLEDs produced in Device Example 2-1 and Comparative Example 2-1 is provided in Table 2 below.

TABLE 2

| | Host | Lifetime (T95) [hr] |
|---|---|---|
| Device Example 2-1 | H1-5 | 8.1 |
| Comparative Example | C-1 | 6.3 |
| 2-1 | | |

The compounds used in the Device Examples and the Comparative Examples are as follows.

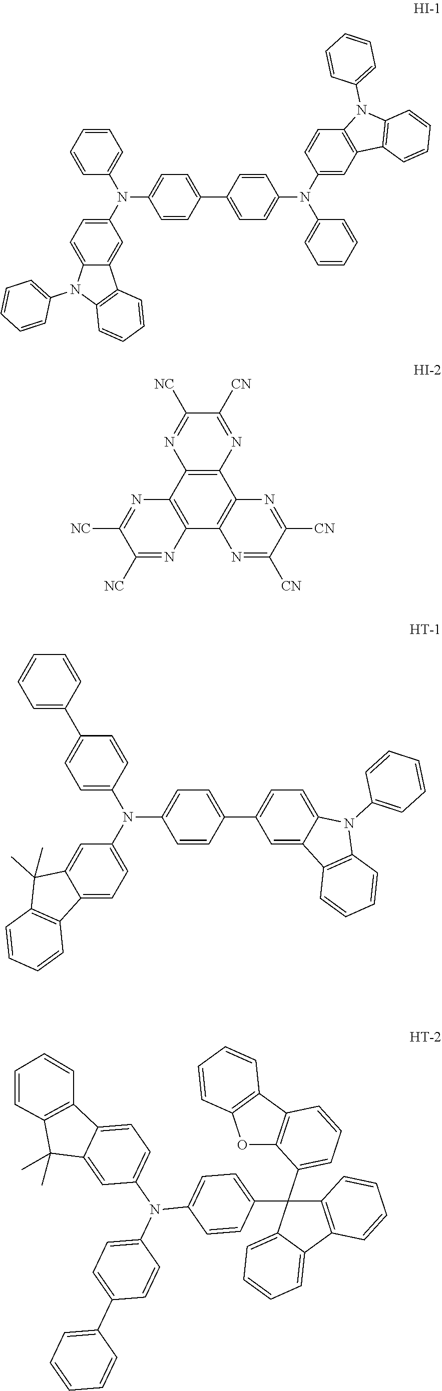 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; N#Cc1nc2c3nc(C#N)c(C#N)nc3c3nc(C#N)c(C#N)nc3c2nc1C#N ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4ccccc4)cc3)c3ccc(C4(c5cccc6c5oc5ccccc56)c5ccccc5-c5ccccc54)cc3)cc21]

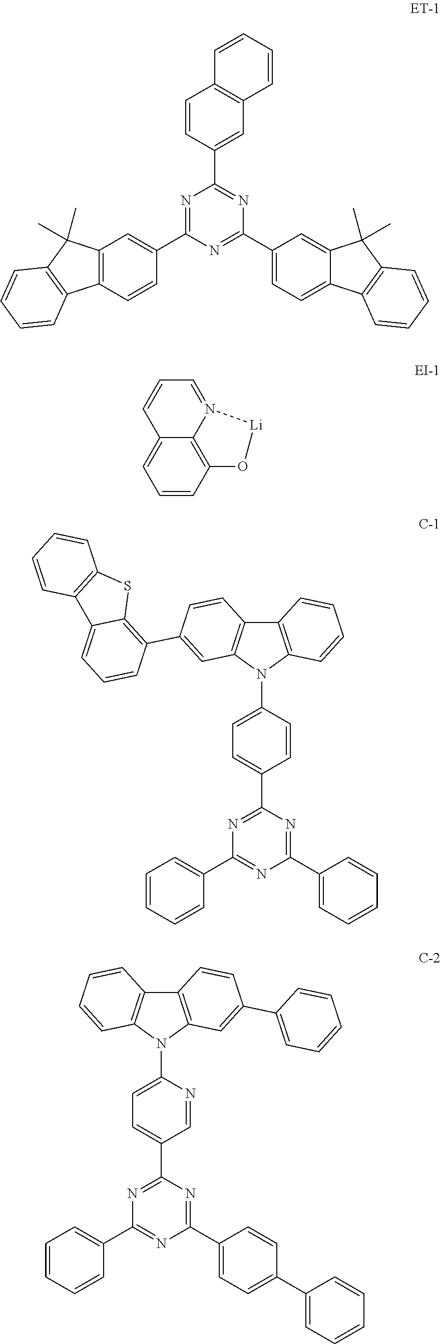

From Tables 1 and 2 above, it can be confirmed that the OLEDs comprising the compound according to the present disclosure as a host material exhibited a longer lifetime property compared to the OLEDs comprising the conventional organic electroluminescent compounds. Specifically, as shown in Table 1 above, the OLED comprising the first and second host materials according to the present disclosure and the phosphorescent dopant had an improved lifetime property compared to the OLEDs comprising the conventional host materials. In addition, as shown in Table 2 above, the OLED comprising the compound according to the present disclosure as a single host material had an improved lifetime property compared to the OLED comprising the conventional host material.

1. An organic electroluminescent compound represented by the following formula 1: 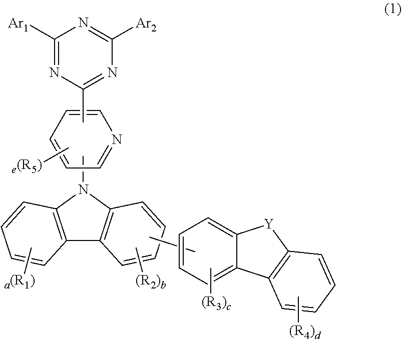
wherein
Ar₁ and Ar₂, each independently, represent a substituted or unsubstituted (C6-C30)aryl, or a substituted or unsubstituted (3- to 30-membered)heteroaryl;
Y represents O or S;
R₁ to R₅, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted (C1-C30)alkyl(C6-C30)arylamino, or a substituted or unsubstituted mono- or di- (C6-C30)arylamino; or adjacent ones of R₁ to R₅ may be linked to each other to form a ring(s); and
a and d, each independently, represent an integer of 1 to 4; b, c, and e, each independently, represent an integer of 1 to 3; where each of a to e is an integer of 2 or more, each of R₁, each of R₂, each of R₃, each of R₄, and each of R₅ may be the same or different.

2. The organic electroluminescent compound according to claim 1, wherein the formula 1 is represented by any one of the following formulas 1-1 to 1-3: 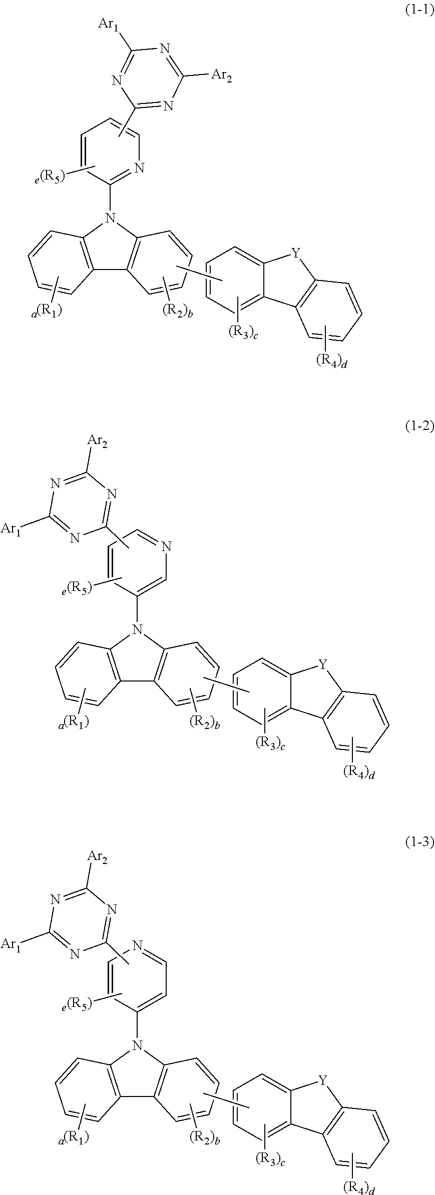
wherein Ar₁, Ar₂, Y, R₁ to R₅, and a to d are as defined in claim 1.

3. The organic electroluminescent compound according to claim 1, wherein in formula 1, 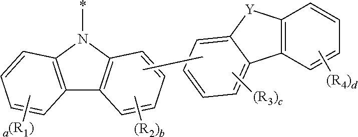
is represented by any one of the following formulas 1-4 to 1-6, in which * represents a bonding site to pyridylene in formula 1: 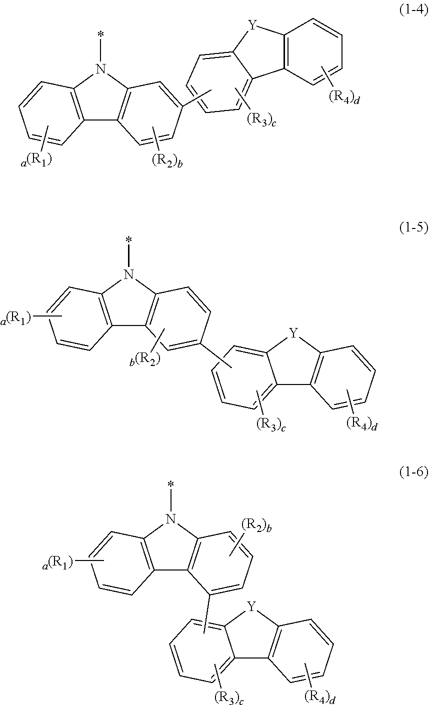
wherein Y, R₁ to R₄, and a to d are as defined in formula 1.

4. The organic electroluminescent compound according to claim 1, wherein the substituents of the substituted (C1-C30)alkyl, the substituted (C6-C30)aryl, the substituted (3- to 30-membered)heteroaryl, the substituted tri(C1-C30)alkylsilyl, the substituted di(C1-C30)alkyl(C6-C30)arylsilyl, the substituted (C1-C30)alkyldi(C6-C30)arylsilyl, the substituted tri(C6-C30)arylsilyl, the substituted mono- or di- (C1-C30)alkylamino, the substituted (C1-C30)alkyl(C6-C30)arylamino, and the substituted mono- or di- (C6-C30)arylamino in Ar₁, Ar₂, and R₁ to R₅, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di- (C1-C30)alkylamino; a mono-or di- (C6-C30)arylamino unsubstituted or substituted with a (C1-C30)alkyl(s); a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

5. The organic electroluminescent compound according to claim 1, wherein the compound represented by formula 1 is selected from the following compounds:

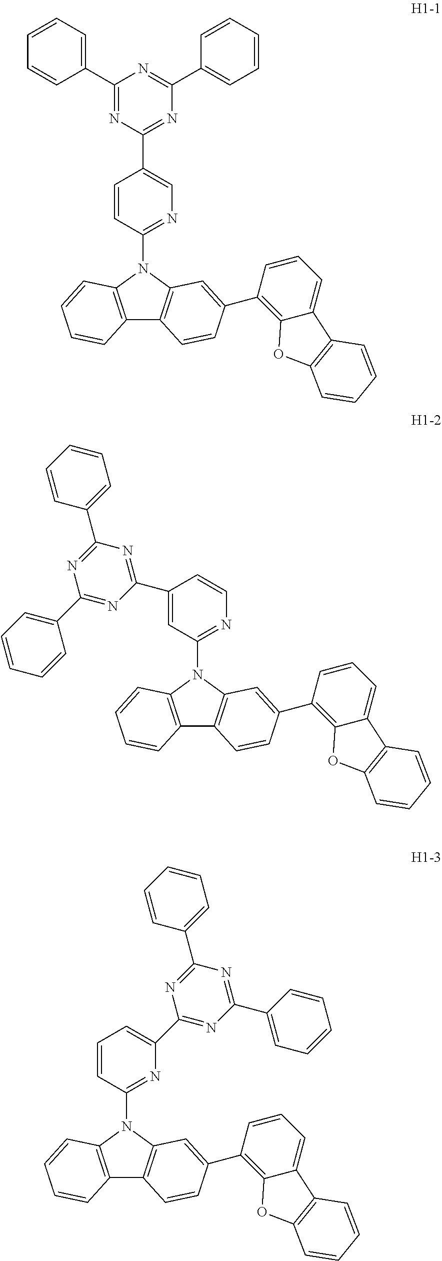 [SMILES: c1ccc(-c2nc(-c3ccccc3)nc(-c3ccc(-n4c5ccccc5c5ccc(-c6cccc7c6oc6ccccc67)cc54)nc3)n2)cc1 ; c1ccc(-c2nc(-c3ccccc3)nc(-c3ccnc(-n4c5ccccc5c5ccc(-c6cccc7c6oc6ccccc67)cc54)c3)n2)cc1 ; c1ccc(-c2nc(-c3ccccc3)nc(-c3cccc(-n4c5ccccc5c5ccc(-c6cccc7c6oc6ccccc67)cc54)n3)n2)cc1]

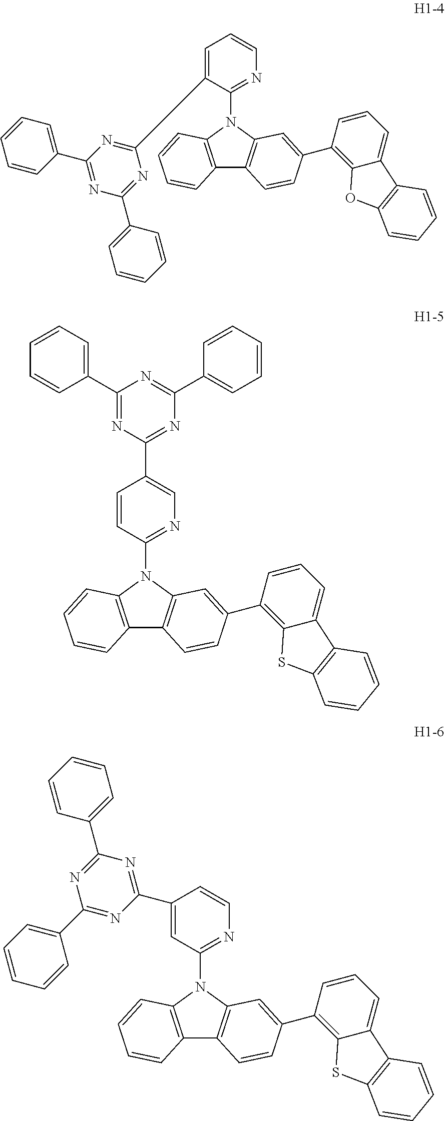 [SMILES: c1ccc(-c2nc(-c3ccccc3)nc(-c3cccnc3-n3c4ccccc4c4ccc(-c5cccc6c5oc5ccccc56)cc43)n2)cc1 ; c1ccc(-c2nc(-c3ccccc3)nc(-c3ccc(-n4c5ccccc5c5ccc(-c6cccc7c6sc6ccccc67)cc54)nc3)n2)cc1 ; c1ccc(-c2nc(-c3ccccc3)nc(-c3ccnc(-n4c5ccccc5c5ccc(-c6cccc7c6sc6ccccc67)cc54)c3)n2)cc1]

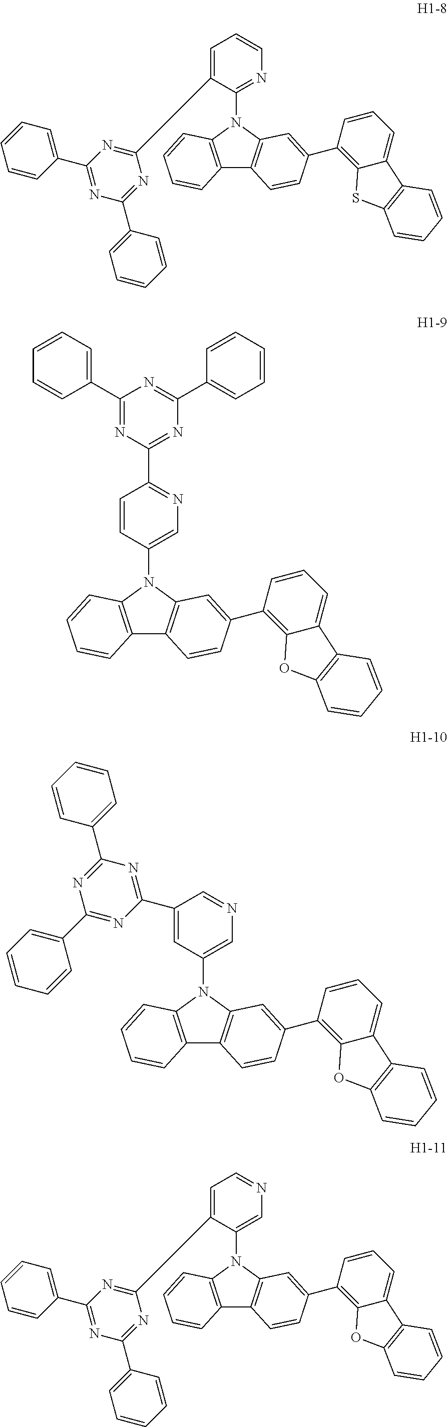 [SMILES: c1ccc(-c2nc(-c3ccccc3)nc(-c3cccnc3-n3c4ccccc4c4ccc(-c5cccc6c5sc5ccccc56)cc43)n2)cc1 ; c1ccc(-c2nc(-c3ccccc3)nc(-c3ccc(-n4c5ccccc5c5ccc(-c6cccc7c6oc6ccccc67)cc54)cn3)n2)cc1 ; c1ccc(-c2nc(-c3ccccc3)nc(-c3cncc(-n4c5ccccc5c5ccc(-c6cccc7c6oc6ccccc67)cc54)c3)n2)cc1 ; c1ccc(-c2nc(-c3ccccc3)nc(-c3ccncc3-n3c4ccccc4c4ccc(-c5cccc6c5oc5ccccc56)cc43)n2)cc1]

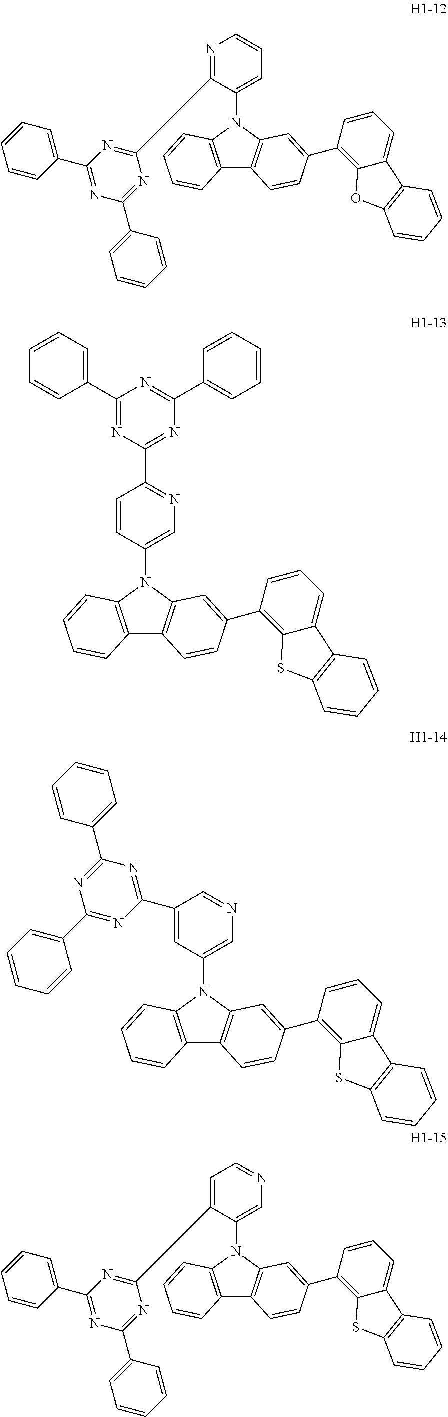 [SMILES: c1ccc(-c2nc(-c3ccccc3)nc(-c3ncccc3-n3c4ccccc4c4ccc(-c5cccc6c5oc5ccccc56)cc43)n2)cc1 ; c1ccc(-c2nc(-c3ccccc3)nc(-c3ccc(-n4c5ccccc5c5ccc(-c6cccc7c6sc6ccccc67)cc54)cn3)n2)cc1 ; c1ccc(-c2nc(-c3ccccc3)nc(-c3cncc(-n4c5ccccc5c5ccc(-c6cccc7c6sc6ccccc67)cc54)c3)n2)cc1 ; c1ccc(-c2nc(-c3ccccc3)nc(-c3ccncc3-n3c4ccccc4c4ccc(-c5cccc6c5sc5ccccc56)cc43)n2)cc1]

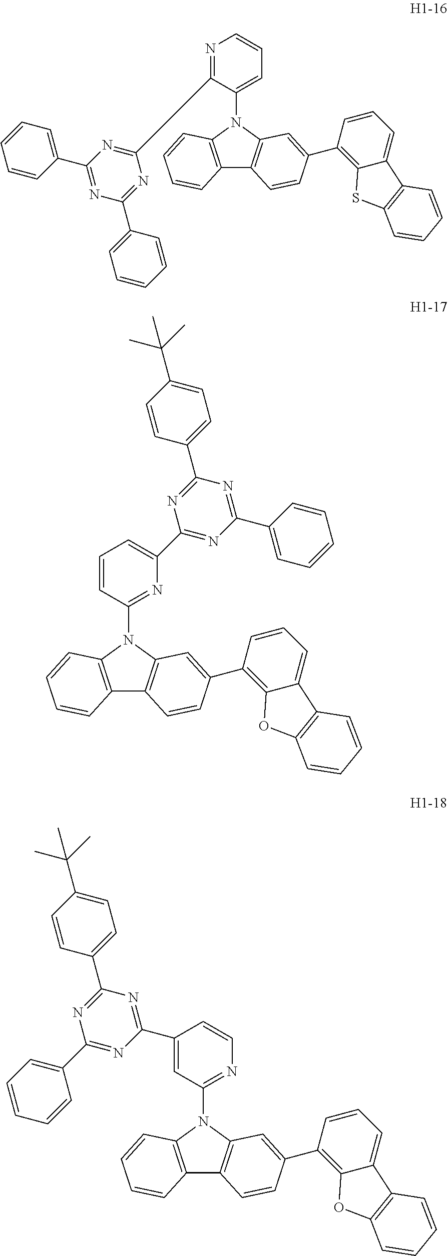

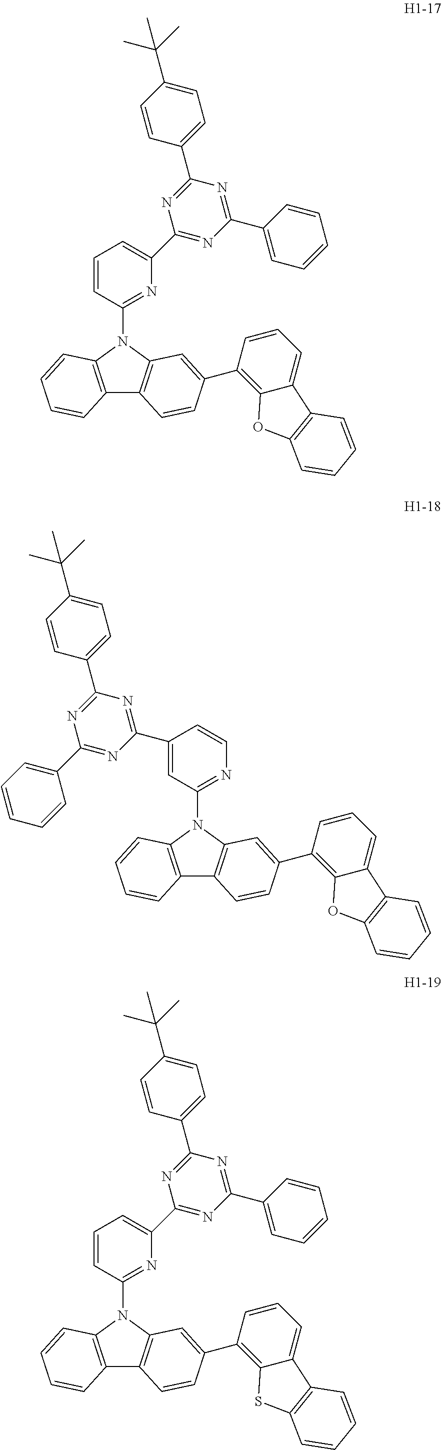 [SMILES: CC(C)(C)c1ccc(-c2nc(-c3ccccc3)nc(-c3cccc(-n4c5ccccc5c5ccc(-c6cccc7c6oc6ccccc67)cc54)n3)n2)cc1 ; CC(C)(C)c1ccc(-c2nc(-c3ccccc3)nc(-c3ccnc(-n4c5ccccc5c5ccc(-c6cccc7c6oc6ccccc67)cc54)c3)n2)cc1 ; CC(C)(C)c1ccc(-c2nc(-c3ccccc3)nc(-c3cccc(-n4c5ccccc5c5ccc(-c6cccc7c6sc6ccccc67)cc54)n3)n2)cc1]

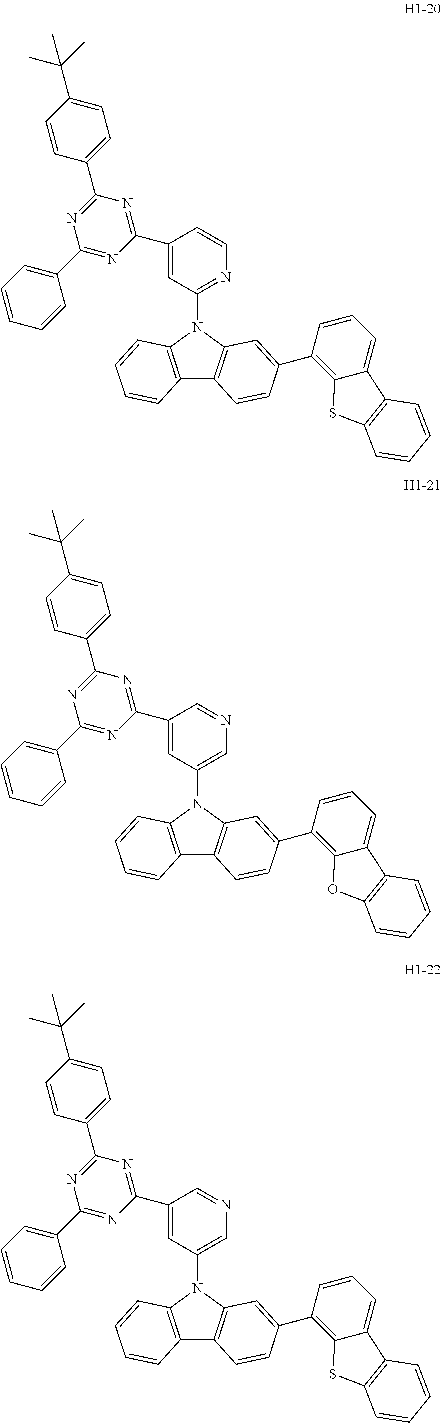 [SMILES: CC(C)(C)c1ccc(-c2nc(-c3ccccc3)nc(-c3ccnc(-n4c5ccccc5c5ccc(-c6cccc7c6sc6ccccc67)cc54)c3)n2)cc1 ; CC(C)(C)c1ccc(-c2nc(-c3ccccc3)nc(-c3cncc(-n4c5ccccc5c5ccc(-c6cccc7c6oc6ccccc67)cc54)c3)n2)cc1 ; CC(C)(C)c1ccc(-c2nc(-c3ccccc3)nc(-c3cncc(-n4c5ccccc5c5ccc(-c6cccc7c6sc6ccccc67)cc54)c3)n2)cc1]

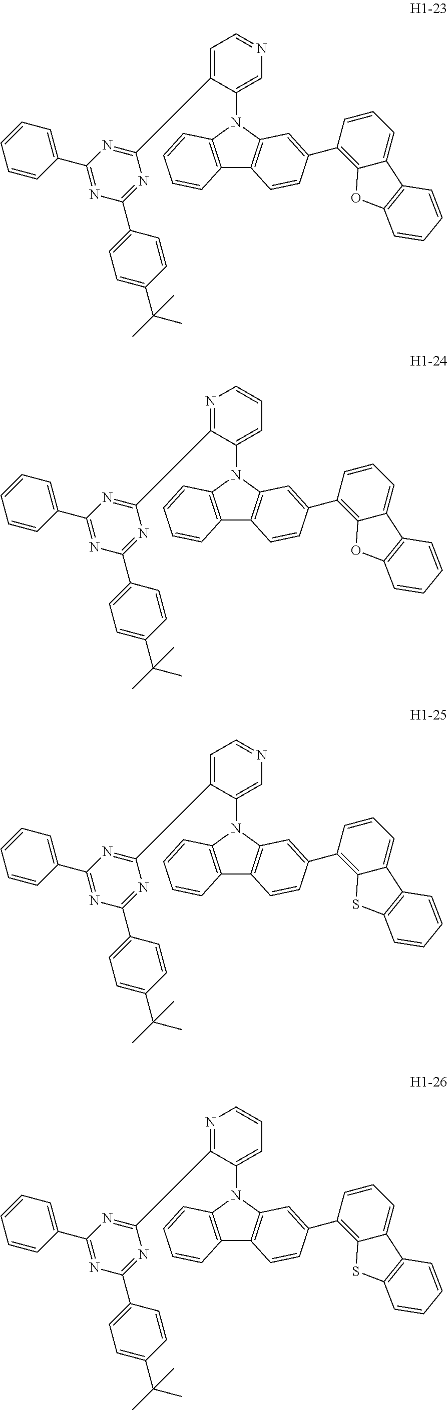 [SMILES: CC(C)(C)c1ccc(-c2nc(-c3ccccc3)nc(-c3ccncc3-n3c4ccccc4c4ccc(-c5cccc6c5oc5ccccc56)cc43)n2)cc1 ; CC(C)(C)c1ccc(-c2nc(-c3ccccc3)nc(-c3ncccc3-n3c4ccccc4c4ccc(-c5cccc6c5oc5ccccc56)cc43)n2)cc1 ; CC(C)(C)c1ccc(-c2nc(-c3ccccc3)nc(-c3ccncc3-n3c4ccccc4c4ccc(-c5cccc6c5sc5ccccc56)cc43)n2)cc1 ; CC(C)(C)c1ccc(-c2nc(-c3ccccc3)nc(-c3ncccc3-n3c4ccccc4c4ccc(-c5cccc6c5sc5ccccc56)cc43)n2)cc1]

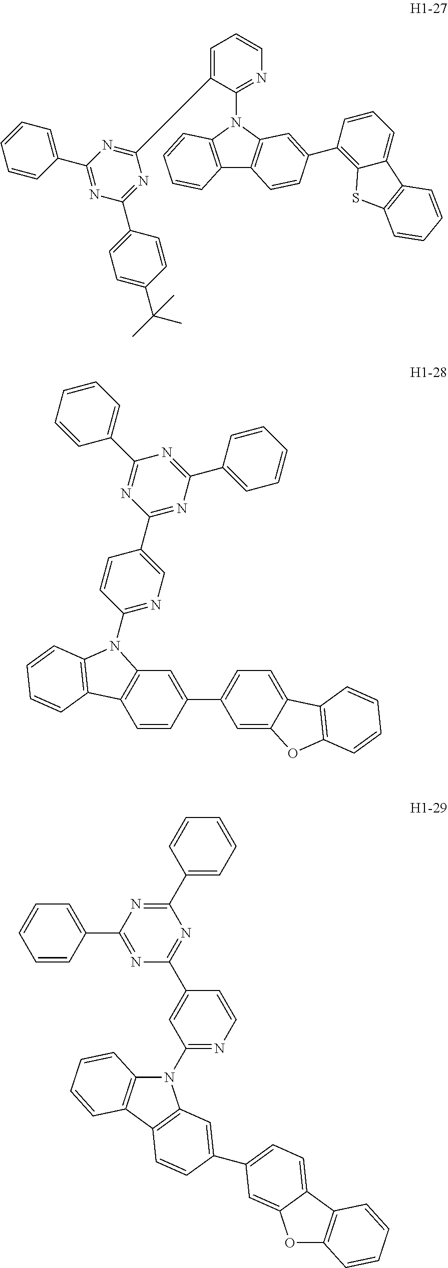 [SMILES: CC(C)(C)c1ccc(-c2nc(-c3ccccc3)nc(-c3cccnc3-n3c4ccccc4c4ccc(-c5cccc6c5sc5ccccc56)cc43)n2)cc1 ; c1ccc(-c2nc(-c3ccccc3)nc(-c3ccc(-n4c5ccccc5c5ccc(-c6ccc7c(c6)oc6ccccc67)cc54)nc3)n2)cc1 ; c1ccc(-c2nc(-c3ccccc3)nc(-c3ccnc(-n4c5ccccc5c5ccc(-c6ccc7c(c6)oc6ccccc67)cc54)c3)n2)cc1]

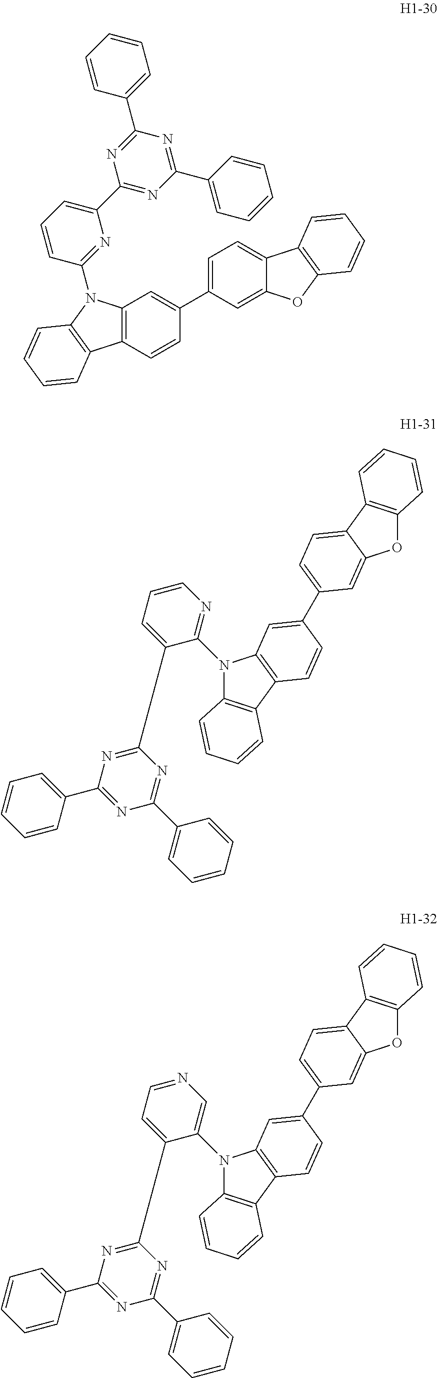 [SMILES: c1ccc(-c2nc(-c3ccccc3)nc(-c3cccc(-n4c5ccccc5c5ccc(-c6ccc7c(c6)oc6ccccc67)cc54)n3)n2)cc1 ; c1ccc(-c2nc(-c3ccccc3)nc(-c3cccnc3-n3c4ccccc4c4ccc(-c5ccc6c(c5)oc5ccccc56)cc43)n2)cc1 ; c1ccc(-c2nc(-c3ccccc3)nc(-c3ccncc3-n3c4ccccc4c4ccc(-c5ccc6c(c5)oc5ccccc56)cc43)n2)cc1]

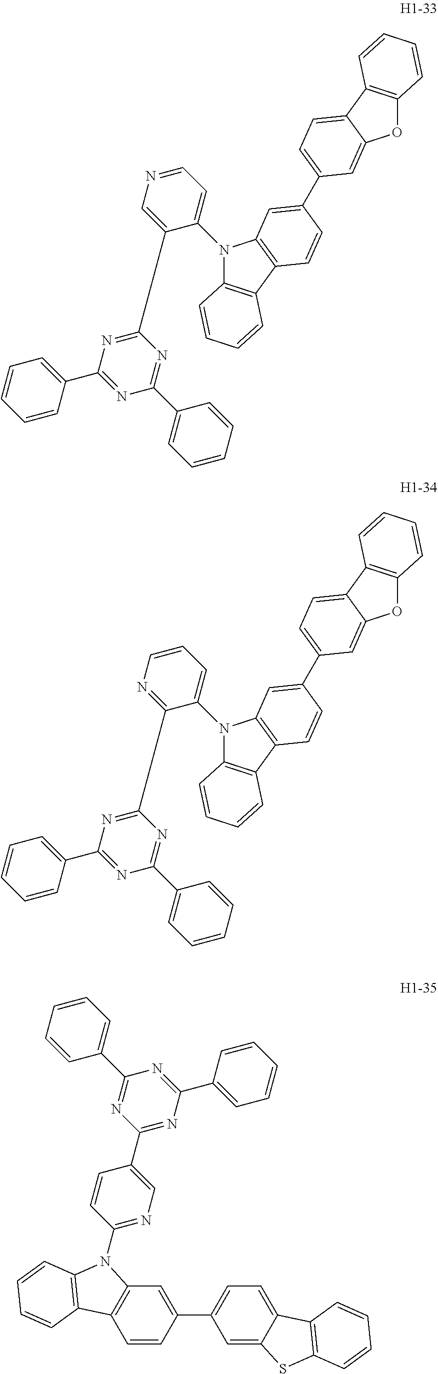 [SMILES: c1ccc(-c2nc(-c3ccccc3)nc(-c3cnccc3-n3c4ccccc4c4ccc(-c5ccc6c(c5)oc5ccccc56)cc43)n2)cc1 ; c1ccc(-c2nc(-c3ccccc3)nc(-c3ncccc3-n3c4ccccc4c4ccc(-c5ccc6c(c5)oc5ccccc56)cc43)n2)cc1 ; c1ccc(-c2nc(-c3ccccc3)nc(-c3ccc(-n4c5ccccc5c5ccc(-c6ccc7c(c6)sc6ccccc67)cc54)nc3)n2)cc1]

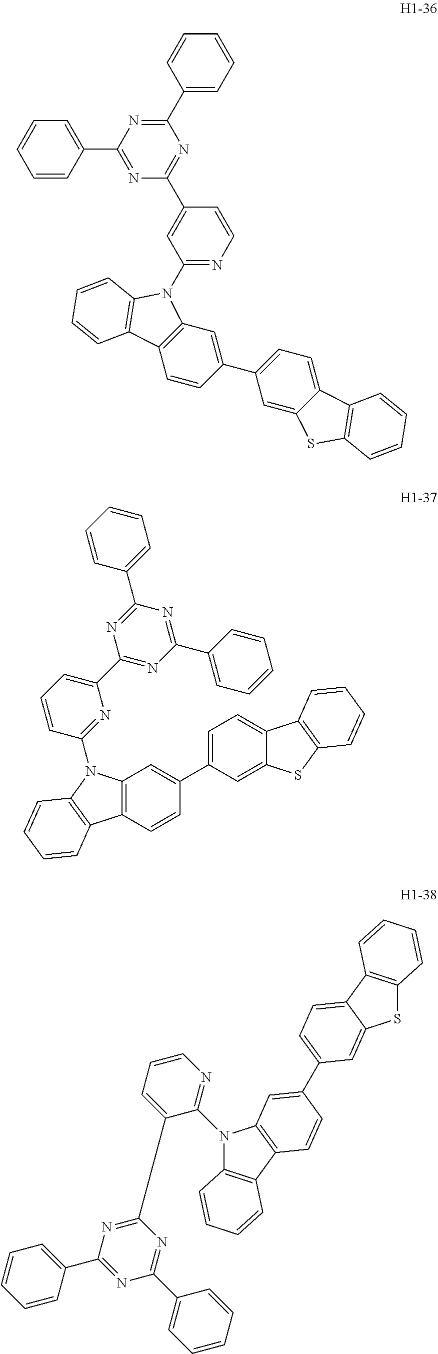 [SMILES: c1ccc(-c2nc(-c3ccccc3)nc(-c3ccnc(-n4c5ccccc5c5ccc(-c6ccc7c(c6)sc6ccccc67)cc54)c3)n2)cc1 ; c1ccc(-c2nc(-c3ccccc3)nc(-c3cccc(-n4c5ccccc5c5ccc(-c6ccc7c(c6)sc6ccccc67)cc54)n3)n2)cc1 ; c1ccc(-c2nc(-c3ccccc3)nc(-c3cccnc3-n3c4ccccc4c4ccc(-c5ccc6c(c5)sc5ccccc56)cc43)n2)cc1]

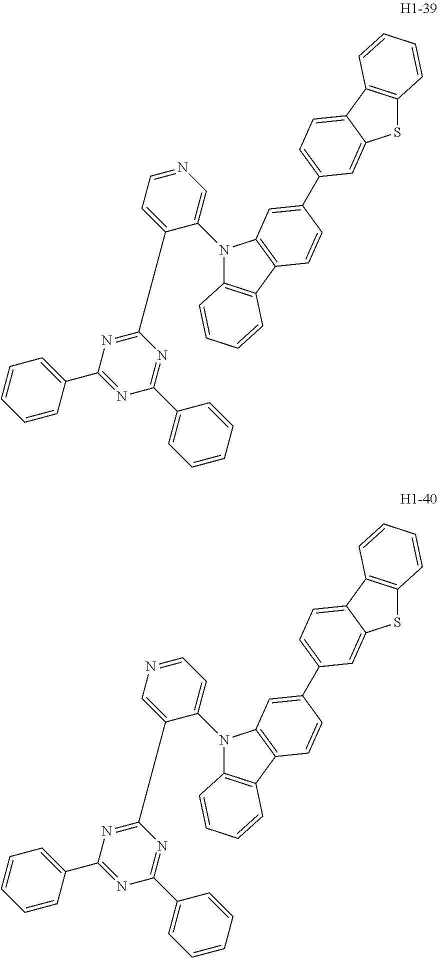 [SMILES: c1ccc(-c2nc(-c3ccccc3)nc(-c3ccncc3-n3c4ccccc4c4ccc(-c5ccc6c(c5)sc5ccccc56)cc43)n2)cc1 ; c1ccc(-c2nc(-c3ccccc3)nc(-c3cnccc3-n3c4ccccc4c4ccc(-c5ccc6c(c5)sc5ccccc56)cc43)n2)cc1]

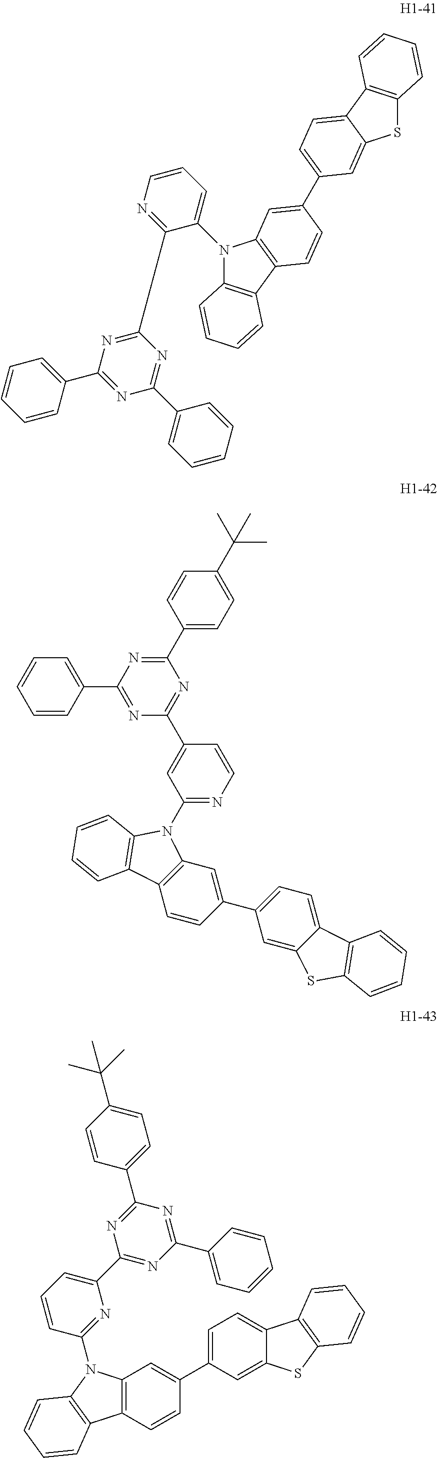 [SMILES: c1ccc(-c2nc(-c3ccccc3)nc(-c3ncccc3-n3c4ccccc4c4ccc(-c5ccc6c(c5)sc5ccccc56)cc43)n2)cc1 ; CC(C)(C)c1ccc(-c2nc(-c3ccccc3)nc(-c3ccnc(-n4c5ccccc5c5ccc(-c6ccc7c(c6)sc6ccccc67)cc54)c3)n2)cc1 ; CC(C)(C)c1ccc(-c2nc(-c3ccccc3)nc(-c3cccc(-n4c5ccccc5c5ccc(-c6ccc7c(c6)sc6ccccc67)cc54)n3)n2)cc1]

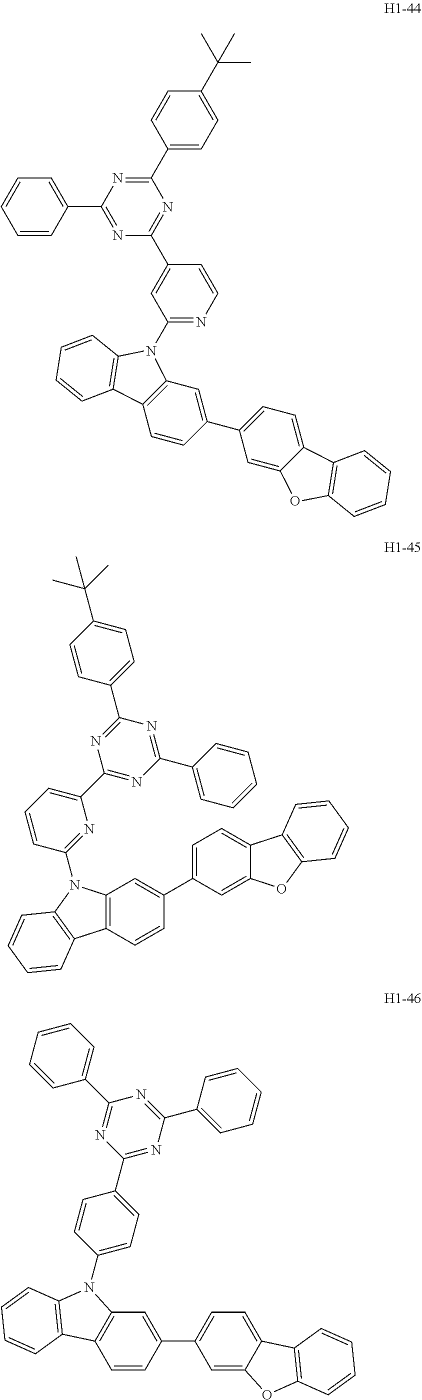 [SMILES: CC(C)(C)c1ccc(-c2nc(-c3ccccc3)nc(-c3ccnc(-n4c5ccccc5c5ccc(-c6ccc7c(c6)oc6ccccc67)cc54)c3)n2)cc1 ; CC(C)(C)c1ccc(-c2nc(-c3ccccc3)nc(-c3cccc(-n4c5ccccc5c5ccc(-c6ccc7c(c6)oc6ccccc67)cc54)n3)n2)cc1 ; c1ccc(-c2nc(-c3ccccc3)nc(-c3ccc(-n4c5ccccc5c5ccc(-c6ccc7c(c6)oc6ccccc67)cc54)cc3)n2)cc1]

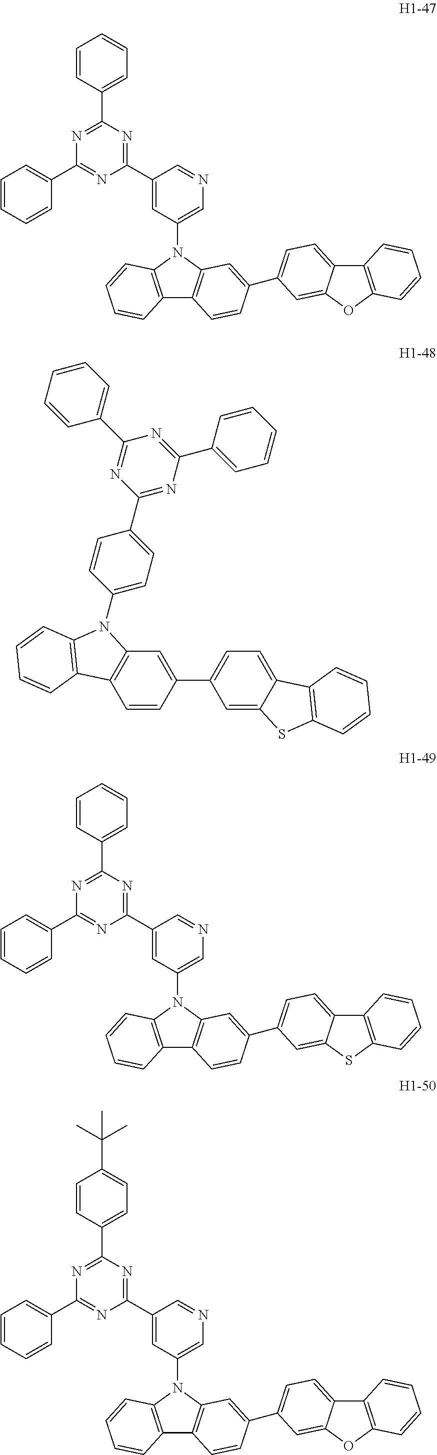 [SMILES: c1ccc(-c2nc(-c3ccccc3)nc(-c3cncc(-n4c5ccccc5c5ccc(-c6ccc7c(c6)oc6ccccc67)cc54)c3)n2)cc1 ; c1ccc(-c2nc(-c3ccccc3)nc(-c3ccc(-n4c5ccccc5c5ccc(-c6ccc7c(c6)sc6ccccc67)cc54)cc3)n2)cc1 ; c1ccc(-c2nc(-c3ccccc3)nc(-c3cncc(-n4c5ccccc5c5ccc(-c6ccc7c(c6)sc6ccccc67)cc54)c3)n2)cc1 ; CC(C)(C)c1ccc(-c2nc(-c3ccccc3)nc(-c3cncc(-n4c5ccccc5c5ccc(-c6ccc7c(c6)oc6ccccc67)cc54)c3)n2)cc1]

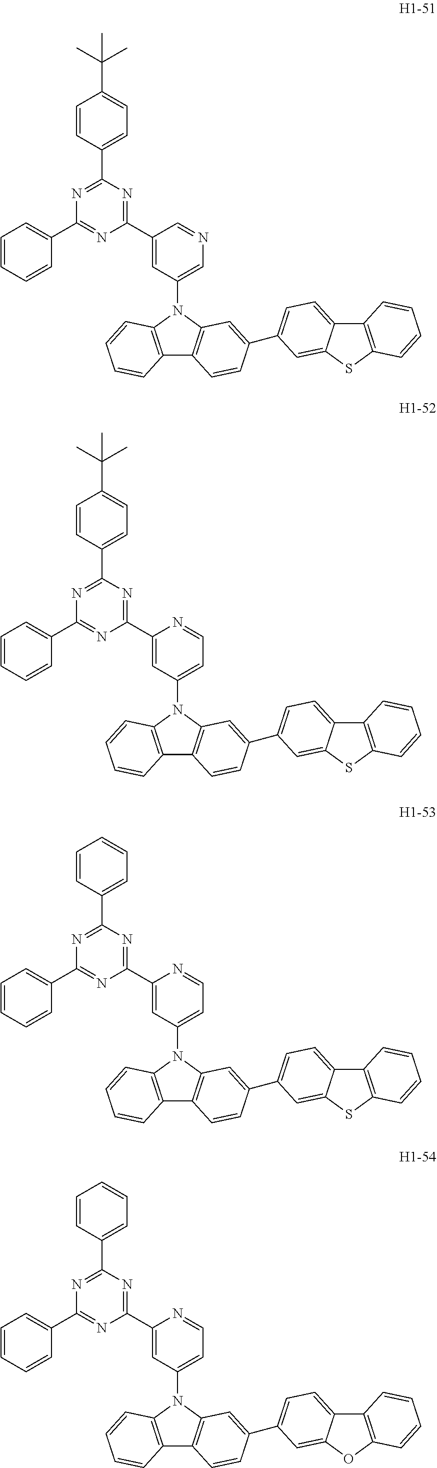 [SMILES: CC(C)(C)c1ccc(-c2nc(-c3ccccc3)nc(-c3cncc(-n4c5ccccc5c5ccc(-c6ccc7c(c6)sc6ccccc67)cc54)c3)n2)cc1 ; CC(C)(C)c1ccc(-c2nc(-c3ccccc3)nc(-c3cc(-n4c5ccccc5c5ccc(-c6ccc7c(c6)sc6ccccc67)cc54)ccn3)n2)cc1 ; c1ccc(-c2nc(-c3ccccc3)nc(-c3cc(-n4c5ccccc5c5ccc(-c6ccc7c(c6)sc6ccccc67)cc54)ccn3)n2)cc1 ; c1ccc(-c2nc(-c3ccccc3)nc(-c3cc(-n4c5ccccc5c5ccc(-c6ccc7c(c6)oc6ccccc67)cc54)ccn3)n2)cc1]

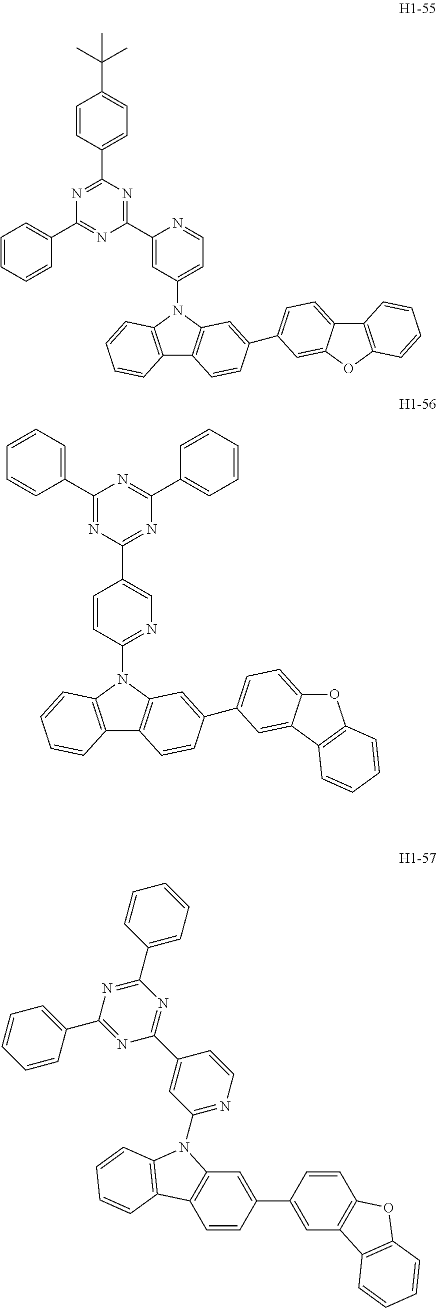 [SMILES: CC(C)(C)c1ccc(-c2nc(-c3ccccc3)nc(-c3cc(-n4c5ccccc5c5ccc(-c6ccc7c(c6)oc6ccccc67)cc54)ccn3)n2)cc1 ; c1ccc(-c2nc(-c3ccccc3)nc(-c3ccc(-n4c5ccccc5c5ccc(-c6ccc7oc8ccccc8c7c6)cc54)nc3)n2)cc1 ; c1ccc(-c2nc(-c3ccccc3)nc(-c3ccnc(-n4c5ccccc5c5ccc(-c6ccc7oc8ccccc8c7c6)cc54)c3)n2)cc1]

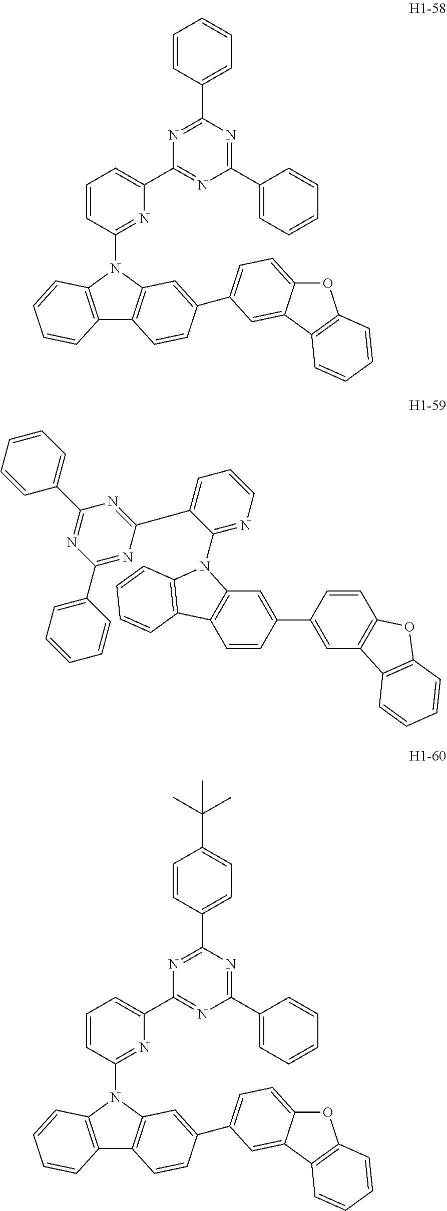 [SMILES: c1ccc(-c2nc(-c3ccccc3)nc(-c3cccc(-n4c5ccccc5c5ccc(-c6ccc7oc8ccccc8c7c6)cc54)n3)n2)cc1 ; c1ccc(-c2nc(-c3ccccc3)nc(-c3cccnc3-n3c4ccccc4c4ccc(-c5ccc6oc7ccccc7c6c5)cc43)n2)cc1 ; CC(C)(C)c1ccc(-c2nc(-c3ccccc3)nc(-c3cccc(-n4c5ccccc5c5ccc(-c6ccc7oc8ccccc8c7c6)cc54)n3)n2)cc1]

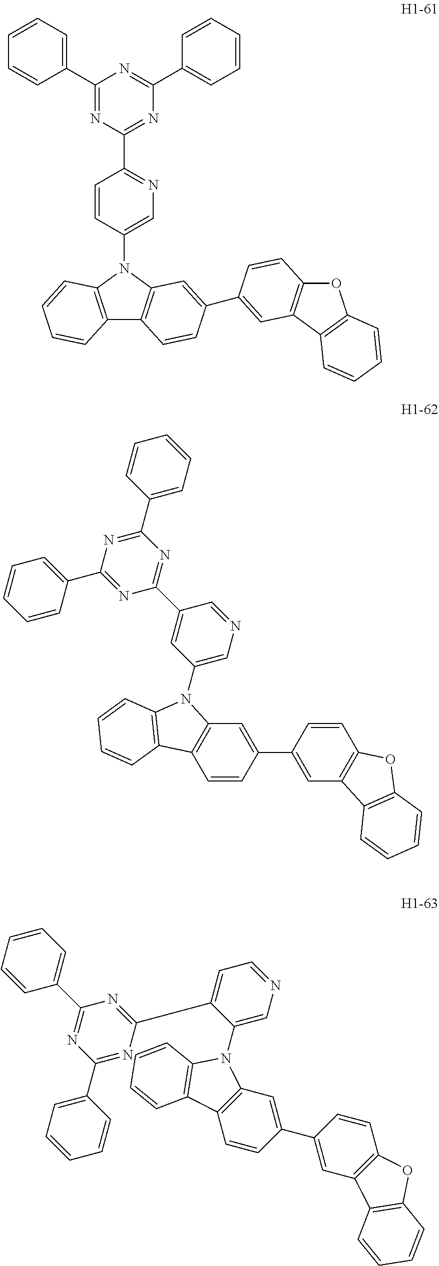 [SMILES: c1ccc(-c2nc(-c3ccccc3)nc(-c3ccc(-n4c5ccccc5c5ccc(-c6ccc7oc8ccccc8c7c6)cc54)cn3)n2)cc1 ; c1ccc(-c2nc(-c3ccccc3)nc(-c3cncc(-n4c5ccccc5c5ccc(-c6ccc7oc8ccccc8c7c6)cc54)c3)n2)cc1 ; c1ccc(-c2nc(-c3ccccc3)nc(-c3ccncc3-n3c4ccccc4c4ccc(-c5ccc6oc7ccccc7c6c5)cc43)n2)cc1]

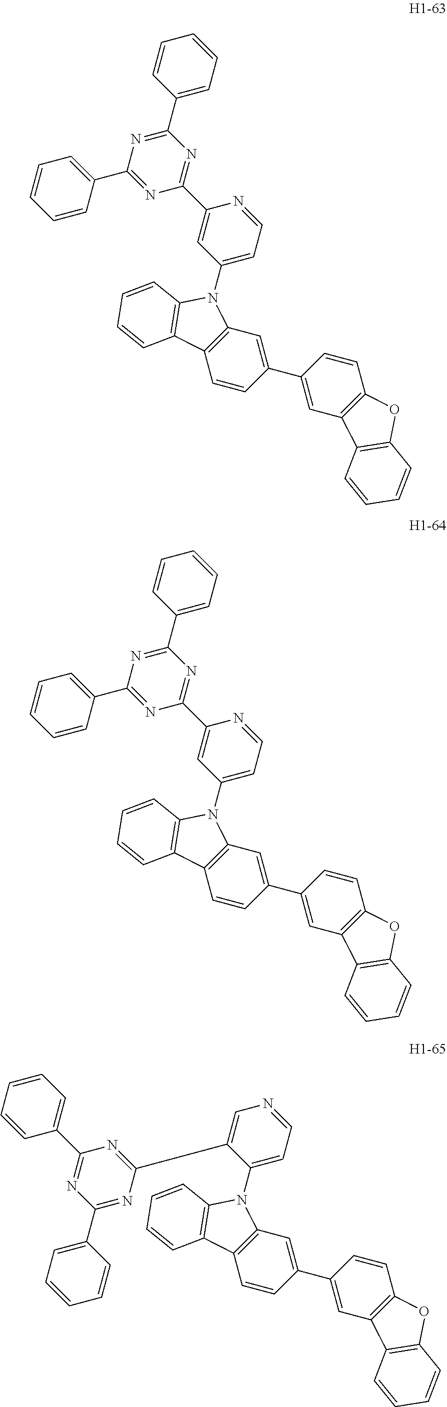 [SMILES: c1ccc(-c2nc(-c3ccccc3)nc(-c3cc(-n4c5ccccc5c5ccc(-c6ccc7oc8ccccc8c7c6)cc54)ccn3)n2)cc1 ; c1ccc(-c2nc(-c3ccccc3)nc(-c3cc(-n4c5ccccc5c5ccc(-c6ccc7oc8ccccc8c7c6)cc54)ccn3)n2)cc1 ; c1ccc(-c2nc(-c3ccccc3)nc(-c3cnccc3-n3c4ccccc4c4ccc(-c5ccc6oc7ccccc7c6c5)cc43)n2)cc1]

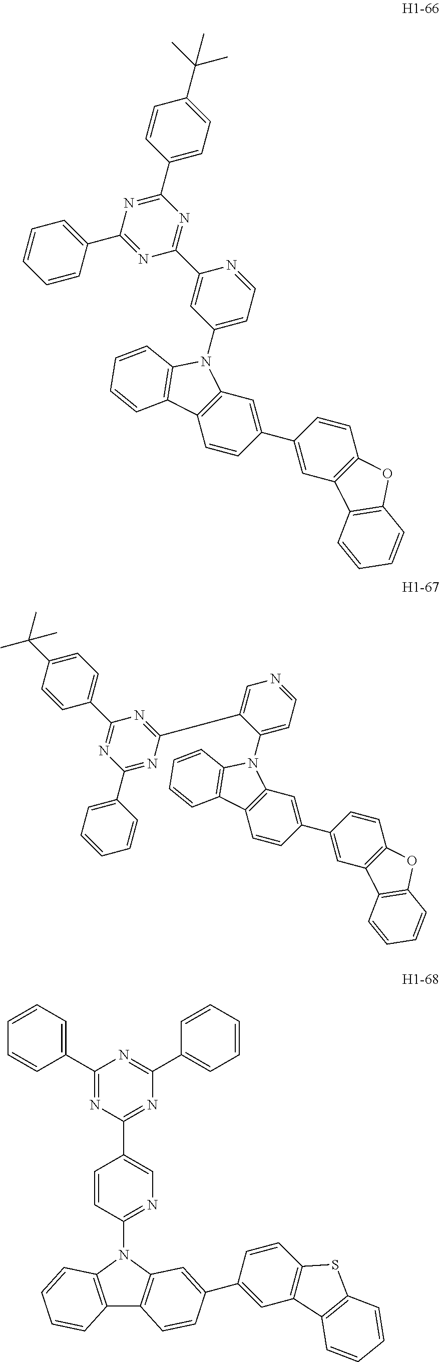 [SMILES: CC(C)(C)c1ccc(-c2nc(-c3ccccc3)nc(-c3cc(-n4c5ccccc5c5ccc(-c6ccc7oc8ccccc8c7c6)cc54)ccn3)n2)cc1 ; CC(C)(C)c1ccc(-c2nc(-c3ccccc3)nc(-c3cnccc3-n3c4ccccc4c4ccc(-c5ccc6oc7ccccc7c6c5)cc43)n2)cc1 ; c1ccc(-c2nc(-c3ccccc3)nc(-c3ccc(-n4c5ccccc5c5ccc(-c6ccc7sc8ccccc8c7c6)cc54)nc3)n2)cc1]

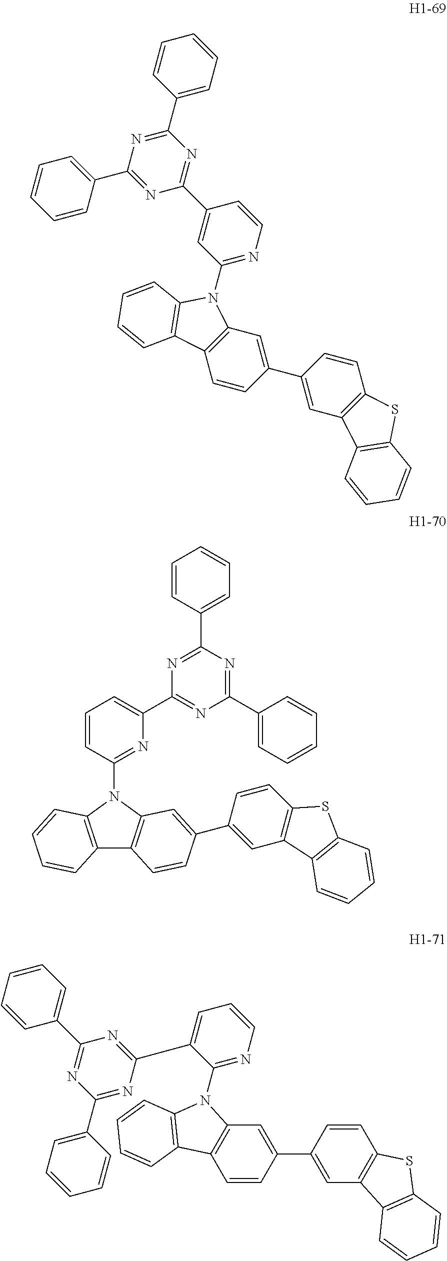 [SMILES: c1ccc(-c2nc(-c3ccccc3)nc(-c3ccnc(-n4c5ccccc5c5ccc(-c6ccc7sc8ccccc8c7c6)cc54)c3)n2)cc1 ; c1ccc(-c2nc(-c3ccccc3)nc(-c3cccc(-n4c5ccccc5c5ccc(-c6ccc7sc8ccccc8c7c6)cc54)n3)n2)cc1 ; c1ccc(-c2nc(-c3ccccc3)nc(-c3cccnc3-n3c4ccccc4c4ccc(-c5ccc6sc7ccccc7c6c5)cc43)n2)cc1]

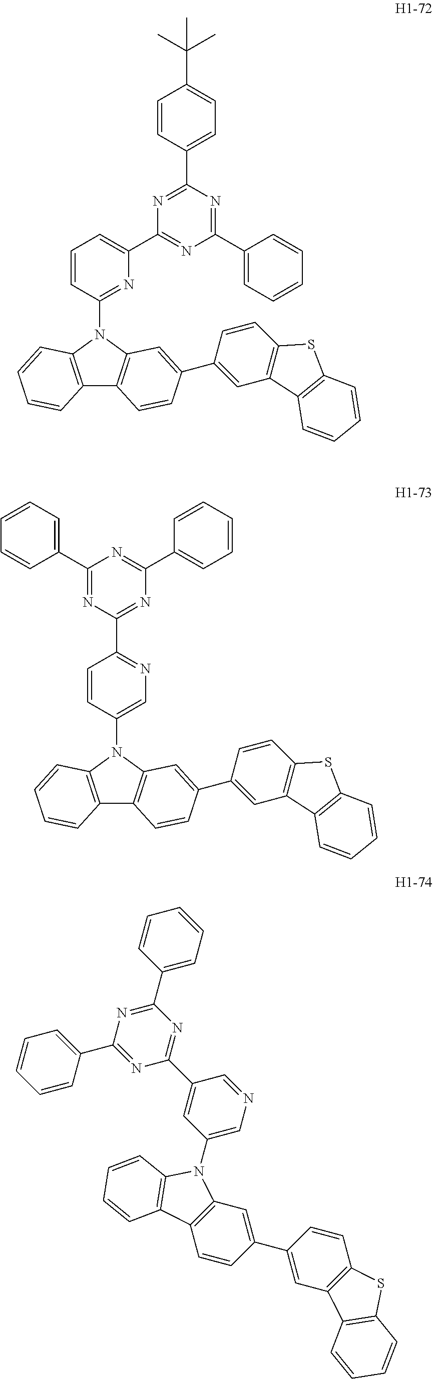 [SMILES: CC(C)(C)c1ccc(-c2nc(-c3ccccc3)nc(-c3cccc(-n4c5ccccc5c5ccc(-c6ccc7sc8ccccc8c7c6)cc54)n3)n2)cc1 ; c1ccc(-c2nc(-c3ccccc3)nc(-c3ccc(-n4c5ccccc5c5ccc(-c6ccc7sc8ccccc8c7c6)cc54)cn3)n2)cc1 ; c1ccc(-c2nc(-c3ccccc3)nc(-c3cncc(-n4c5ccccc5c5ccc(-c6ccc7sc8ccccc8c7c6)cc54)c3)n2)cc1]

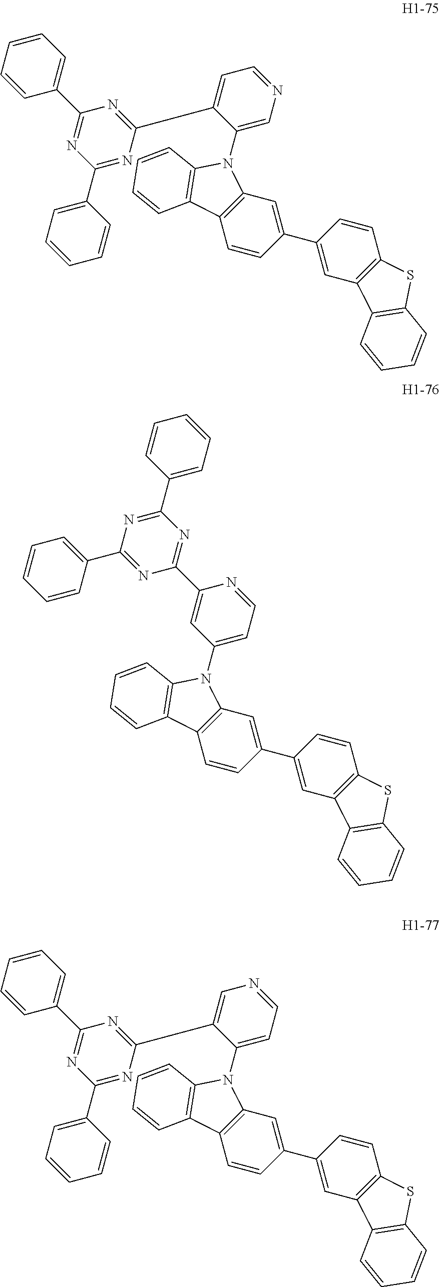 [SMILES: c1ccc(-c2nc(-c3ccccc3)nc(-c3ccncc3-n3c4ccccc4c4ccc(-c5ccc6sc7ccccc7c6c5)cc43)n2)cc1 ; c1ccc(-c2nc(-c3ccccc3)nc(-c3cc(-n4c5ccccc5c5ccc(-c6ccc7sc8ccccc8c7c6)cc54)ccn3)n2)cc1 ; c1ccc(-c2nc(-c3ccccc3)nc(-c3cnccc3-n3c4ccccc4c4ccc(-c5ccc6sc7ccccc7c6c5)cc43)n2)cc1]

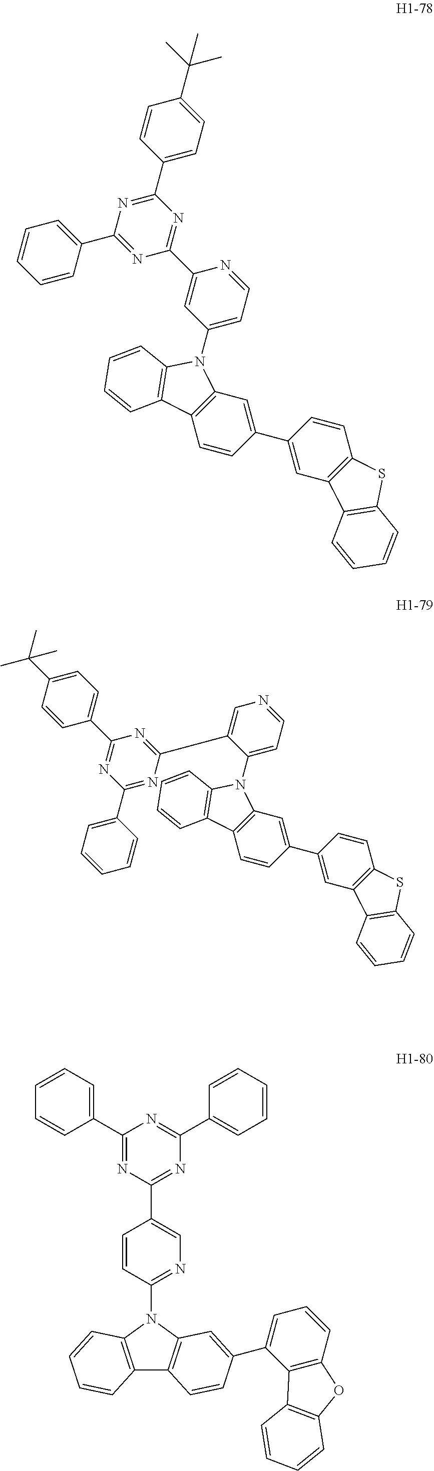 [SMILES: CC(C)(C)c1ccc(-c2nc(-c3ccccc3)nc(-c3cc(-n4c5ccccc5c5ccc(-c6ccc7sc8ccccc8c7c6)cc54)ccn3)n2)cc1 ; CC(C)(C)c1ccc(-c2nc(-c3ccccc3)nc(-c3cnccc3-n3c4ccccc4c4ccc(-c5ccc6sc7ccccc7c6c5)cc43)n2)cc1 ; c1ccc(-c2nc(-c3ccccc3)nc(-c3ccc(-n4c5ccccc5c5ccc(-c6cccc7oc8ccccc8c67)cc54)nc3)n2)cc1]

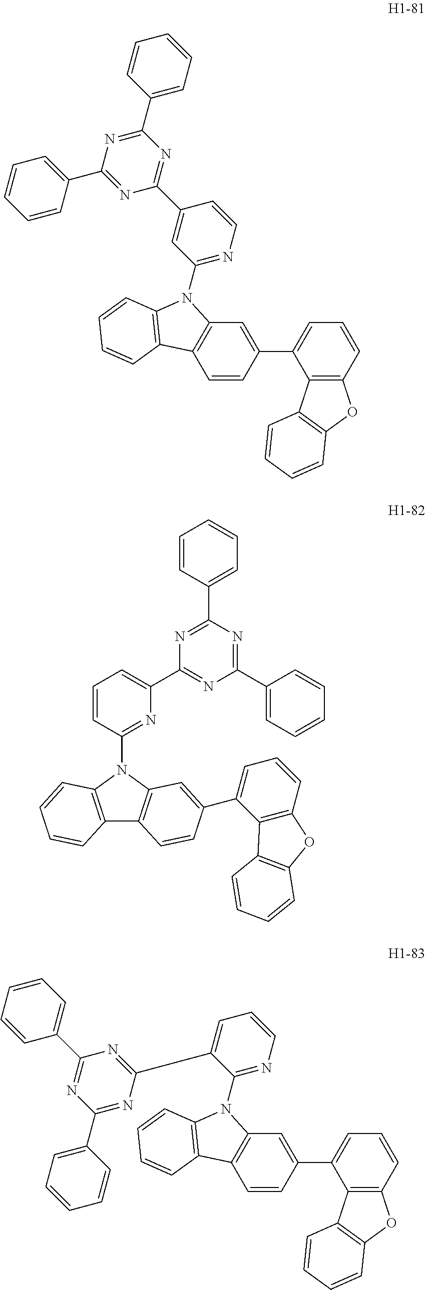 [SMILES: c1ccc(-c2nc(-c3ccccc3)nc(-c3ccnc(-n4c5ccccc5c5ccc(-c6cccc7oc8ccccc8c67)cc54)c3)n2)cc1 ; c1ccc(-c2nc(-c3ccccc3)nc(-c3cccc(-n4c5ccccc5c5ccc(-c6cccc7oc8ccccc8c67)cc54)n3)n2)cc1 ; c1ccc(-c2nc(-c3ccccc3)nc(-c3cccnc3-n3c4ccccc4c4ccc(-c5cccc6oc7ccccc7c56)cc43)n2)cc1]

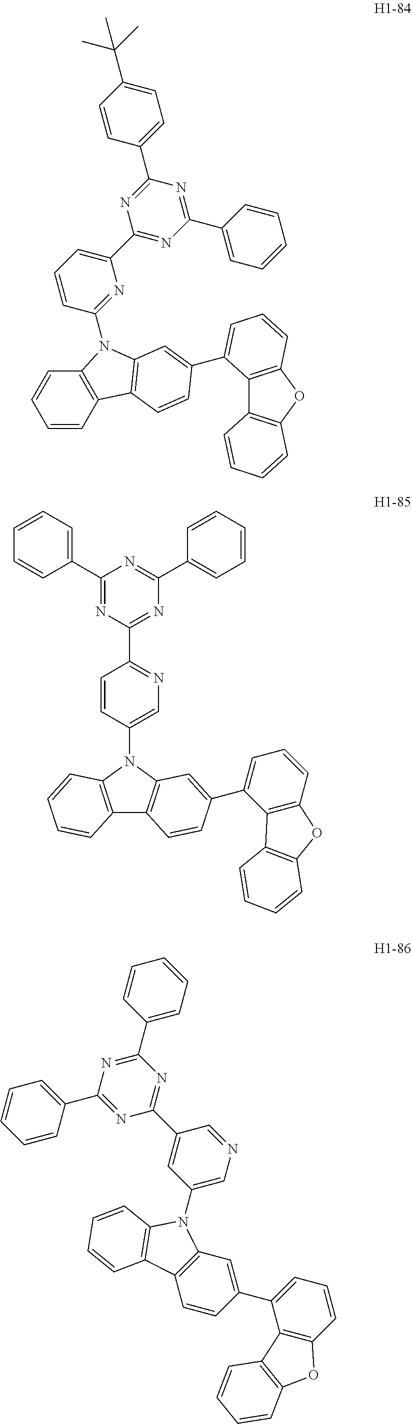

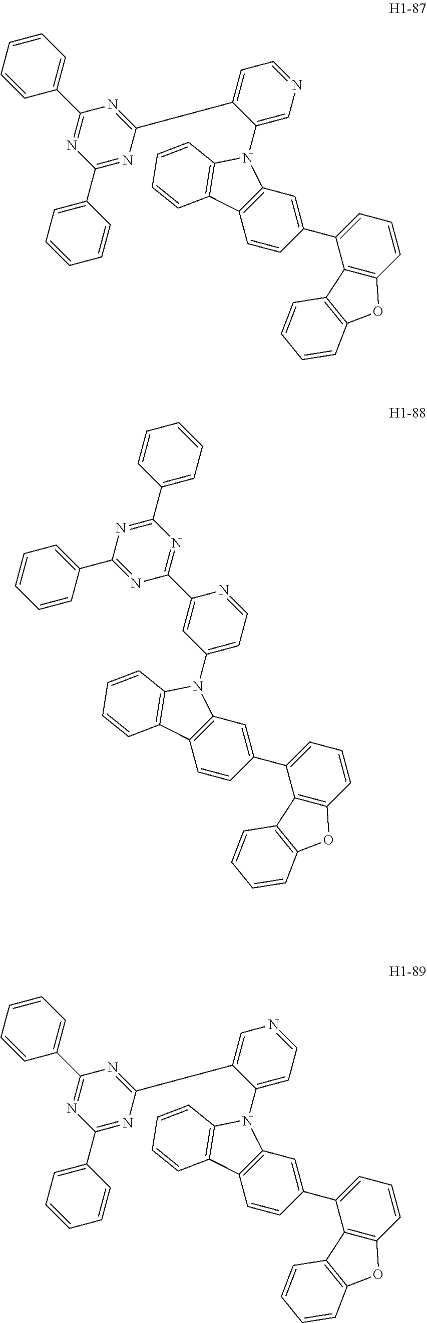 [SMILES: c1ccc(-c2nc(-c3ccccc3)nc(-c3ccncc3-n3c4ccccc4c4ccc(-c5cccc6oc7ccccc7c56)cc43)n2)cc1 ; c1ccc(-c2nc(-c3ccccc3)nc(-c3cc(-n4c5ccccc5c5ccc(-c6cccc7oc8ccccc8c67)cc54)ccn3)n2)cc1 ; c1ccc(-c2nc(-c3ccccc3)nc(-c3cnccc3-n3c4ccccc4c4ccc(-c5cccc6oc7ccccc7c56)cc43)n2)cc1]

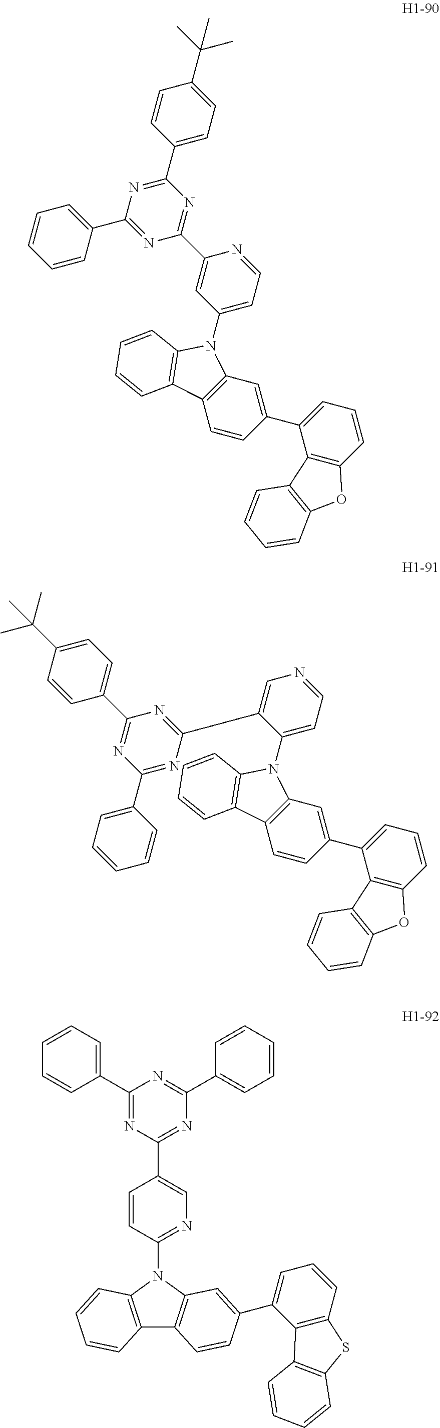 [SMILES: CC(C)(C)c1ccc(-c2nc(-c3ccccc3)nc(-c3cc(-n4c5ccccc5c5ccc(-c6cccc7oc8ccccc8c67)cc54)ccn3)n2)cc1 ; CC(C)(C)c1ccc(-c2nc(-c3ccccc3)nc(-c3cnccc3-n3c4ccccc4c4ccc(-c5cccc6oc7ccccc7c56)cc43)n2)cc1 ; c1ccc(-c2nc(-c3ccccc3)nc(-c3ccc(-n4c5ccccc5c5ccc(-c6cccc7sc8ccccc8c67)cc54)nc3)n2)cc1]

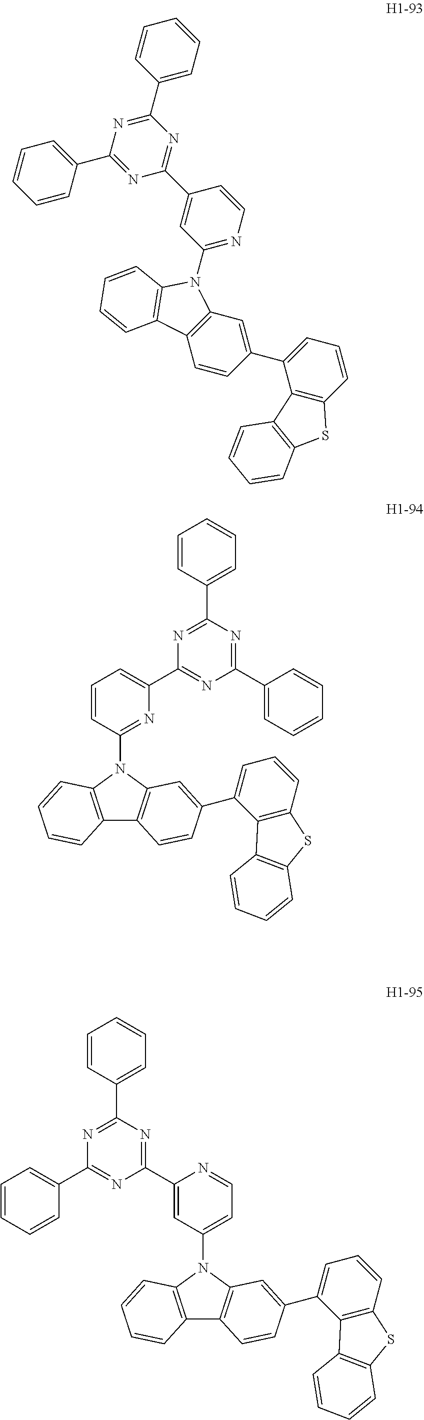 [SMILES: c1ccc(-c2nc(-c3ccccc3)nc(-c3ccnc(-n4c5ccccc5c5ccc(-c6cccc7sc8ccccc8c67)cc54)c3)n2)cc1 ; c1ccc(-c2nc(-c3ccccc3)nc(-c3cccc(-n4c5ccccc5c5ccc(-c6cccc7sc8ccccc8c67)cc54)n3)n2)cc1 ; c1ccc(-c2nc(-c3ccccc3)nc(-c3cc(-n4c5ccccc5c5ccc(-c6cccc7sc8ccccc8c67)cc54)ccn3)n2)cc1]

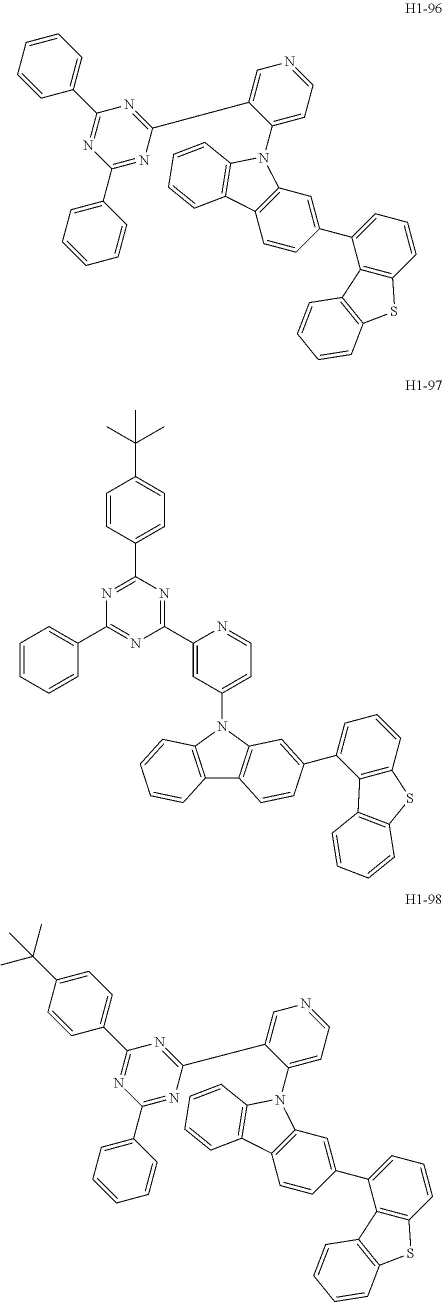 [SMILES: c1ccc(-c2nc(-c3ccccc3)nc(-c3cnccc3-n3c4ccccc4c4ccc(-c5cccc6sc7ccccc7c56)cc43)n2)cc1 ; CC(C)(C)c1ccc(-c2nc(-c3ccccc3)nc(-c3cc(-n4c5ccccc5c5ccc(-c6cccc7sc8ccccc8c67)cc54)ccn3)n2)cc1 ; CC(C)(C)c1ccc(-c2nc(-c3ccccc3)nc(-c3cnccc3-n3c4ccccc4c4ccc(-c5cccc6sc7ccccc7c56)cc43)n2)cc1]

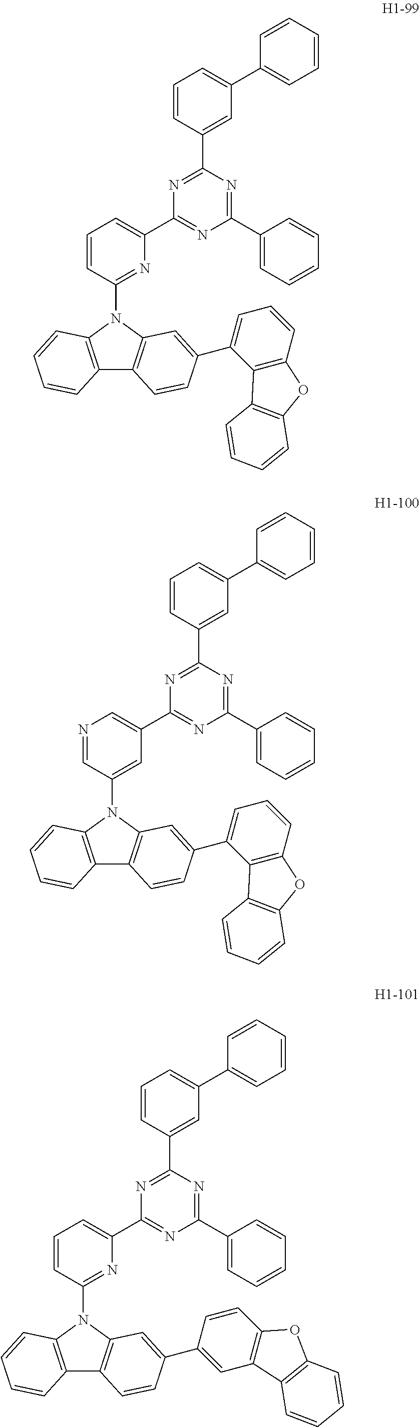 [SMILES: c1ccc(-c2cccc(-c3nc(-c4ccccc4)nc(-c4cccc(-n5c6ccccc6c6ccc(-c7cccc8oc9ccccc9c78)cc65)n4)n3)c2)cc1 ; c1ccc(-c2cccc(-c3nc(-c4ccccc4)nc(-c4cncc(-n5c6ccccc6c6ccc(-c7cccc8oc9ccccc9c78)cc65)c4)n3)c2)cc1 ; c1ccc(-c2cccc(-c3nc(-c4ccccc4)nc(-c4cccc(-n5c6ccccc6c6ccc(-c7ccc8oc9ccccc9c8c7)cc65)n4)n3)c2)cc1]

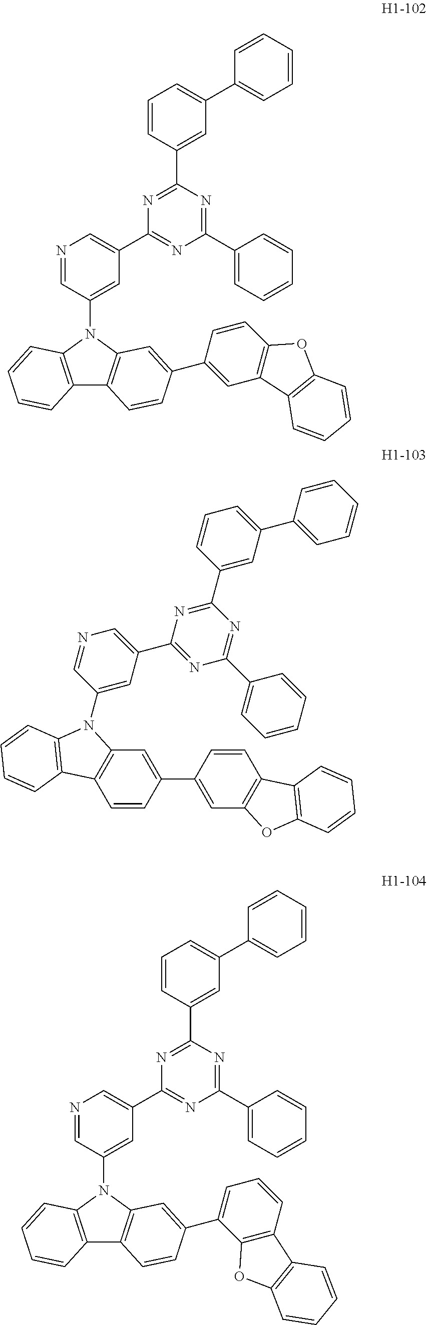

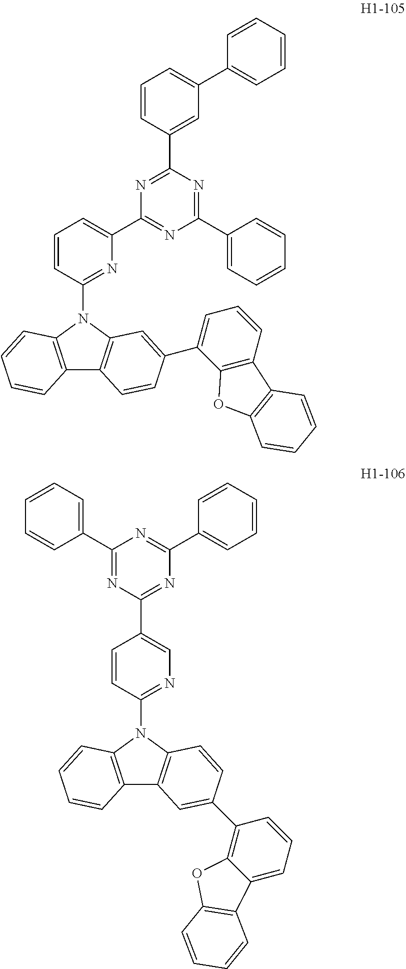 [SMILES: c1ccc(-c2cccc(-c3nc(-c4ccccc4)nc(-c4cccc(-n5c6ccccc6c6ccc(-c7cccc8c7oc7ccccc78)cc65)n4)n3)c2)cc1 ; c1ccc(-c2nc(-c3ccccc3)nc(-c3ccc(-n4c5ccccc5c5cc(-c6cccc7c6oc6ccccc67)ccc54)nc3)n2)cc1]

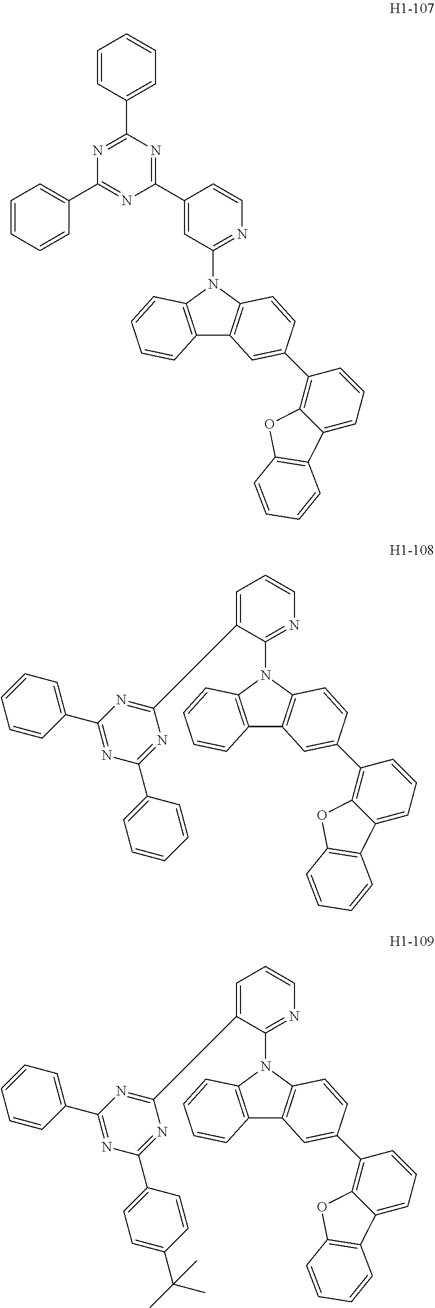 [SMILES: c1ccc(-c2nc(-c3ccccc3)nc(-c3ccnc(-n4c5ccccc5c5cc(-c6cccc7c6oc6ccccc67)ccc54)c3)n2)cc1 ; c1ccc(-c2nc(-c3ccccc3)nc(-c3cccnc3-n3c4ccccc4c4cc(-c5cccc6c5oc5ccccc56)ccc43)n2)cc1 ; CC(C)(C)c1ccc(-c2nc(-c3ccccc3)nc(-c3cccnc3-n3c4ccccc4c4cc(-c5cccc6c5oc5ccccc56)ccc43)n2)cc1]

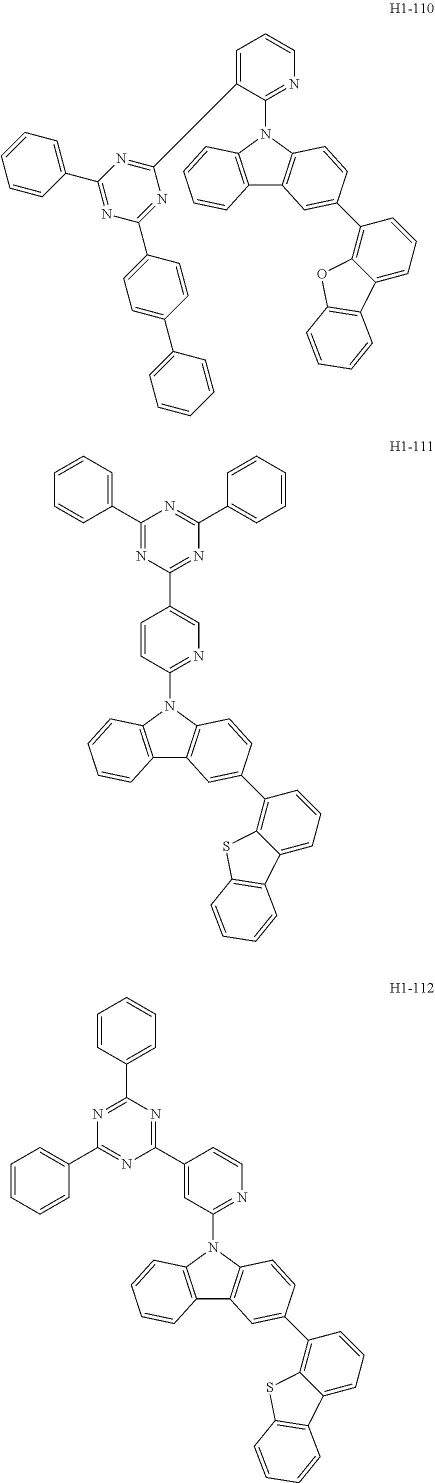 [SMILES: c1ccc(-c2ccc(-c3nc(-c4ccccc4)nc(-c4cccnc4-n4c5ccccc5c5cc(-c6cccc7c6oc6ccccc67)ccc54)n3)cc2)cc1 ; c1ccc(-c2nc(-c3ccccc3)nc(-c3ccc(-n4c5ccccc5c5cc(-c6cccc7c6sc6ccccc67)ccc54)nc3)n2)cc1 ; c1ccc(-c2nc(-c3ccccc3)nc(-c3ccnc(-n4c5ccccc5c5cc(-c6cccc7c6sc6ccccc67)ccc54)c3)n2)cc1]

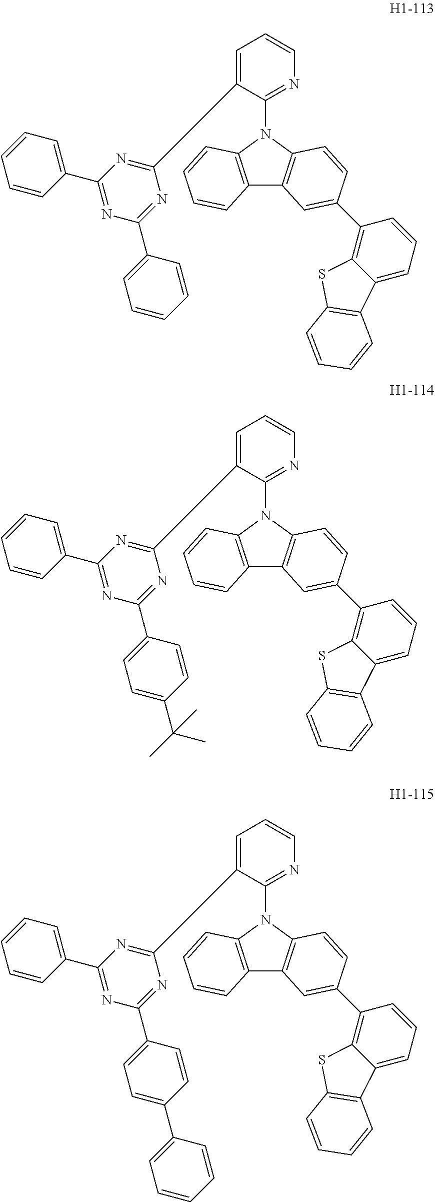 [SMILES: c1ccc(-c2nc(-c3ccccc3)nc(-c3cccnc3-n3c4ccccc4c4cc(-c5cccc6c5sc5ccccc56)ccc43)n2)cc1 ; CC(C)(C)c1ccc(-c2nc(-c3ccccc3)nc(-c3cccnc3-n3c4ccccc4c4cc(-c5cccc6c5sc5ccccc56)ccc43)n2)cc1 ; c1ccc(-c2ccc(-c3nc(-c4ccccc4)nc(-c4cccnc4-n4c5ccccc5c5cc(-c6cccc7c6sc6ccccc67)ccc54)n3)cc2)cc1]

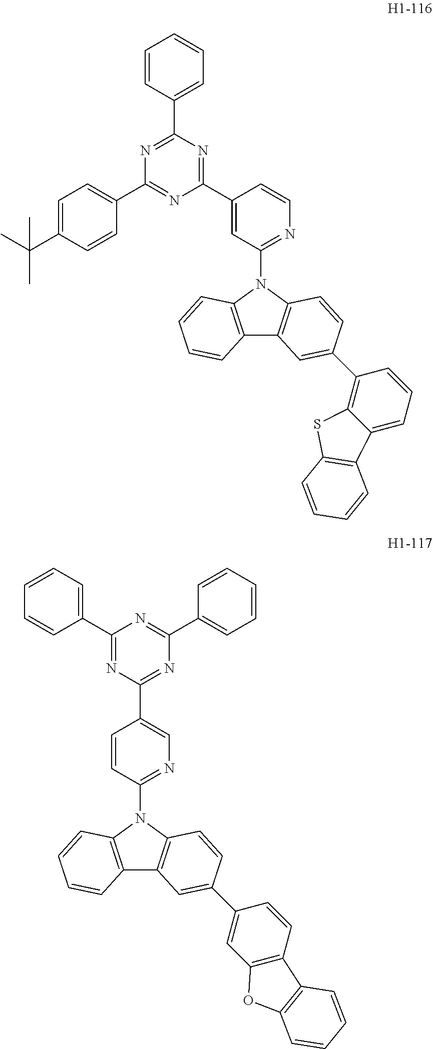 [SMILES: CC(C)(C)c1ccc(-c2nc(-c3ccccc3)nc(-c3ccnc(-n4c5ccccc5c5cc(-c6cccc7c6sc6ccccc67)ccc54)c3)n2)cc1 ; c1ccc(-c2nc(-c3ccccc3)nc(-c3ccc(-n4c5ccccc5c5cc(-c6ccc7c(c6)oc6ccccc67)ccc54)nc3)n2)cc1]

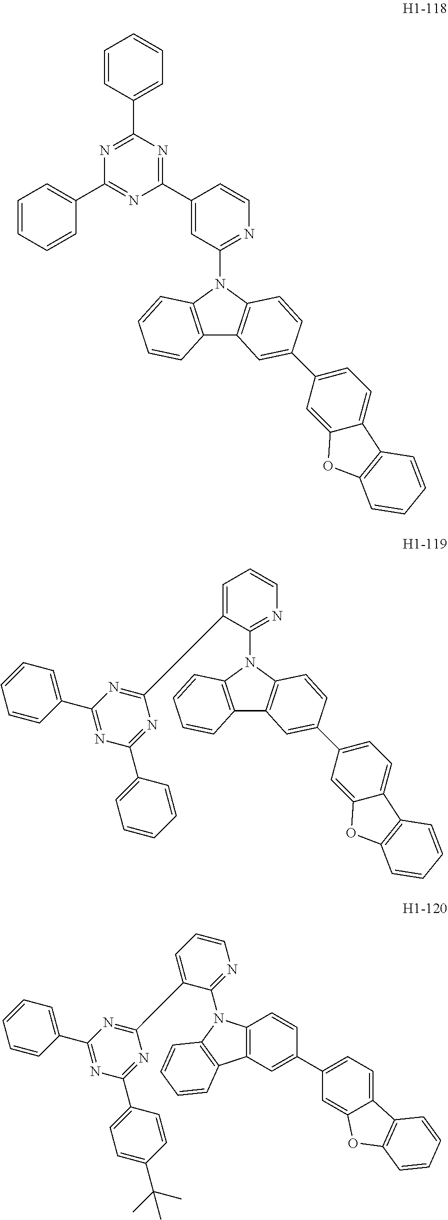 [SMILES: c1ccc(-c2nc(-c3ccccc3)nc(-c3ccnc(-n4c5ccccc5c5cc(-c6ccc7c(c6)oc6ccccc67)ccc54)c3)n2)cc1 ; c1ccc(-c2nc(-c3ccccc3)nc(-c3cccnc3-n3c4ccccc4c4cc(-c5ccc6c(c5)oc5ccccc56)ccc43)n2)cc1 ; CC(C)(C)c1ccc(-c2nc(-c3ccccc3)nc(-c3cccnc3-n3c4ccccc4c4cc(-c5ccc6c(c5)oc5ccccc56)ccc43)n2)cc1]

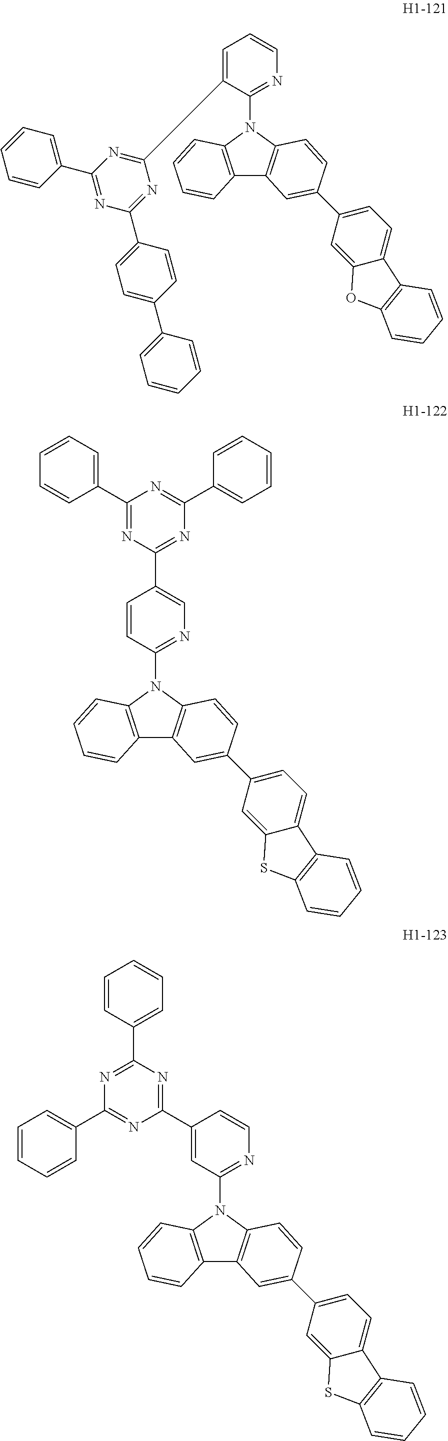 [SMILES: c1ccc(-c2ccc(-c3nc(-c4ccccc4)nc(-c4cccnc4-n4c5ccccc5c5cc(-c6ccc7c(c6)oc6ccccc67)ccc54)n3)cc2)cc1 ; c1ccc(-c2nc(-c3ccccc3)nc(-c3ccc(-n4c5ccccc5c5cc(-c6ccc7c(c6)sc6ccccc67)ccc54)nc3)n2)cc1 ; c1ccc(-c2nc(-c3ccccc3)nc(-c3ccnc(-n4c5ccccc5c5cc(-c6ccc7c(c6)sc6ccccc67)ccc54)c3)n2)cc1]

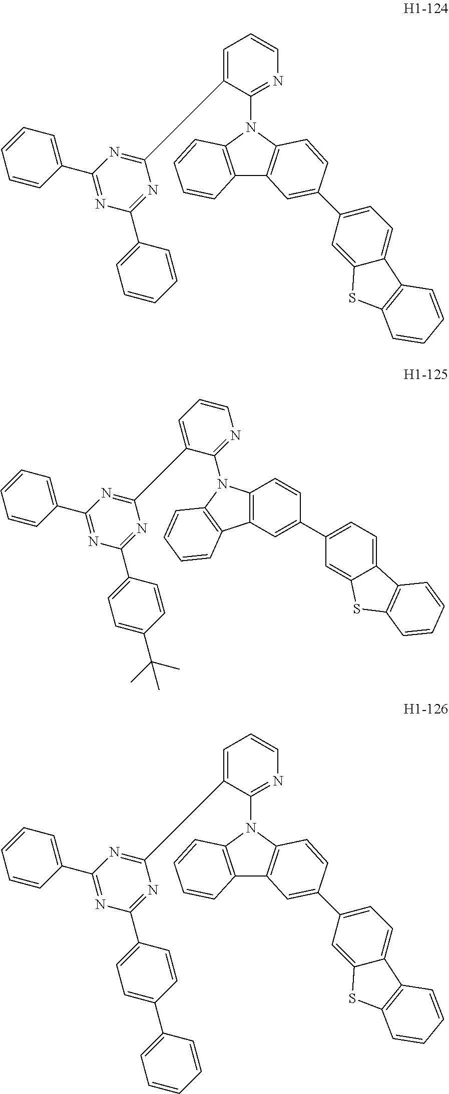 [SMILES: c1ccc(-c2nc(-c3ccccc3)nc(-c3cccnc3-n3c4ccccc4c4cc(-c5ccc6c(c5)sc5ccccc56)ccc43)n2)cc1 ; CC(C)(C)c1ccc(-c2nc(-c3ccccc3)nc(-c3cccnc3-n3c4ccccc4c4cc(-c5ccc6c(c5)sc5ccccc56)ccc43)n2)cc1 ; c1ccc(-c2ccc(-c3nc(-c4ccccc4)nc(-c4cccnc4-n4c5ccccc5c5cc(-c6ccc7c(c6)sc6ccccc67)ccc54)n3)cc2)cc1]

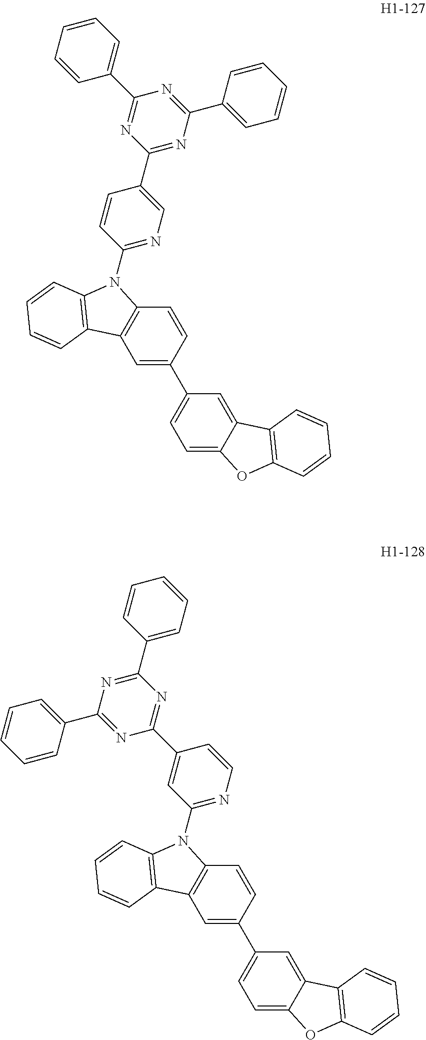 [SMILES: c1ccc(-c2nc(-c3ccccc3)nc(-c3ccc(-n4c5ccccc5c5cc(-c6ccc7oc8ccccc8c7c6)ccc54)nc3)n2)cc1 ; c1ccc(-c2nc(-c3ccccc3)nc(-c3ccnc(-n4c5ccccc5c5cc(-c6ccc7oc8ccccc8c7c6)ccc54)c3)n2)cc1]

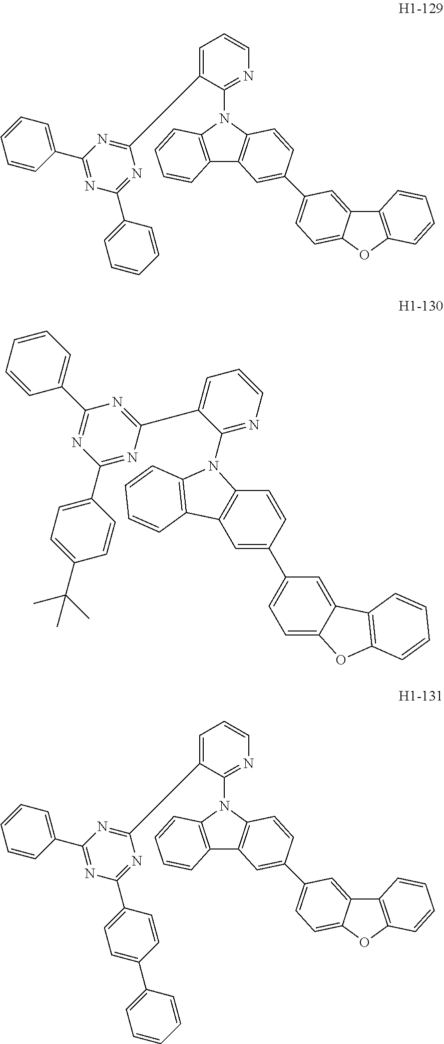 [SMILES: c1ccc(-c2nc(-c3ccccc3)nc(-c3cccnc3-n3c4ccccc4c4cc(-c5ccc6oc7ccccc7c6c5)ccc43)n2)cc1 ; CC(C)(C)c1ccc(-c2nc(-c3ccccc3)nc(-c3cccnc3-n3c4ccccc4c4cc(-c5ccc6oc7ccccc7c6c5)ccc43)n2)cc1 ; c1ccc(-c2ccc(-c3nc(-c4ccccc4)nc(-c4cccnc4-n4c5ccccc5c5cc(-c6ccc7oc8ccccc8c7c6)ccc54)n3)cc2)cc1]

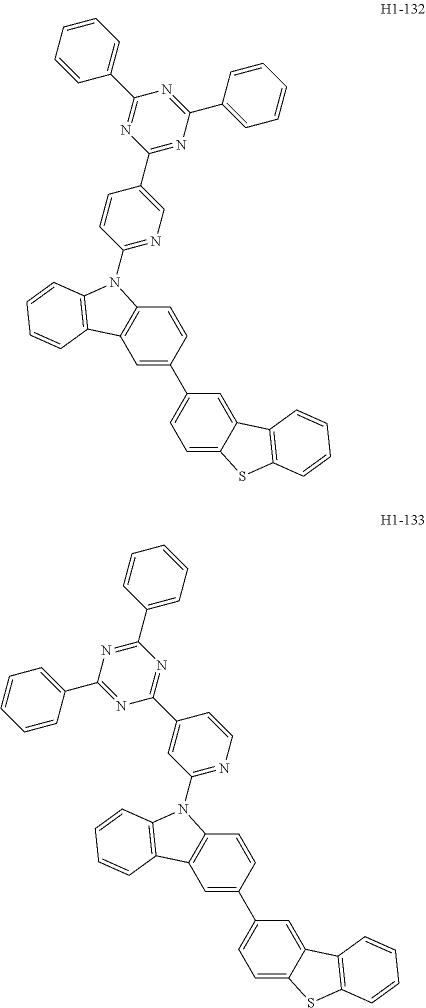 [SMILES: c1ccc(-c2nc(-c3ccccc3)nc(-c3ccc(-n4c5ccccc5c5cc(-c6ccc7sc8ccccc8c7c6)ccc54)nc3)n2)cc1 ; c1ccc(-c2nc(-c3ccccc3)nc(-c3ccnc(-n4c5ccccc5c5cc(-c6ccc7sc8ccccc8c7c6)ccc54)c3)n2)cc1]

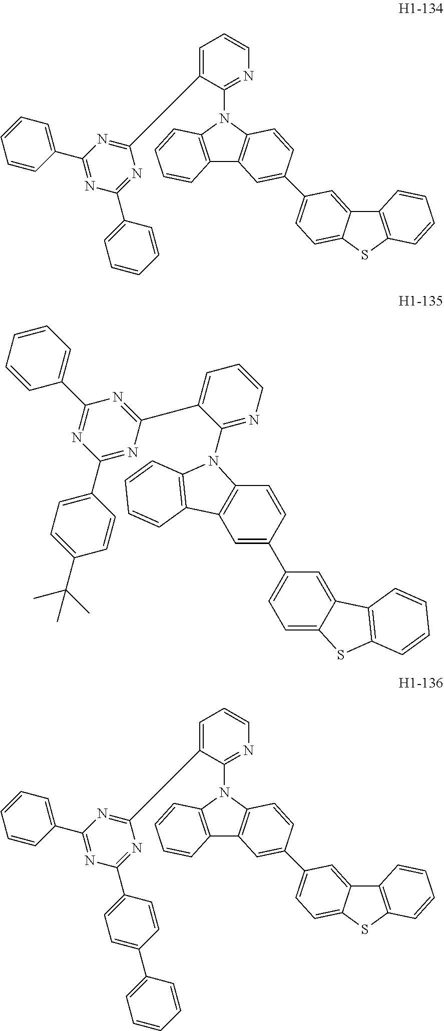 [SMILES: c1ccc(-c2nc(-c3ccccc3)nc(-c3cccnc3-n3c4ccccc4c4cc(-c5ccc6sc7ccccc7c6c5)ccc43)n2)cc1 ; CC(C)(C)c1ccc(-c2nc(-c3ccccc3)nc(-c3cccnc3-n3c4ccccc4c4cc(-c5ccc6sc7ccccc7c6c5)ccc43)n2)cc1 ; c1ccc(-c2ccc(-c3nc(-c4ccccc4)nc(-c4cccnc4-n4c5ccccc5c5cc(-c6ccc7sc8ccccc8c7c6)ccc54)n3)cc2)cc1]

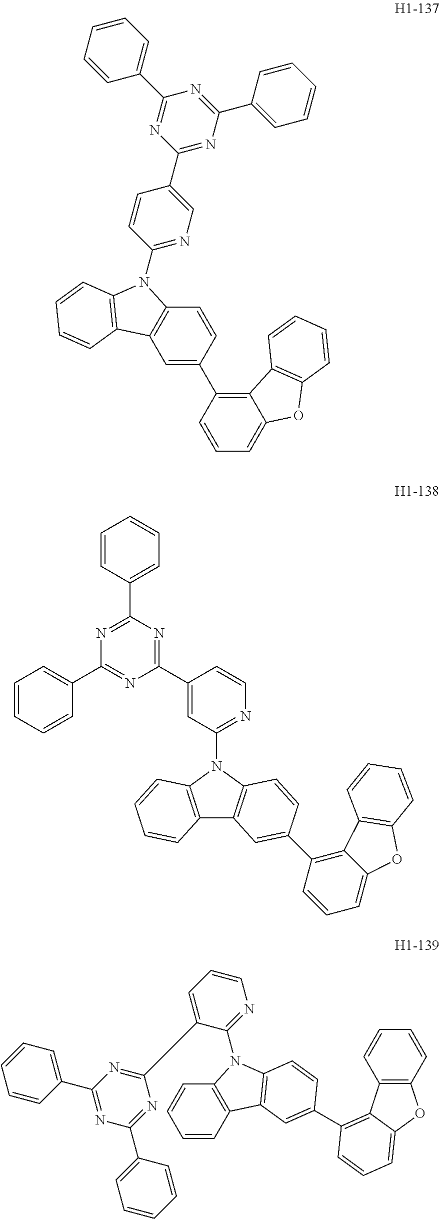 [SMILES: c1ccc(-c2nc(-c3ccccc3)nc(-c3ccc(-n4c5ccccc5c5cc(-c6cccc7oc8ccccc8c67)ccc54)nc3)n2)cc1 ; c1ccc(-c2nc(-c3ccccc3)nc(-c3ccnc(-n4c5ccccc5c5cc(-c6cccc7oc8ccccc8c67)ccc54)c3)n2)cc1 ; c1ccc(-c2nc(-c3ccccc3)nc(-c3cccnc3-n3c4ccccc4c4cc(-c5cccc6oc7ccccc7c56)ccc43)n2)cc1]

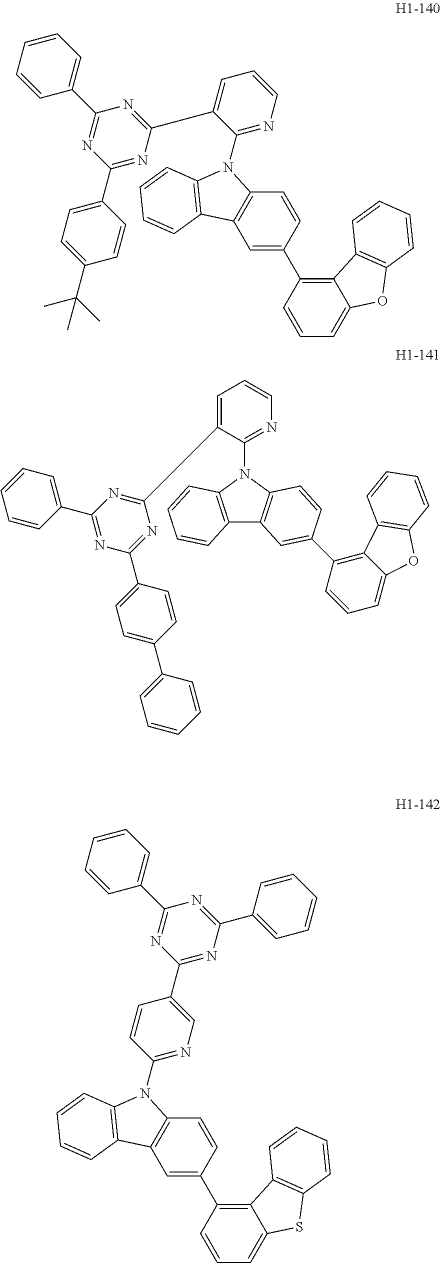 [SMILES: CC(C)(C)c1ccc(-c2nc(-c3ccccc3)nc(-c3cccnc3-n3c4ccccc4c4cc(-c5cccc6oc7ccccc7c56)ccc43)n2)cc1 ; c1ccc(-c2ccc(-c3nc(-c4ccccc4)nc(-c4cccnc4-n4c5ccccc5c5cc(-c6cccc7oc8ccccc8c67)ccc54)n3)cc2)cc1 ; c1ccc(-c2nc(-c3ccccc3)nc(-c3ccc(-n4c5ccccc5c5cc(-c6cccc7sc8ccccc8c67)ccc54)nc3)n2)cc1]

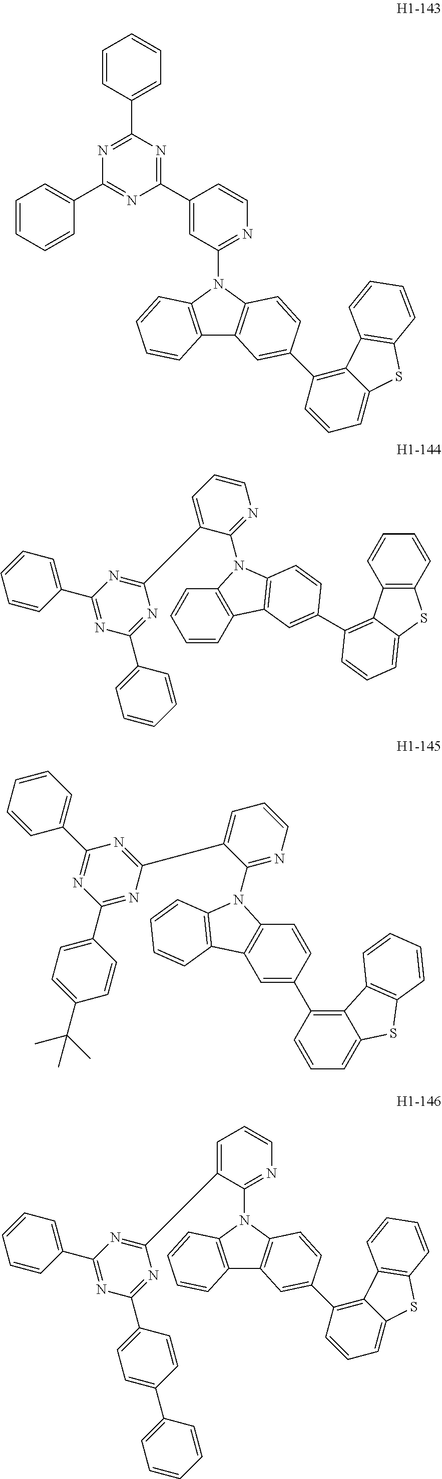 [SMILES: c1ccc(-c2nc(-c3ccccc3)nc(-c3ccnc(-n4c5ccccc5c5cc(-c6cccc7sc8ccccc8c67)ccc54)c3)n2)cc1 ; c1ccc(-c2nc(-c3ccccc3)nc(-c3cccnc3-n3c4ccccc4c4cc(-c5cccc6sc7ccccc7c56)ccc43)n2)cc1 ; CC(C)(C)c1ccc(-c2nc(-c3ccccc3)nc(-c3cccnc3-n3c4ccccc4c4cc(-c5cccc6sc7ccccc7c56)ccc43)n2)cc1 ; c1ccc(-c2ccc(-c3nc(-c4ccccc4)nc(-c4cccnc4-n4c5ccccc5c5cc(-c6cccc7sc8ccccc8c67)ccc54)n3)cc2)cc1]

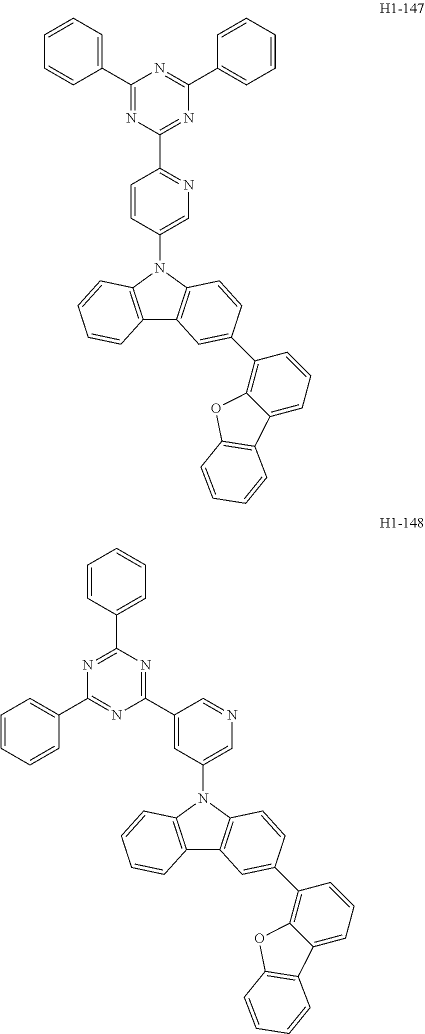 [SMILES: c1ccc(-c2nc(-c3ccccc3)nc(-c3ccc(-n4c5ccccc5c5cc(-c6cccc7c6oc6ccccc67)ccc54)cn3)n2)cc1 ; c1ccc(-c2nc(-c3ccccc3)nc(-c3cncc(-n4c5ccccc5c5cc(-c6cccc7c6oc6ccccc67)ccc54)c3)n2)cc1]

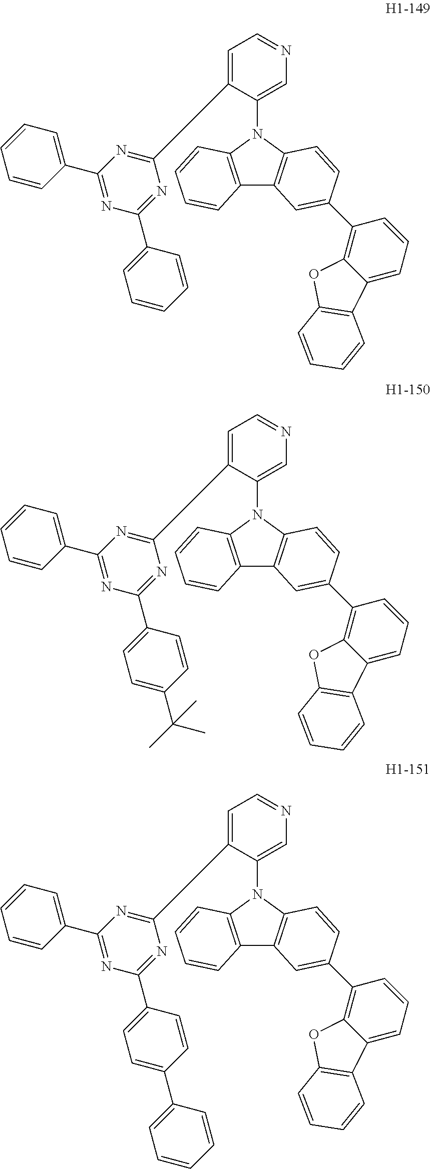 [SMILES: c1ccc(-c2nc(-c3ccccc3)nc(-c3ccncc3-n3c4ccccc4c4cc(-c5cccc6c5oc5ccccc56)ccc43)n2)cc1 ; CC(C)(C)c1ccc(-c2nc(-c3ccccc3)nc(-c3ccncc3-n3c4ccccc4c4cc(-c5cccc6c5oc5ccccc56)ccc43)n2)cc1 ; c1ccc(-c2ccc(-c3nc(-c4ccccc4)nc(-c4ccncc4-n4c5ccccc5c5cc(-c6cccc7c6oc6ccccc67)ccc54)n3)cc2)cc1]

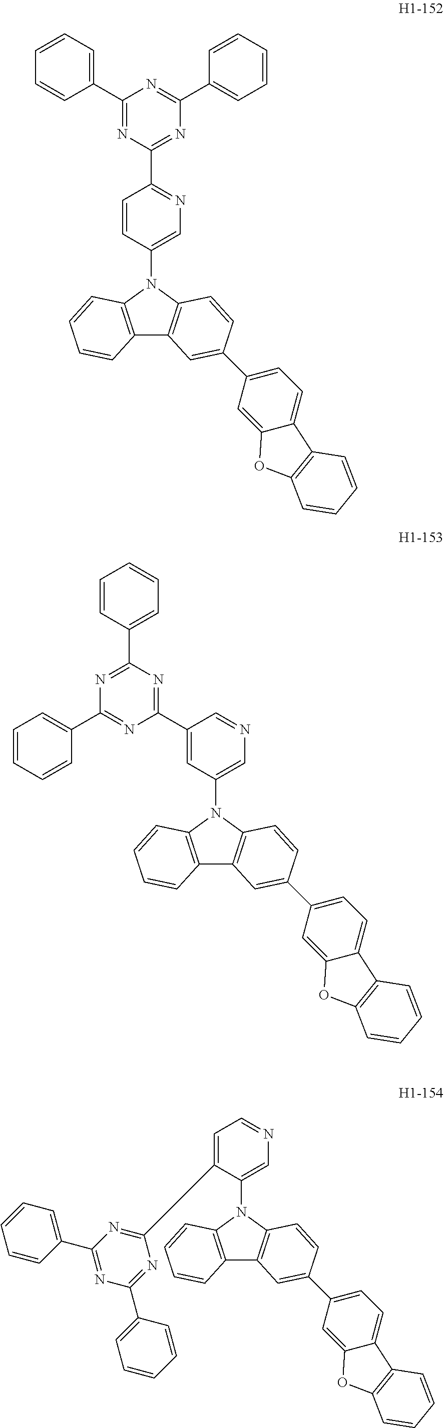 [SMILES: c1ccc(-c2nc(-c3ccccc3)nc(-c3ccc(-n4c5ccccc5c5cc(-c6ccc7c(c6)oc6ccccc67)ccc54)cn3)n2)cc1 ; c1ccc(-c2nc(-c3ccccc3)nc(-c3cncc(-n4c5ccccc5c5cc(-c6ccc7c(c6)oc6ccccc67)ccc54)c3)n2)cc1 ; c1ccc(-c2nc(-c3ccccc3)nc(-c3ccncc3-n3c4ccccc4c4cc(-c5ccc6c(c5)oc5ccccc56)ccc43)n2)cc1]

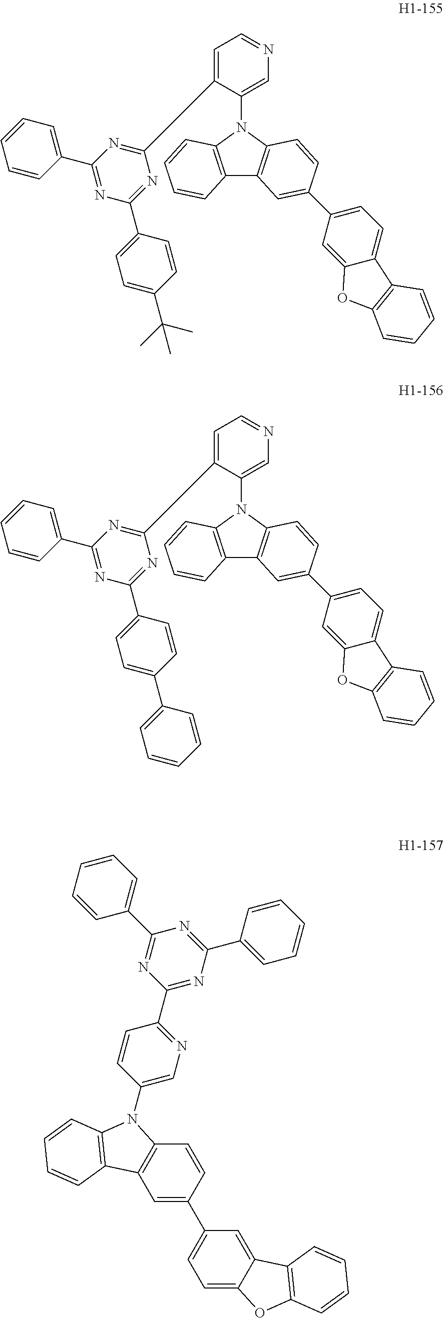 [SMILES: CC(C)(C)c1ccc(-c2nc(-c3ccccc3)nc(-c3ccncc3-n3c4ccccc4c4cc(-c5ccc6c(c5)oc5ccccc56)ccc43)n2)cc1 ; c1ccc(-c2ccc(-c3nc(-c4ccccc4)nc(-c4ccncc4-n4c5ccccc5c5cc(-c6ccc7c(c6)oc6ccccc67)ccc54)n3)cc2)cc1 ; c1ccc(-c2nc(-c3ccccc3)nc(-c3ccc(-n4c5ccccc5c5cc(-c6ccc7oc8ccccc8c7c6)ccc54)cn3)n2)cc1]

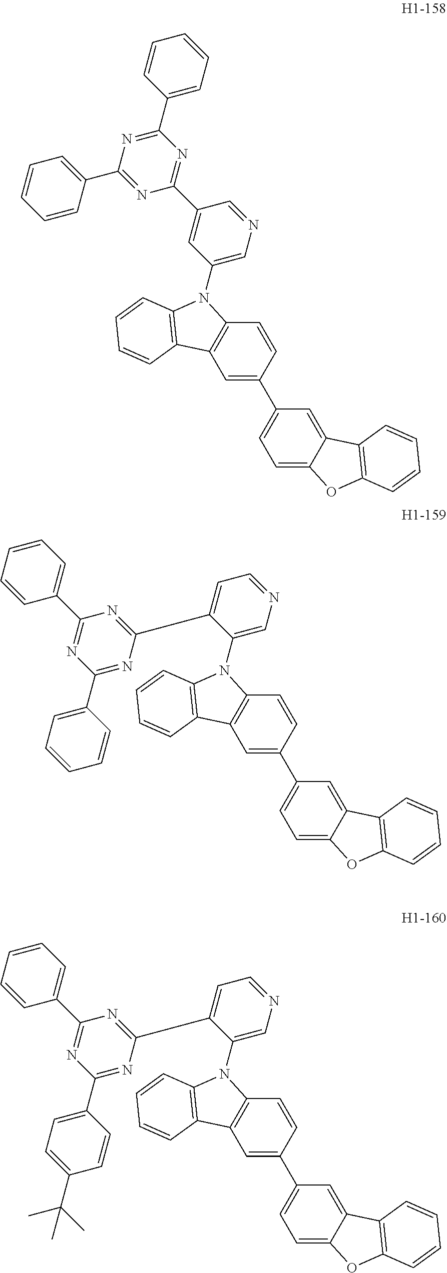 [SMILES: c1ccc(-c2nc(-c3ccccc3)nc(-c3cncc(-n4c5ccccc5c5cc(-c6ccc7oc8ccccc8c7c6)ccc54)c3)n2)cc1 ; c1ccc(-c2nc(-c3ccccc3)nc(-c3ccncc3-n3c4ccccc4c4cc(-c5ccc6oc7ccccc7c6c5)ccc43)n2)cc1 ; CC(C)(C)c1ccc(-c2nc(-c3ccccc3)nc(-c3ccncc3-n3c4ccccc4c4cc(-c5ccc6oc7ccccc7c6c5)ccc43)n2)cc1]

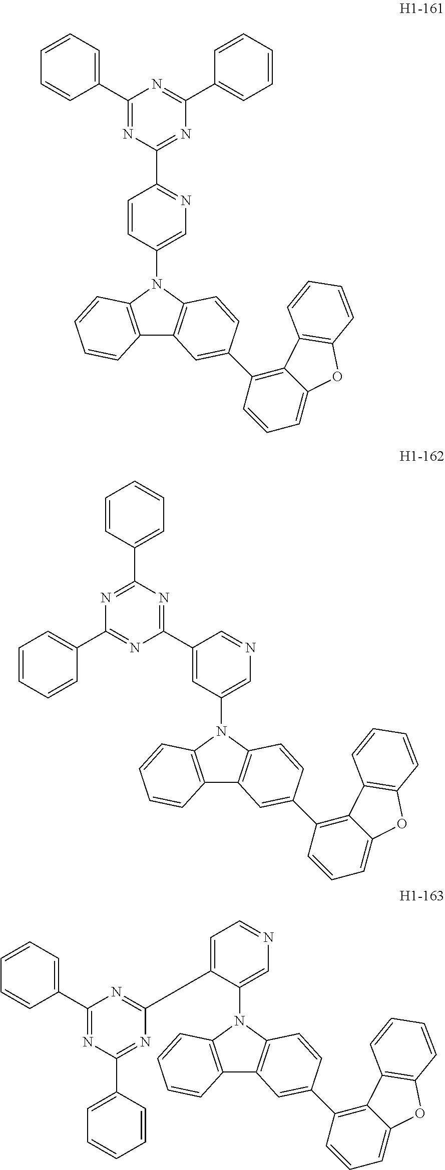 [SMILES: c1ccc(-c2nc(-c3ccccc3)nc(-c3ccc(-n4c5ccccc5c5cc(-c6cccc7oc8ccccc8c67)ccc54)cn3)n2)cc1 ; c1ccc(-c2nc(-c3ccccc3)nc(-c3cncc(-n4c5ccccc5c5cc(-c6cccc7oc8ccccc8c67)ccc54)c3)n2)cc1 ; c1ccc(-c2nc(-c3ccccc3)nc(-c3ccncc3-n3c4ccccc4c4cc(-c5cccc6oc7ccccc7c56)ccc43)n2)cc1]

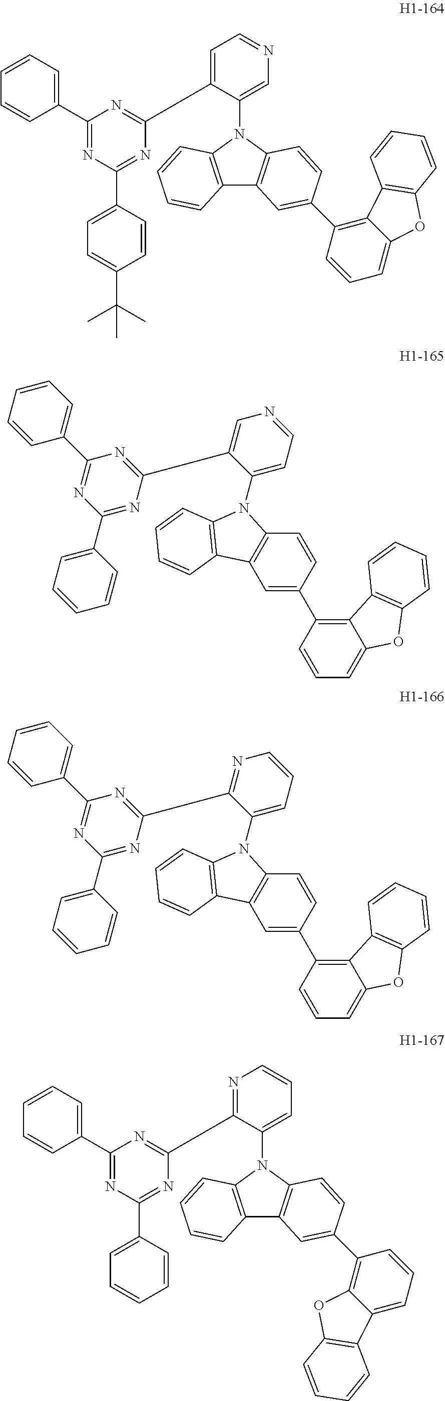 [SMILES: CC(C)(C)c1ccc(-c2nc(-c3ccccc3)nc(-c3ccncc3-n3c4ccccc4c4cc(-c5cccc6oc7ccccc7c56)ccc43)n2)cc1 ; c1ccc(-c2nc(-c3ccccc3)nc(-c3cnccc3-n3c4ccccc4c4cc(-c5cccc6oc7ccccc7c56)ccc43)n2)cc1 ; c1ccc(-c2nc(-c3ccccc3)nc(-c3ncccc3-n3c4ccccc4c4cc(-c5cccc6oc7ccccc7c56)ccc43)n2)cc1 ; c1ccc(-c2nc(-c3ccccc3)nc(-c3ncccc3-n3c4ccccc4c4cc(-c5cccc6c5oc5ccccc56)ccc43)n2)cc1]

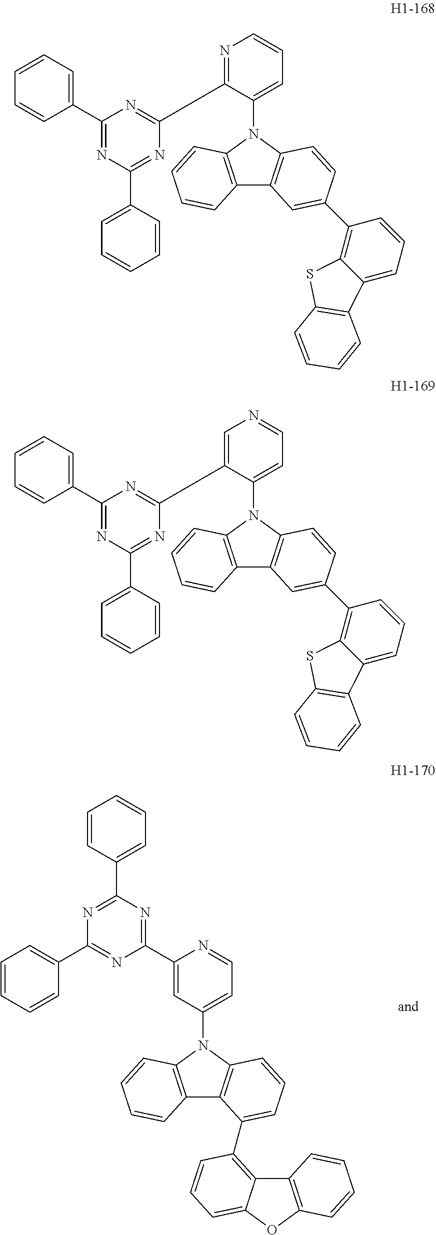 [SMILES: c1ccc(-c2nc(-c3ccccc3)nc(-c3ncccc3-n3c4ccccc4c4cc(-c5cccc6c5sc5ccccc56)ccc43)n2)cc1 ; c1ccc(-c2nc(-c3ccccc3)nc(-c3cnccc3-n3c4ccccc4c4cc(-c5cccc6c5sc5ccccc56)ccc43)n2)cc1 ; c1ccc(-c2nc(-c3ccccc3)nc(-c3cc(-n4c5ccccc5c5c(-c6cccc7oc8ccccc8c67)cccc54)ccn3)n2)cc1]

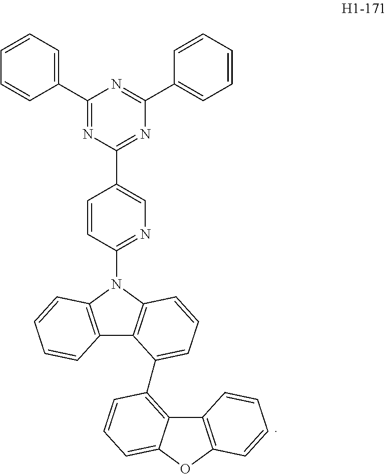 [SMILES: c1ccc(-c2nc(-c3ccccc3)nc(-c3ccc(-n4c5ccccc5c5c(-c6cccc7oc8ccccc8c67)cccc54)nc3)n2)cc1]

6. A host material comprising the organic electroluminescent compound represented by formula 1 according to claim 1.

7. A plurality of host materials comprising the host material according to claim 6 as a first host material and further comprising a second host material comprising a compound represented by the following formula 11: 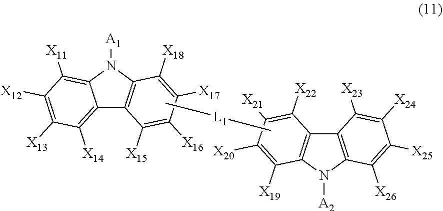
wherein
A₁ and A₂, each independently, represent a substituted or unsubstituted (C6-C30)aryl, or a substituted or unsubstituted (3- to 30-membered)heteroaryl;
L₁ represents a single bond, or a substituted or unsubstituted (C6-C30)arylene; and
X₁₁ to X₂₆, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted (3- to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono-or di- (C1-C30)alkylamino, or a substituted or unsubstituted mono- or di- (C6-C30)arylamino; or at least two adjacent ones of X₁₁ to X₂₆ may be linked to each other to form a ring(s).

8. The plurality of host materials according to claim 7, wherein the compound represented by formula 11 is selected from the following compounds:

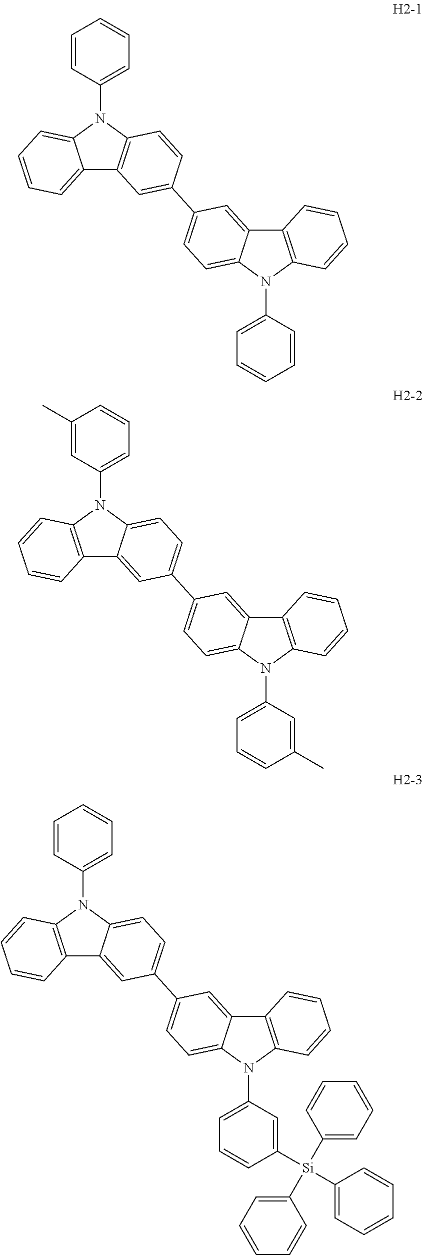 [SMILES: c1ccc(-n2c3ccccc3c3cc(-c4ccc5c(c4)c4ccccc4n5-c4ccccc4)ccc32)cc1 ; Cc1cccc(-n2c3ccccc3c3cc(-c4ccc5c(c4)c4ccccc4n5-c4cccc(C)c4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

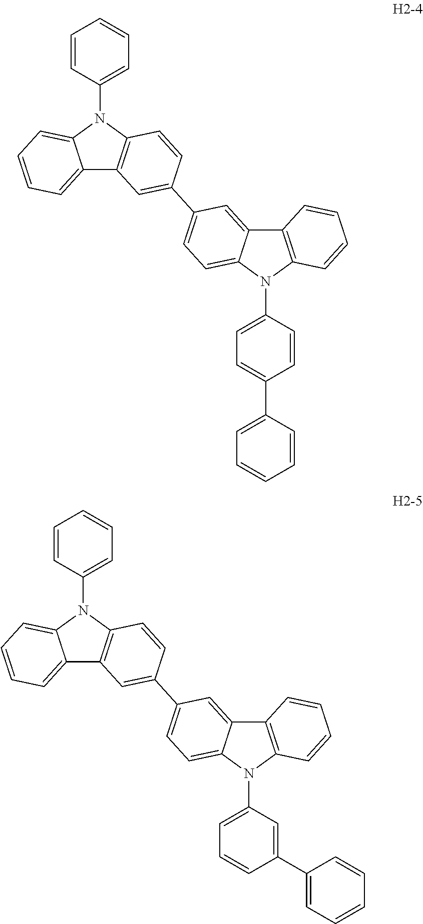 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1]

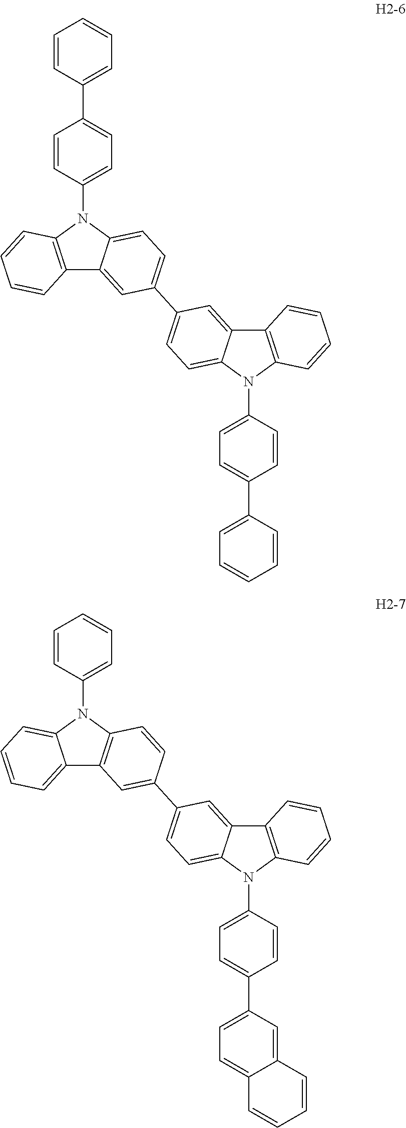 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-n2c3ccccc3c3cc(-c4ccc5c(c4)c4ccccc4n5-c4ccc(-c5ccc6ccccc6c5)cc4)ccc32)cc1]

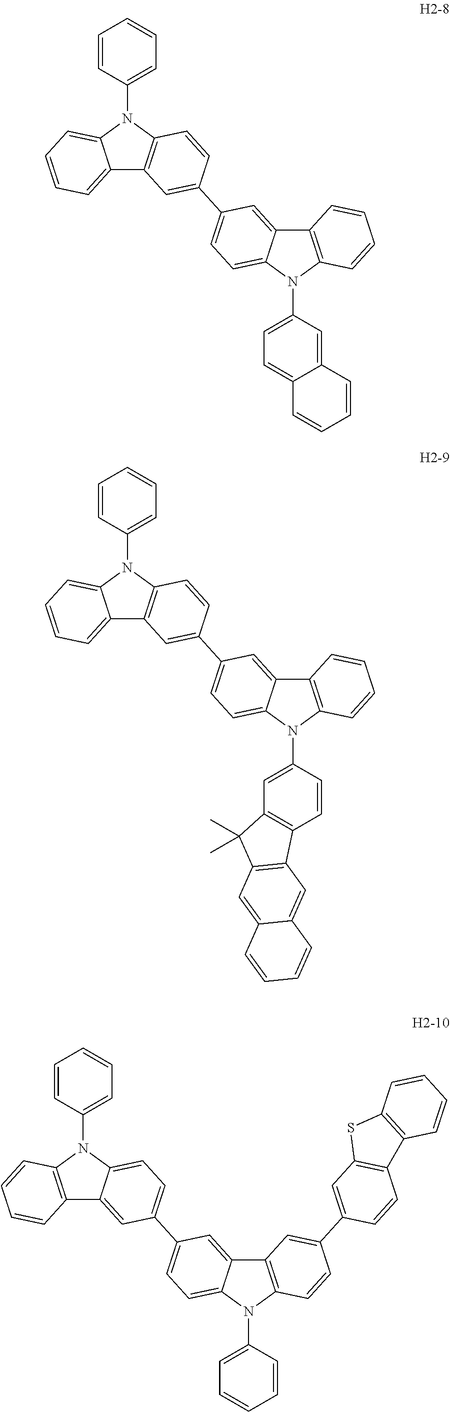 [SMILES: c1ccc(-n2c3ccccc3c3cc(-c4ccc5c(c4)c4ccccc4n5-c4ccc5ccccc5c4)ccc32)cc1 ; CC1(C)c2cc(-n3c4ccccc4c4cc(-c5ccc6c(c5)c5ccccc5n6-c5ccccc5)ccc43)ccc2-c2cc3ccccc3cc21 ; c1ccc(-n2c3ccccc3c3cc(-c4ccc5c(c4)c4cc(-c6ccc7c(c6)sc6ccccc67)ccc4n5-c4ccccc4)ccc32)cc1]

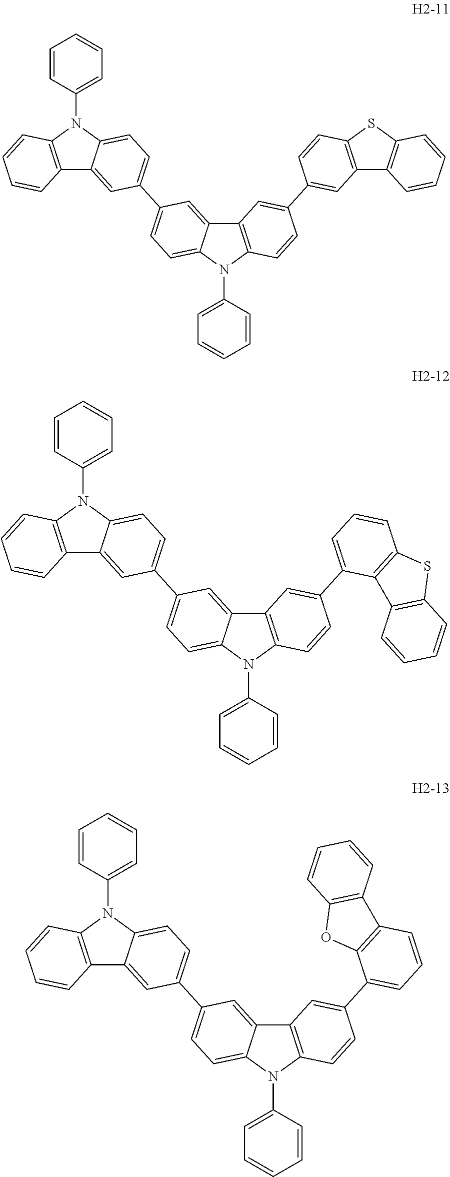 [SMILES: c1ccc(-n2c3ccccc3c3cc(-c4ccc5c(c4)c4cc(-c6ccc7sc8ccccc8c7c6)ccc4n5-c4ccccc4)ccc32)cc1 ; c1ccc(-n2c3ccccc3c3cc(-c4ccc5c(c4)c4cc(-c6cccc7sc8ccccc8c67)ccc4n5-c4ccccc4)ccc32)cc1 ; c1ccc(-n2c3ccccc3c3cc(-c4ccc5c(c4)c4cc(-c6cccc7c6oc6ccccc67)ccc4n5-c4ccccc4)ccc32)cc1]

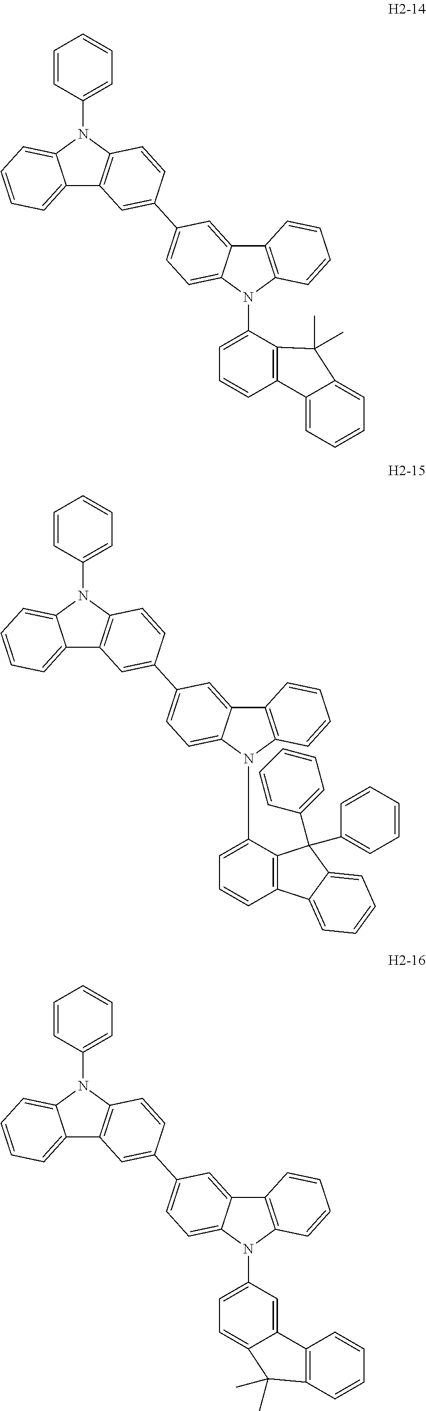 [SMILES: CC1(C)c2ccccc2-c2cccc(-n3c4ccccc4c4cc(-c5ccc6c(c5)c5ccccc5n6-c5ccccc5)ccc43)c21 ; c1ccc(-n2c3ccccc3c3cc(-c4ccc5c(c4)c4ccccc4n5-c4cccc5c4C(c4ccccc4)(c4ccccc4)c4ccccc4-5)ccc32)cc1 ; CC1(C)c2ccccc2-c2cc(-n3c4ccccc4c4cc(-c5ccc6c(c5)c5ccccc5n6-c5ccccc5)ccc43)ccc21]

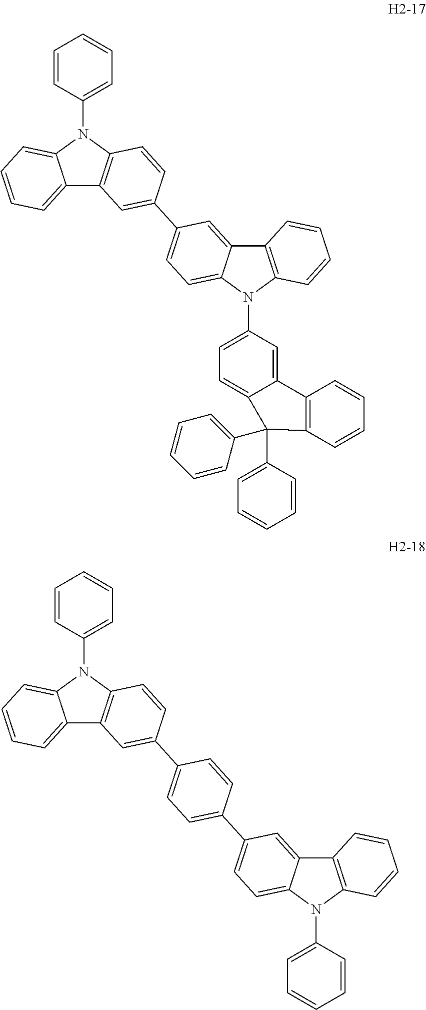 [SMILES: c1ccc(-n2c3ccccc3c3cc(-c4ccc5c(c4)c4ccccc4n5-c4ccc5c(c4)-c4ccccc4C5(c4ccccc4)c4ccccc4)ccc32)cc1 ; c1ccc(-n2c3ccccc3c3cc(-c4ccc(-c5ccc6c(c5)c5ccccc5n6-c5ccccc5)cc4)ccc32)cc1]

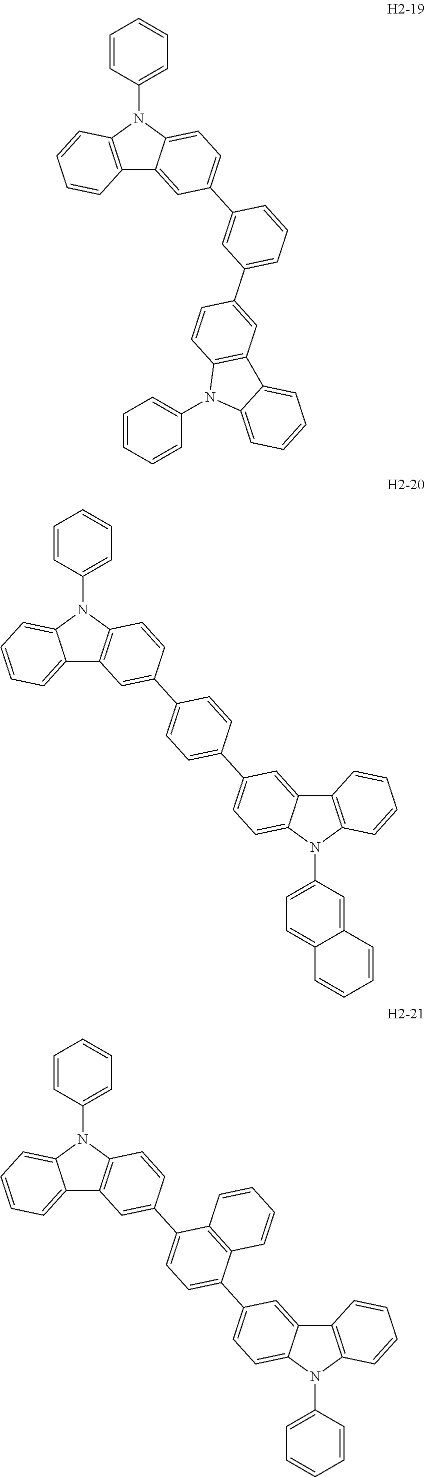 [SMILES: c1ccc(-n2c3ccccc3c3cc(-c4cccc(-c5ccc6c(c5)c5ccccc5n6-c5ccccc5)c4)ccc32)cc1 ; c1ccc(-n2c3ccccc3c3cc(-c4ccc(-c5ccc6c(c5)c5ccccc5n6-c5ccc6ccccc6c5)cc4)ccc32)cc1 ; c1ccc(-n2c3ccccc3c3cc(-c4ccc(-c5ccc6c(c5)c5ccccc5n6-c5ccccc5)c5ccccc45)ccc32)cc1]

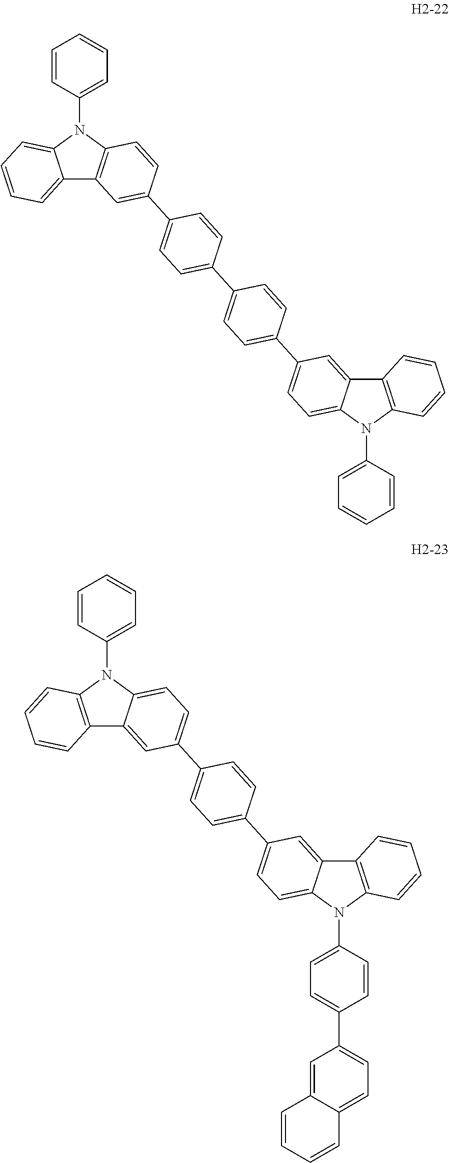 [SMILES: c1ccc(-n2c3ccccc3c3cc(-c4ccc(-c5ccc(-c6ccc7c(c6)c6ccccc6n7-c6ccccc6)cc5)cc4)ccc32)cc1 ; c1ccc(-n2c3ccccc3c3cc(-c4ccc(-c5ccc6c(c5)c5ccccc5n6-c5ccc(-c6ccc7ccccc7c6)cc5)cc4)ccc32)cc1]

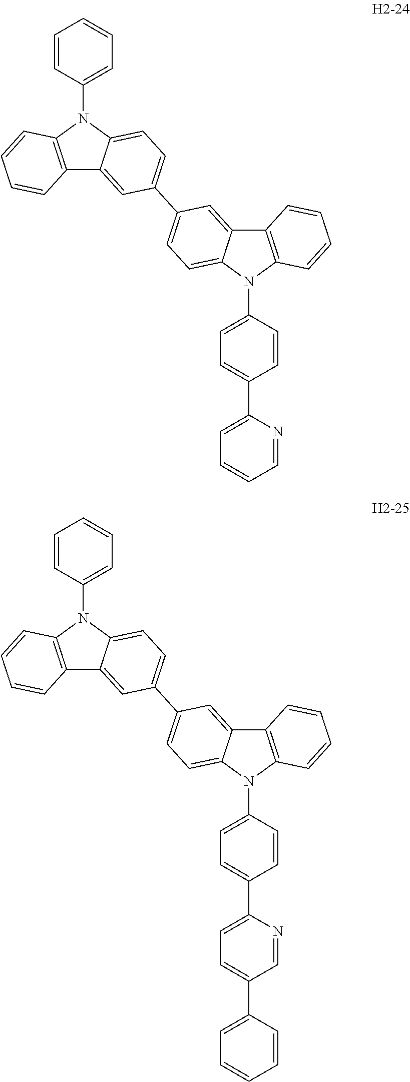 [SMILES: c1ccc(-n2c3ccccc3c3cc(-c4ccc5c(c4)c4ccccc4n5-c4ccc(-c5ccccn5)cc4)ccc32)cc1 ; c1ccc(-c2ccc(-c3ccc(-n4c5ccccc5c5cc(-c6ccc7c(c6)c6ccccc6n7-c6ccccc6)ccc54)cc3)nc2)cc1]

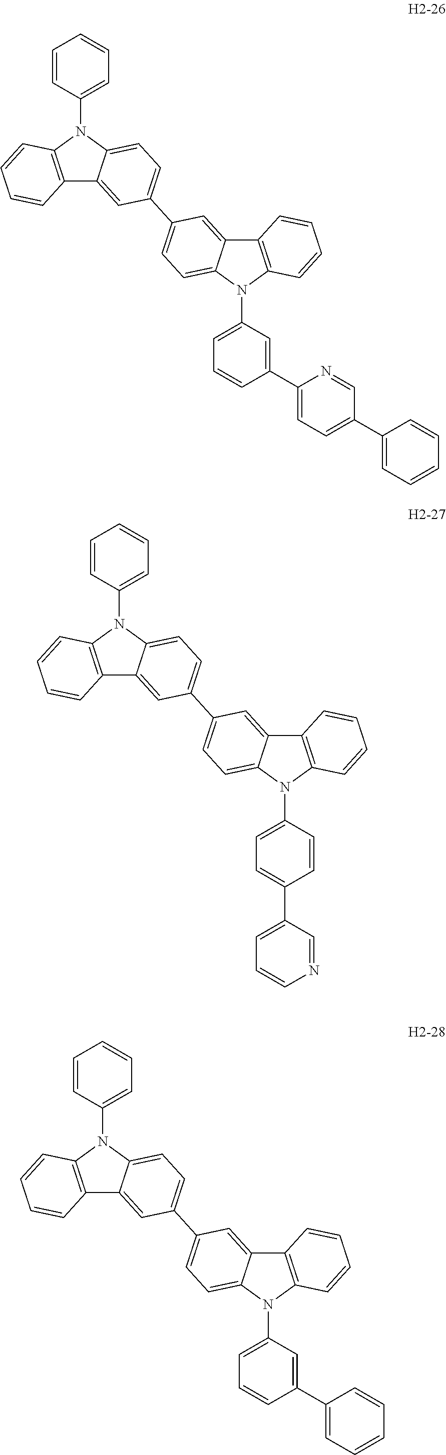 [SMILES: c1ccc(-c2ccc(-c3cccc(-n4c5ccccc5c5cc(-c6ccc7c(c6)c6ccccc6n7-c6ccccc6)ccc54)c3)nc2)cc1 ; c1ccc(-n2c3ccccc3c3cc(-c4ccc5c(c4)c4ccccc4n5-c4ccc(-c5cccnc5)cc4)ccc32)cc1 ; c1ccc(-c2cccc(-n3c4ccccc4c4cc(-c5ccc6c(c5)c5ccccc5n6-c5ccccc5)ccc43)c2)cc1]

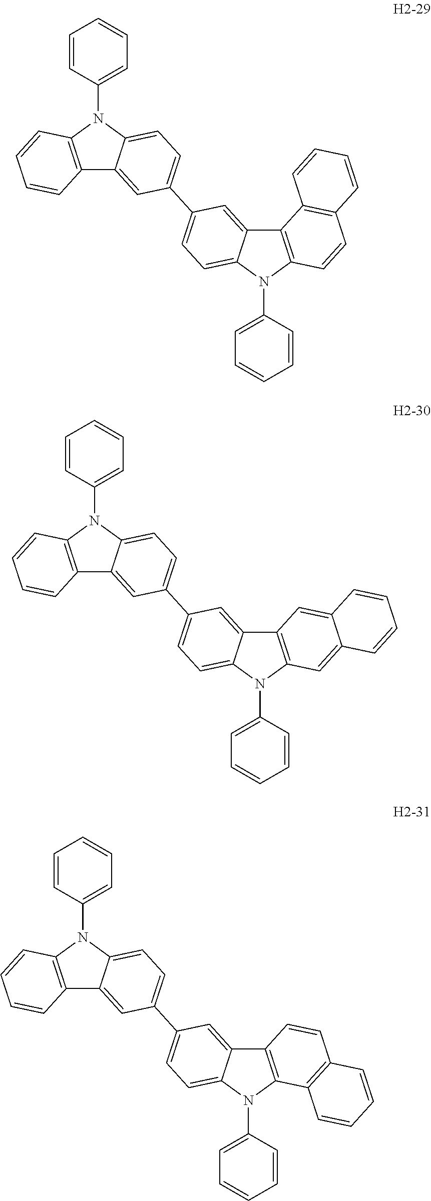 [SMILES: c1ccc(-n2c3ccccc3c3cc(-c4ccc5c(c4)c4c6ccccc6ccc4n5-c4ccccc4)ccc32)cc1 ; c1ccc(-n2c3ccccc3c3cc(-c4ccc5c(c4)c4cc6ccccc6cc4n5-c4ccccc4)ccc32)cc1 ; c1ccc(-n2c3ccccc3c3cc(-c4ccc5c(c4)c4ccc6ccccc6c4n5-c4ccccc4)ccc32)cc1]

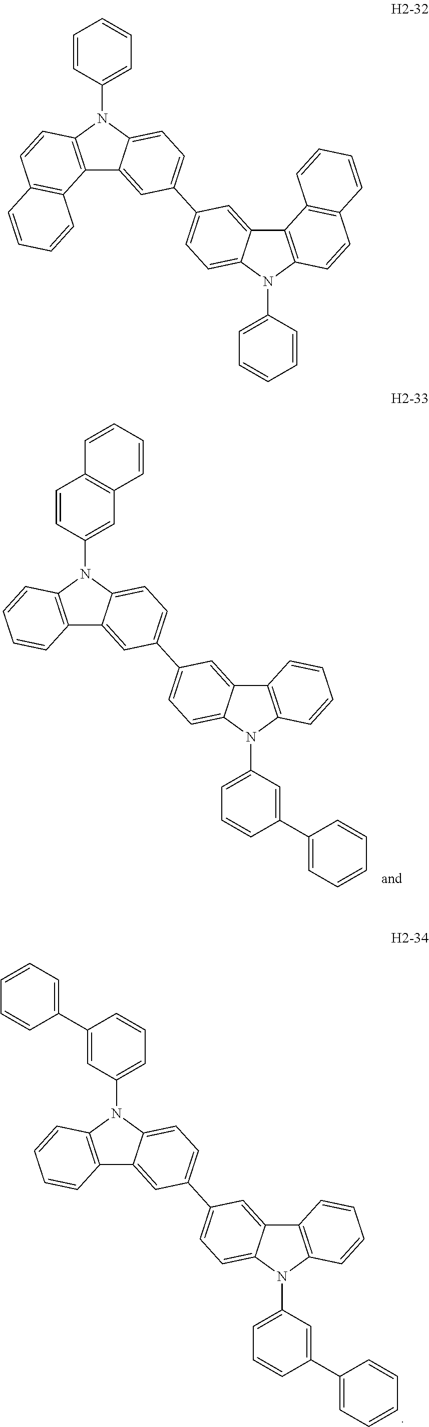 [SMILES: c1ccc(-n2c3ccc(-c4ccc5c(c4)c4c6ccccc6ccc4n5-c4ccccc4)cc3c3c4ccccc4ccc32)cc1 ; c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5cccc(-c6ccccc6)c5)ccc43)c2)cc1]

9. An organic electroluminescent device comprising the organic electroluminescent compound according to claim 1.

10. An organic electroluminescent device comprising the plurality of host materials according to claim 7.
